SEQUENCE LISTING (55 sequences)

SEQ ID NO: 1 - DNA, 1035 bp, Artificial Sequence
  Synthetic chimeric nucleotide sequence
atggccctgttggtgcacttcctacccctgctggccctgcttgccctctgggagcccaaacccacccaggcttttgtcaaacagcatctttgtggtccccacctggtagaggctctctacctggtgtgtggggagcgtggcttcttctacacacccaagtcccgccgtgaagtggaggacccacaagtggaacaactggagctgggaggaagccccggggaccttcagaccttggcgttggaggtggcccggcagaagcgtggcattgtggatcagtgctgcaccagcatctgctccctctaccagctggagaactactgcaacggtggcggcggtagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaatgagtgctagct

SEQ ID NO: 2 - protein, 341 aa, Artificial Sequence
  Synthetic chimeric protein
MALLVHFLPLLALLALWEPKPTQAFVKQHLCGPHLVEALYLVCGERGFFYTPKSRREVEDPQVEQLELGGSPGDLQTLALEVARQKRGIVDQCCTSICSLYQLENYCNGGGGRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, Artificial Sequence
  Synthetic epitope
SFERFEIFPK

SEQ ID NO: 4 - DNA, 30 bp, Artificial Sequence
  Synthetic primer
ctagctagcaccatgtacaggatgcaactc

SEQ ID NO: 5 - DNA, 54 bp, Artificial Sequence
  Synthetic primer
attgcactaagtcttgcacttgctacgaactcggagctcgagcagaaactcatc

SEQ ID NO: 6 - DNA, 52 bp, Artificial Sequence
  Synthetic primer
gcgagctcgagcagaaactcatctctgaagaggatctgggatccagatcttc

SEQ ID NO: 7 - DNA, 52 bp, Artificial Sequence
  Synthetic primer
gcgagctcgagcagaaactcatctctgaagaggatctggaattcagatcttc

SEQ ID NO: 8 - DNA, 27 bp, Artificial Sequence
  Synthetic primer
cgcggatccgacacaatatgtataggc

SEQ ID NO: 9 - DNA, 27 bp, Artificial Sequence
  Synthetic primer
ggaagatctggattgaatggacggagt

SEQ ID NO: 10 - DNA, 24 bp, Artificial Sequence
  Synthetic primer
ggattctcagttgccaagaagcat

SEQ ID NO: 11 - DNA, 24 bp, Artificial Sequence
  Synthetic primer
gcgagatcttggttcaatggcatt

SEQ ID NO: 12 - DNA, 26 bp, Artificial Sequence
  Synthetic primer
cgggatccaatagttgcagcatgcca

SEQ ID NO: 13 - DNA, 26 bp, Artificial Sequence
  Synthetic primer
gccagatctgtagaggtcctgtgcct

SEQ ID NO: 14 - DNA, 29 bp, Artificial Sequence
  Synthetic primer
gcgagatctggttgtaagccttgcatatg

SEQ ID NO: 15 - DNA, 28 bp, Artificial Sequence
  Synthetic primer
cggatatcggatcatttaccaggagagt

SEQ ID NO: 16 - DNA, 20 bp, Artificial Sequence
  Synthetic primer
taatacgactcactataggg

SEQ ID NO: 17 - DNA, 18 bp, Artificial Sequence
  Artificial pirmer
tagaaggcacagtcgagg

SEQ ID NO: 18 - DNA, 21 bp, Artificial Sequence
  Synthetic primer
atggccctgttggtgcacttc

SEQ ID NO: 19 - DNA, 36 bp, Artificial Sequence
  Synthetic primer
agatctaccgccgccaccgttgcagtagttctccag

SEQ ID NO: 20 - DNA, 30 bp, Artificial Sequence
  Synthetic primer
atgatatcaggccctgttggtgcacttcct

SEQ ID NO: 21 - DNA, 36 bp, Artificial Sequence
  Synthetic primer
tagatctaccgccgccaccgttgcagtagttctcca

SEQ ID NO: 22 - DNA, 20 bp, Artificial Sequence
  Synthetic primer
aatttctagaatggccctgt

SEQ ID NO: 23 - DNA, 23 bp, Artificial Sequence
  Synthetic primer
aattctcgagctagttgcagtag

SEQ ID NO: 24 - DNA, 21 bp, Artificial Sequence
  Synthetic primer
atggccctgtggatgcgcttc

SEQ ID NO: 25 - DNA, 36 bp, Artificial Sequence; identical to SEQ ID NO: 19
  Synthetic primer

SEQ ID NO: 26 - DNA, 30 bp, Artificial Sequence
  Synthetic primer
atgatacatggccctgtggatgcgcttcct

SEQ ID NO: 27 - DNA, 36 bp, Artificial Sequence; identical to SEQ ID NO: 21
  Synthetic primer

SEQ ID NO: 28 - DNA, 20 bp, Artificial Sequence
  Synthetic primer
aatttctagaatggccctgt

SEQ ID NO: 29 - DNA, 23 bp, Artificial Sequence
  Synthetic primer
aattctcgagctagttgcagtag

SEQ ID NO: 30 - protein, 9 aa, Artificial Sequence
  Synthetic peptide
LYLVCGERG

SEQ ID NO: 31 - protein, 9 aa, Artificial Sequence
  Synthetic pepptide
KYQAVTTTL

SEQ ID NO: 32 - DNA, 20 bp, Artificial Sequence
  Synthetic primer
accagtgtagagttgacgtg

SEQ ID NO: 33 - DNA, 20 bp, Artificial Sequence
  Synthetic primer
tatggctactgctgtcaggt

SEQ ID NO: 34 - DNA, 20 bp, Artificial Sequence
  Synthetic primer
gctactacgtctgctacaca

SEQ ID NO: 35 - DNA, 20 bp, Artificial Sequence
  Synthetic primer
tcaaataccatctgtgctgg

SEQ ID NO: 36 - DNA, 20 bp, Artificial Sequence
  Synthetic primer
agagggagctgacttattgc

SEQ ID NO: 37 - DNA, 20 bp, Artificial Sequence
  Synthetic primer
gggatctacaacttgtacgg

SEQ ID NO: 38 - DNA, 20 bp, Artificial Sequence
  Synthetic primer
tcacactgccagcgtaatgt

SEQ ID NO: 39 - DNA, 20 bp, Artificial Sequence
  Synthetic primer
ctgtggtaagcatgctctgt

SEQ ID NO: 40 - DNA, 20 bp, Artificial Sequence
  Synthetic primer
cacagtagagtgtcgcatgt

SEQ ID NO: 41 - DNA, 20 bp, Artificial Sequence
  Synthetic primer
ttcatggttctggtggctct

SEQ ID NO: 42 - DNA, 20 bp, Artificial Sequence
  Synthetic primer
gagcggttccatatgtgtct

SEQ ID NO: 43 - DNA, 20 bp, Artificial Sequence
  Synthetic primer
tgtatcagctcttccacctg

SEQ ID NO: 44 - DNA, 20 bp, Artificial Sequence
  Synthetic primer
agatgcatccatccacctaa

SEQ ID NO: 45 - DNA, 20 bp, Artificial Sequence
  Synthetic primer
tgcactcttccatgttacag

SEQ ID NO: 46 - DNA, 20 bp, Artificial Sequence
  Synthetic primer
cgatgtgagactgtccactt

SEQ ID NO: 47 - DNA, 20 bp, Artificial Sequence
  Synthetic primer
accatccatgacatgaaccg

SEQ ID NO: 48 - DNA, 20 bp, Artificial Sequence
  Synthetic primer
caatcatgttggacaactgc

SEQ ID NO: 49 - DNA, 20 bp, Artificial Sequence
  Synthetic primer
gctaccatgccaacttctgt

SEQ ID NO: 50 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 51 - DNA, 9048 bp, Homo sapiens
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacatacaaacaaaaaaaaaaaaaaa

SEQ ID NO: 52 - DNA, 1035 bp, Artificial Sequence; identical to SEQ ID NO: 1
  Synthetic nucleic acid sequence encoding chimera

SEQ ID NO: 53 - protein, 341 aa, Artificial Sequence; identical to SEQ ID NO: 2
  Synthetic chimeric protein

SEQ ID NO: 54 - protein, 1683 aa, Artificial Sequence
  Synthetic chimeric proteins FVIIIHSQ-Fc
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKSEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 10 aa, Artificial Sequence
  Synthetic peptide
DWEYSVWLSN

(19) United States
(12) Patent Application Publication
(10) Pub. No.: US 2017/0072032 A1
(43) Pub. Date: Mar. 16, 2017

(54) PRENATAL THERAPY TO INDUCE IMMUNE TOLERANCE

(71) Applicant: INSERM (Institut National de la Santé et de la Recherche Médicale), Paris (FR)

(72) Inventors: Roberto Mallone, Paris (FR); Sebastien Lacroix-Desmazes, Paris (FR)

(21) Appl. No.: 15/190,272

(22) Filed: Jun. 23, 2016

Related U.S. Application Data
(60) Provisional application No. 62/188,004, filed on Jul. 2, 2015.

(51) Int. Cl. A61K 39/00; C12N 7/00; A61K 39/145

(52) U.S. Cl. CPC A61K 39/0008; A61K 39/145; C12N 7/00; C12N 2760/16034; A61K 2039/55516

(57) ABSTRACT

Constructs and methods for inducing immune tolerance during gestation, e.g. in utero, are provided. The constructs comprise a moiety that targets and binds to the neonatal Fc receptor (FcRn) and a moiety comprising an antigen of interest for which immune tolerance is desired. Administration of the constructs to a fetus during gestation results in immune tolerance, e.g. to antigens that otherwise elicit an unwanted immune response such as an autoimmune reaction.

### SEQUENCE LISTING

This application includes as the Sequence Listing the complete contents of the accompanying text file “Sequence.txt”, created Jul. 1, 2015, containing 65,642 bytes, hereby incorporated by reference.

### BACKGROUND OF THE INVENTION

Field of the Invention

The invention generally relates to constructs and methods for inducing immune tolerance during gestation, e.g. in utero. In particular, the invention provides constructs comprising a moiety that targets the neonatal Fc receptor (FcRn) and a moiety comprising an antigen (Ag) of interest for which immune tolerance is desired, as well as methods of inducing immune tolerance to the Ag of interest by administering the constructs transplacentally.

Background of the Invention

Immune or immunological tolerance describes a state of unresponsiveness of the immune system to substances or tissues that otherwise have the capacity to elicit an immune response Immune tolerance is essential for normal physiology, with tolerance to “self” antigens being of particular importance in preventing the development of autoimmune diseases.

A developing fetus must actively learn to tolerate benign Ags such as those in or on its own cells (“self” Ags), as well as those on chimeric maternal cells that reside in fetal tissues, and environmental and food Ags that are transferred across the placenta during gestation. Central tolerance, induced in the thymus, is a key process by which the immune system learns to discriminate self from non-self, and peripheral tolerance, induced in other tissues and lymph nodes, provides a second fail-safe mechanism to prevent over-reactivity of the immune system to various self or environmental entities (allergens, gut microbes, etc.).

The mechanisms by which these forms of tolerance are established are distinct, but the resulting effect is similar. Central tolerance is achieved mainly by deletion of autoreactive conventional T cell clones (both CD4(+) and CD8(+)) and positive selection of natural regulatory T cells (nTregs) and is further supported by the suppressive influence of inducible CD4(+) CD25(+) FoxP3(+) Tregs (iTregs) generated by the peripheral response. Fetal CD4(+) T cells have a strong predisposition to differentiate into tolerogenic Tregs that actively promote self-tolerance. As the fetus nears birth, a transition necessarily occurs between the tolerogenic fetal immune system and a more defensive adult-type immune system that is able to combat pathogens.

Deficits in central or peripheral tolerance cause autoimmune disease, some of which are extremely debilitating and even life-threatening. These include syndromes such as systemic lupus erythematosus, rheumatoid arthritis, type 1 diabetes, autoimmune polyendocrine syndromes, acquired hemophilia and immunodysregulation polyendocrinopathy enteropathy X-linked syndrome (IPEX). Moreover, central and peripheral tolerance defects contribute to asthma, allergy, inflammatory bowel disease and, possibly, development of anti-drug antibodies.

Indeed, unwanted immune responses towards self-antigens also develop in several non-autoimmune conditions, for example, following administration of protein therapeutics, thus leading to treatment failure (1). This is particularly daunting for replacement therapies in the context of genetic deficiencies, since immune tolerance to a native but defective self-protein does not necessarily extend to an administered therapeutic protein. In this context, modulation of the T-cell repertoire and induction of (Ag)-specific Tregs appears as a strategy of choice to prevent allo- or auto-immunity (2).

Numerous approaches have been attempted to promote Treg-mediated tolerance, such as cytokine therapy (3, 4) modulation of signal transduction (5, 6) and intra- or extra-thymic delivery of target Ags (7-9). In the case of exogenous Ags, the time and route of Ag administration are critical to achieving optimal Treg induction. Since tolerance to self is first established in the thymus during immune ontogeny, the fetal period appears as a favorable time window for manipulating central tolerance employing exogenous Ags (10, 11). Indeed, transplacental transfer of maternal allo-Ags induces Treg-mediated Ag-specific tolerance in neonates.

As an example, hemophilia A is an X-linked genetic deficiency in clotting factor VIII which causes increased bleeding. About 70% of the time it is inherited as a recessive trait, but around 30% of cases arise from spontaneous mutations. Mild hemophiliacs often manage their condition with desmopressin, which releases stored factor VIII from blood vessel walls/endothelial cells. However, severe hemophilia patients may require regular supplementation with intravenous recombinant or plasma concentrate factor VIII. This can be especially problematic in children, and an easily accessible intravenous port may have to be inserted to minimize frequent traumatic intravenous cannulation. However, there are risks involved with the use of such ports, the most worrisome being that of infection. Studies differ but some show an infection rate as high as 50%. Also, there are other studies that show a risk of clots forming at the tip of the catheter.

A particular therapeutic conundrum is the development of “inhibitory” antibodies against factor VIII due to frequent infusions, as the body recognizes the “normal form” factor VIII that is administered as foreign. Therefore, in these patients, factor VIII infusions are ineffective. There is a need in the art for new approaches to treating, or preferably preventing, the development of anti-factor VIII inhibitory antibodies during hemophilia treatment.

As a second example, diabetes mellitus type 1 (type 1 diabetes, T1D) is a form of diabetes that results from the autoimmune destruction of the insulin-producing beta cells in the pancreas. The subsequent lack of insulin leads to increased blood and urine glucose. People with T1D are currently treated by insulin, but such treatment greatly impairs the life quality of the patient and blood glucose levels can be difficult to regulate. Other forms of treatment include pancreas and islet transplantation. However, suitable donors may be difficult to identify, and the surgery and accompanying immunosuppression required may be more detrimental than continued insulin replacement therapy when weighed against the benefits of the procedure.

Intense research efforts are ongoing to develop immunotherapies aimed at blunting islet autoimmunity. Antigen (Ag)-specific immunotherapies are particularly attractive due to their selectivity and safety, but have met with limited success. Several attempts have focused on tolerogenic vaccination with β-cell Ags derived from preproinsulin (PPI), which is the target initiating the autoimmune cascade in non-obese diabetic (NOD) mice and likely also in humans. A recent clinical trial employing intranasal insulin in slow-onset T1D patients did not result in C-peptide preservation, despite successful induction of insulin-specific immune tolerance. These results suggest that the timing of intervention may be too late, and that the Ag spreading that follows early β-cell destruction leads to a diversification of autoimmune reactions beyond insulin, thus making tolerance restoration to this sole Ag insufficient. The same problem is encountered in prevention trials. Despite an absence of clinical disease, selection of at-risk patients based on positivity for multiple auto-antibodies (auto-Abs) underscores the presence of an autoimmune reaction that has already spread to several Ags. Prospective cohorts of genetically at-risk children further highlighted that β-cell autoimmunity initiates very early, possibly already during fetal life, as the median age at auto-Ab seroconversion is only 9-18 months.

There is an ongoing need to develop new constructs and strategies for transplacental delivery of therapeutic agents, including agents which induce immune tolerance, thereby preventing and/or attenuating unwanted immune responses.

### SUMMARY OF THE INVENTION

The present invention involves the use of chimeric constructs which are optimized for transplacental delivery. The constructs comprise a modified Fc moiety that retains the ability to bind to the FcRn, and a second moiety that is or comprises at least one antigen of interest.

The presence of the FcRn binding moiety allows the constructs to traverse the placenta so that the fetus is exposed to the antigenic moiety. The constructs thus exploit the physiological pathway by which maternal immunoglobulins are transferred to fetuses. In some aspects, the Fc modifications prevent Fc dimer formation so that the overall size of the construct is decreased and transfer across the placenta is facilitated. As a result, the fetus is exposed to higher concentrations of the construct (and hence the antigen of interest) than can be achieved using prior art techniques, making the present technology more efficacious than that of the prior art. Generally, the antigen of interest is also size-modified and/or modified to prevent (or attenuate) unwanted interactions with other biological molecules, thereby further reducing the overall size of the construct. For example, in some aspects, the antigen moiety is or comprises FVIII that is modified, for example, by eliminating its ability to bind to von Willebrand Factor (vWF). As a result, the construct does not interact with and is not sterically encumbered by vWF, and the construct is able to traverse the placenta more rapidly than would be possible without this modification

According to aspects of the invention, the subject's immune system is exposed to an antigen of interest by in utero administration of a chimeric construct comprising a neonatal Fc receptor (FcRn) targeting moiety and an antigen moiety comprising at least one epitope of interest. Uptake of the chimeric construct via the FcRn receptor results in entry of the construct into the subject's circulatory system and thus exposure of the nascent immune system to the antigen. Because exposure to the antigen occurs in utero, a long lasting tolerogenic immune response is elicited Immune tolerance to the antigen is thus established before birth, and enduring immune tolerance results.

The chimeric constructs described herein are advantageously designed to be of a size and functionality that readily crosses the placenta. For example, in some aspects, the chimera is designed to elicit immune tolerance to FVIII, and the antigenic moiety of the chimera is a mutant FVIII that is not capable of binding to, or exhibits attenuated binding to, von Willebrand Factor (vWF), thereby decreasing unwanted, superfluous interactions with vWF (which would otherwise increase the effective size of the chimera) and facilitating transport of the chimera across the placenta. In addition, in some aspects, the Fc portion of the chimera is modified, e.g. so as to be monomeric while still retaining its FcRn binding properties, further decreasing the overall size of the chimera and facilitating its transport across the placenta.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this invention to provide recombinant polypeptide constructs comprising i) a targeting moiety that binds neonatal Fc receptor (FcRn); and ii) an antigenic moiety comprising at least one antigen or antigenic determinant,wherein the antigenic moiety does not comprise a full-length FVIII with tyrosine at position 1680 or a segment of FVIII with tyrosine at position 1680. In some aspects, the at least one antigen is preproinsulin (PPI) or other pancreatic beta-cell antigen or an antigenic fragment thereof. In other aspects, the at least one antigen is FVIII that does not bind to, or exhibits reduced binding to, von Willebrand Factor (vWF); for example, when position 1680 of said FVIII is not tyrosine. In further aspects, the targeting moiety is selected from human Fc γ ¼; a portion of human Fc γ ¼ sufficient to permit binding of said recombinant polypeptide construct to said FcRn receptor; monomeric Fc γ; and a Fc heterodimer. In yet other aspects, the segment of FVIII is a B domain deleted factor VIII (BDD FVIII).

The invention also provides methods of eliciting immune tolerance to at least one antigen or antigenic determinant of interest in a subject in need thereof. The methods comprise administering to the subject a recombinant polypeptide construct comprising i) a targeting moiety that binds neonatal Fc receptor (FcRn); and ii) an antigenic moiety comprising at least one antigen or antigenic determinant, wherein the antigenic moiety does not comprise a full-length FVIII with tyrosine at position 1680 or a segment of FVIII with tyrosine at position 1680. The subject may be a fetus and administration may be performed in utero. For example, in some aspects, the step of administering is performed transplacentally by administering the recombinant polypeptide construct to the mother. In various aspects, the at least one antigen is preproinsulin (PPI) or other pancreatic beta-cell antigen or an antigenic fragment thereof. In additional aspects, the at least one antigen is FVIII that does not bind to, or exhibits reduced binding to, von Willebrand Factor (vWF), such as when position 1680 of said FVIII is not tyrosine. In additional aspects, the targeting moiety is selected from human Fc γ ¼; a portion of human Fc γ ¼ sufficient to permit binding of said recombinant polypeptide construct to said FcRn receptor; monomeric Fc y ; and a Fc heterodimer. In addition, the segment of FVIII may be a B domain deleted factor VIII (BDD FVIII).

The invention also provides methods of eliciting immune tolerance to an antigen of interest in an offspring of a female, comprising, during gestation of the offspring by the female, administering to the female a recombinant polypeptide construct comprising i) a targeting moiety that binds neonatal Fc receptor (FcRn); and ii) an antigenic moiety comprising at least one antigen or antigenic determinant, wherein the antigenic moiety does not comprise a full-length FVIII with tyrosine at position 1680 or a segment of FVIII with tyrosine at position 1680.

In additional aspects, the invention provides methods of inducing an increase of thymic and/or peripherally derived regulatory T cells (Tregs) and/or a decrease in conventional T cells specific for an antigen of interest in a fetus, comprising delivering to the fetus a recombinant polypeptide construct comprising i) a targeting moiety that binds neonatal Fc receptor (FcRn); and ii) an antigenic moiety comprising at least one antigen or antigenic determinant, wherein the antigenic moiety does not comprise a full-length FVIII with tyrosine at position 1680 or a segment of FVIII with tyrosine at position 1680.

### BRIEF DESCRIPTION OF THE DRAWINGS

FIG. 1A-E. Schematic representation of Fc fusion constructs. (A, B) The selected domains of Influenza A virus (A: HA1) and of FVIII (B: A2 and C2) are shown. (C, D, E) The complete construct maps for the HA1Fc (C), A2Fc (D) and C2Fc fusion proteins (E) are depicted.

FIG. 2A-G. Transplacental transfer of antigenic HA1Fc from pregnant mice to fetuses is FcRn dependent. (A-B) In vivo imaging of pregnant WT (A) and FcRn−/− (B) mice after intravenous injection of HA1Fc- ALEXA FLUOR® 680 protein (100 μg) at E18. The panels show the fluorescence in mothers after 1 min (top) and in corresponding fetuses with placenta (bottom) after 4 h. (C-D) The fetuses from WT (red) and FcRn−/− (no fluorescence) mice after laser excitation at 4 and 24 h post-injection as in A-B. (E-F) Levels of HA1Fc (E) and HAI, (F) in plasma of pregnant mice (n=3) and fetuses (n=18), as measured by ELISA.

The optical density obtained with the mother's plasma 5 min after injection was set at 100%. The y-axes represent the levels of HA1Fc or HA1 as % relative to the starting levels in pregnant mice. (G) Proliferation of CellTrace Violet (CTV)-labeled splenic CD4⁺ T cells from HA-specific 6.5 TcR-Tg mice was analyzed in presence of mIgG1, HA₁₁₀₋₁₁₉ peptide or HA1Fc (0.06-1.6 μM, three-fold dilutions). The representative histogram shows proliferation in the presence of 0.06 μM of the three Ags. Percent proliferation at different Ag concentrations is shown on the right, as determined by gating on dividing 6.5 TcR-Tg CD4⁺ T cells based on CTV dilution. All-inclusive, results are representative of two to three independent experiments.

FIGS. 3A and B. Transplacental transfer of Fc-fusion proteins. (A-B) Plasma levels of HA1Fc (A) and C2Fc (B) in 18-24 fetuses (blood pooled from 3-6 fetuses) and 3 pregnant mice were determined by ELISA 4 hr after injection to pregnant wild-type and FcRn−/− mice. The y-axes represent the plasma levels of Fc-fusion proteins depicted in arbitrary units (AU).

FIG. 4A-F. Transplacentally transferred HA1Fc shapes the T-cell repertoire in HA-specific 6.5 TcR-Tg mice. (A) Representative contour plots describing the gating and total percent population of T cells in the spleen of 2-week-old mice treated transplacentally with HA1Fc or mIgG at E16, E17 and E18 of gestation. The CD4⁺ and CD8⁺ T-cell subsets are gated on CD3⁺ live T cells. TcR-Tg cells were identified using the 6.5 clonotypic antibody. Tregs were identified as CD3⁺CD4⁺CD25⁺Foxp3⁺ cells. (B-C) TcR-Tg (6.5⁺) and non-Tg (6.5⁻) T-cell subsets in spleens (B) and thymi (C) of 2-week-old mice treated transplacentally with HA1Fc (full circles) or mIgG (empty circle). (D-E) Graphs depicting TcR-Tg or non-Tg natural Tregs (nTregs; Nrp-1^{hi}) and induced Tregs (iTregs; Nrp-1^{lo}) in spleens (D) and thymi (E) of 2-week-old transplacentally treated mice. (F) Proliferation of splenocytes from 2-week-old mice treated transplacentally with HA1Fc (full circles) or mIgG (empty circle). Splenocytes were stimulated with either HA₁₁₀₋₁₁₉ peptide (left panel) or concanavalin A (Con A, right panel). The y-axes denote the proliferation index, calculated as the ratio of incorporated [³H]-thymidine in stimulated vs. non-stimulated cells. Results depicted in (A-F) are representative of four independent experiments. Data are means±SEM for 12 to 16 mice in each group. Statistical significance was assessed using two-sided Mann-Whitney U-test (A-E) or two-way ANOVA with Bonferroni post-test correction (F). ns, not significant.

FIG. 5. Transplacentally transferred HA1Fc is endocytosed by fetal myeloid cells. Pregnant mice were injected with either HA1Fc-Alexa Fluor 647 or PBS on E18. Fetuses were removed 24 hr after treatment to collect thymi (top row) spleens (middle row), and blood (bottom row). Single cell suspensions were obtained by pooling spleens, thymi or blood from at least 3 fetuses. The histograms show the indicated cellular subsets in fetuses from pregnant mice injected with either HA1Fc-ALEXA FLUOR® 647 (black lines) or PBS (grey profiles). The y-axes depict cell counts and x-axes show the HA1Fc-ALEXA FLUOR® 647 fluorescence. Percentages of HA1Fc- ALEXA FLUOR® 647⁺ cells (means±SEM of three independent experiments, each including 16-20 fetuses) are shown among live, CD11b⁺CD11c⁺SIRP-α⁺ cells (circulating DCs); CD11b⁺CD11c⁺SIRP-α⁻ cells (thymic resident DCs); CD11b⁺CD11c⁻F4/80⁺ cells (macrophages); CD11b⁻CD45R/B220⁺ cells (B cells); splenic T cells (CD3⁺TcRVβ8.1/8.2⁻); thymic CD4⁺ single positive (SP) T cells (CD3⁺CD4⁺CD8⁺).

FIG. 6A-F. Transplacental transfer of Fc-fused FVIII domains to the fetal circulation. (A) SPR affinity measurements. Real-time profiles of the binding of increasing concentrations (0.78 to 100 nM, two-fold dilutions) of A2Fc (top) and C2Fc (bottom) to immobilized recombinant mouse FcRn at indicated pH. The lower panel shows the kinetics of the interaction at pH 5.4, 6.4 and 7.4; dashes (−) indicate that no binding was detected. (B)

The fluorescence imaging of pregnant WT and FcRn−/− mice intravenously injected with C2Fc-ALEXA FLUOR® 680 protein (100 μg) at E18. The fluorescence images show the fetuses, connected to placenta, dissected from C2Fc-ALEXA FLUOR® 680-injected pregnant WT (top) and FcRn−/− (bottom) mice. (C-D) The fluorescence images show fetuses from WT (dark gray) and FcRn−/− (no fluorescence) mice after laser excitation at the indicated time points, treated as in B. Results are representative of 3 pregnant mice in each group, each carrying 6 to 10 fetuses. (E-F) Plasma levels of A2Fc (E) and C2Fc (F) in 18 fetuses and 3 pregnant mice. The y-axes represent the plasma levels of Fc-fusion proteins after 4 hrs of injection in mothers, depicted in arbitrary units (AU). Results are representative of three independent experiments.

FIG. 7A-D. Induction of tolerance to FVIII in hemA mice. (A) Treatment regimens in the mouse model of hemophilia A. The indicated combinations of Fcyl-fusion proteins were injected into pregnant mice at E16, E17 and E18. The mice and their progeny were bled at the time of weaning. The progeny was further bled before (week 6, W6) and after (2.5 months, W10) of replacement therapy with full-length FVIII administered weekly between week 6 and 9 (1 IU/mouse). (B) Anti-C2 IgG plasma titers of mice transplacentally treated with mIgG1 (empty circles) or C2Fc (filled squares) after replacement therapy with therapeutic FVIII. The y-axis represents the arbitrary levels of anti-FVIII IgG expressed as mg/mL ESH8-equivalent. (C) Anti-FVIII IgG plasma titers of mice treated transplacentally with mIgG1 (empty circles), A2Fc (filled triangles), C2Fc (filled squares) or A2Fc+C2Fc (filled circles) after replacement therapy with therapeutic FVIII (1 IU/mouse). The y-axis represents the arbitrary levels of anti-FVIII IgG expressed as mg/mL mAb-6-equivalent (D) FVIII inhibitory plasma titers expressed as Bethesda units (BU)/mL of mice transplacentally treated with mIgG1 (empty circles) or A2Fc+C2Fc (filled circles). Results are depicted as means+SEM and are representative of three independent experiments. Statistical significance was calculated by two-tailed Mann-Whitney U-test (ns: not significant).

FIG. 8A-C. Functional assessment of Tregs generated by transplacental treatment in hemA mice. (A) Proliferation of splenocytes from mice treated transplacentally with mIgG1 (empty circles) or A2Fc+C2Fc (filled circles). Splenocytes were stimulated with either FVIII (left) or concanavalin A (Con A, right). The y-axes shows the proliferation index, calculated as in FIG. 3F. (B) Suppression of proliferation of responder CD4⁻CD25⁻ Teff cells from FVIII-primed mice (n=8), in presence of FVIII, co-cultured with different ratios of Tregs from mice treated transplacentally with mIgG1 (Tregs pooled from 22 mice) (empty bar) or A2Fc+C2Fc (Tregs pooled from 16 mice) (filled bar). Y-axis indicates the percent suppression of proliferation in responder CD4⁺CD25⁻ Teff cells. (C) Anti-FVIII IgG titers in mice challenged with FVIII (1 IU/mouse) after adoptively transferred with either PBS (empty squares) or 1×10⁶ Tregs from mice treated transplacentally with either mIgG1 (Tregs pooled from 32 mice) (empty circles) or A2Fc+C2Fc (Tregs pooled from 42 mice) (filled circles).

The y-axis shows FVIII-specific IgG titers determined as in FIG. 6C. Results are depicted as means±SEM and are representative of three (A) or two independent experiments (B, C), with 5 to 8 mice per group. Statistical significance: two-way ANOVA with Bonferroni post-test (A) or two-tailed unpaired t-test (B, C). ns, not significant.

FIG. 9A-C. Biochemical validation of PPI1-Fc and PPI2-Fc fusion proteins. (A) cDNA and amino acid sequence of PPI-Fc constructs. Each construct was inserted into the pFastBac 1 Baculovirus plasmid between XbaI and XhoI restriction sites. The sequences of PPI1-Fc are here depicted: encoding nucleic acid, SEQ ID NO: 1; polypeptide: SEQ ID NO: 2. (B) Reducing SDS-PAGE of purified PPI1-Fc (left) and immunoblot analysis using anti-insulin and anti-Fc Abs (right). Identical results were obtained for PPI2-Fc. (C) Affinity measurements of FcRn binding by surface plasmon resonance. Biotinylated FcRn resuspended in Tris buffer (100 mM Tris, 100 mM NaCl, 0.1% Tween-20, pH 5.4) was immobilized on sensor chips at 1,000 resonance units and two-fold dilutions (from 200 to 0.78 nM) of test proteins injected at 30 μl/min. Association and dissociation phases were monitored for 5 min at 25° C., subtracted for the binding to uncoated chips and analyzed with the BIAevaluation v4.1. Real-time interaction profiles are shown for the binding of increasing concentrations of PPI1-Fc (top row), PPI2-Fc (middle row) and IgG1 (rituximab; bottom row) to immobilized recombinant mouse FcRn (mFcRn, left) or human FcRn (hFcRn, right). The experimental curves are presented along with curves generated by fitting data to the Langmuir binding model with a drifting baseline. Binding intensities are expressed in resonance units (RU). Representative sensorgrams from one of two independent experiments are shown.

FIG. 10A-F. PPI-Fc is transplacentally transferred from pregnant mice to their fetuses via Fc-FcRn binding. (A) In vivo fluorescence imaging of PPI-Fc placental transfer. G9C8 pregnant mice were i.v. injected at El8 with 100 μg of either PPI-Fc (first column) or PPI (second column; both proteins labeled with AF680), followed by in vivo imaging after 1 min (first row; external view on the dorsal side) and 24 h (second row; uterine horns exposed). Third column, β₂m^{-/-} pregnant mice (devoid of functional FcRn) were injected with PPI-Fc as above The fourth column displays the corresponding optical images of PPI-Fc-injected animals. (B) Fluorescence and optical images of exposed fetuses 24 h post-injection. (C) Optical and fluorescence images of 7-day-old G9C8 newborns 9 d post-injection of either PPI-Fc or PPI into pregnant mothers as above. Results are representative of 3 independent experiments. (D) Serum PPI-Fc concentrations at the indicated time points after maternal PPI-Fc treatment (as above) in G9C8 (filled circles) and β₂m^{-/-} pregnant mice (empty circles) and their fetuses (filled and empty squares; pooled sera), as determined by ELISA. (E) Urine PPI-Fc concentrations following maternal PPI-Fc treatment as above in G9C8 (filled circles) and β₂m^{-/-} pregnant mice (empty circles). (F) Urine PPI concentrations following maternal PPI-Fc (filled circles) or PPI treatment (empty circles) in G9C8 pregnant mice. Data are mean values±SEM of two independent experiments.

FIG. 11A-E. Transplacentally delivered PPI-Fc primes G9C8 TCR-transgenic T-cells and protects from diabetes. (A-B) In vitro CFSE proliferation assays on splenocytes isolated from 7-wk-old G9C8 mice born from untreated females. BMDCs prepared from naive G9C8 mice were pulsed with 26 μM PPI-Fc, PPI, PPI_{B15-23} or left unpulsed, then matured with LPS prior to culture with CFSE-labeled splenocytes for 5 d. CFSE profiles are shown after gating on CD8⁺ (A) and CD4⁺ T-cells (B) and are representative of two independent experiments. The proliferation index is indicated for each profile, calculated as the total number of cells in all generations divided by the number of original parent cells using FlowJo X (TreeStar). (C) Diabetes incidence in the G9C8 offspring of mice i.v. injected at E16 with 100 μg PPI-Fc (black solid line), equimolar amounts of IgG1 (grey solid line), PPI (grey dashed line) or PBS alone (black dashed line). Diabetes was subsequently induced by immunization with PPI_{B15-23} peptide and CpG at 3.5 and 5.5 wk of age. ***p<0.0001 by log-rank Mantel-Cox test. (D, E) Splenocytes were isolated from the 7-wk-old non-diabetic offspring of PPI-Fc- (gray circles) and PBS-treated G9C8 females (white circles) after two immunizations with PPI_{B15-23} peptide and CpG as above. (D) Percent of spleen CD8⁺ (left) and CD4⁻ T-cells (right); *p=0.01 by Student's t-test. (E) Percent of CD44⁺ memory (left) and CD62L⁺CCD44⁻ naive cells (right) out of total spleen CD8⁺ T-cells; *p=0.02. Data in (D-E) are mean±SEM from two independent experiments.

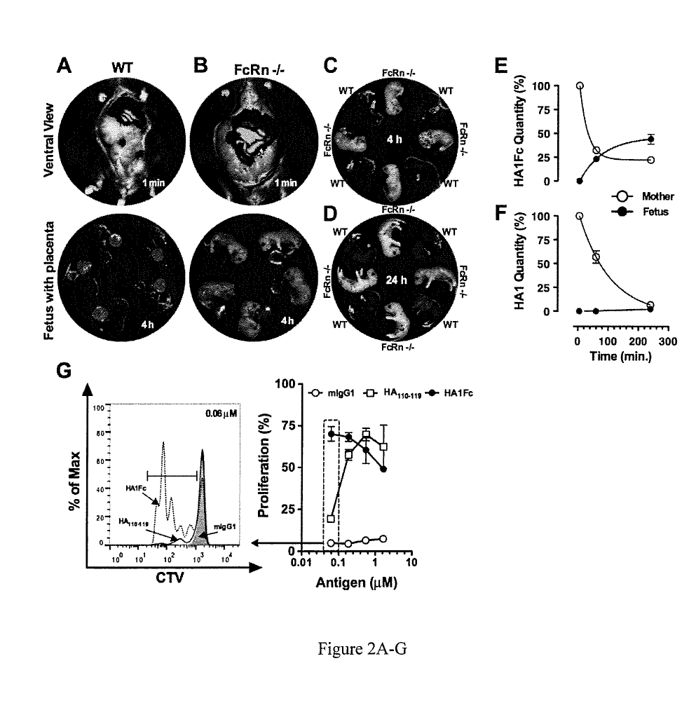

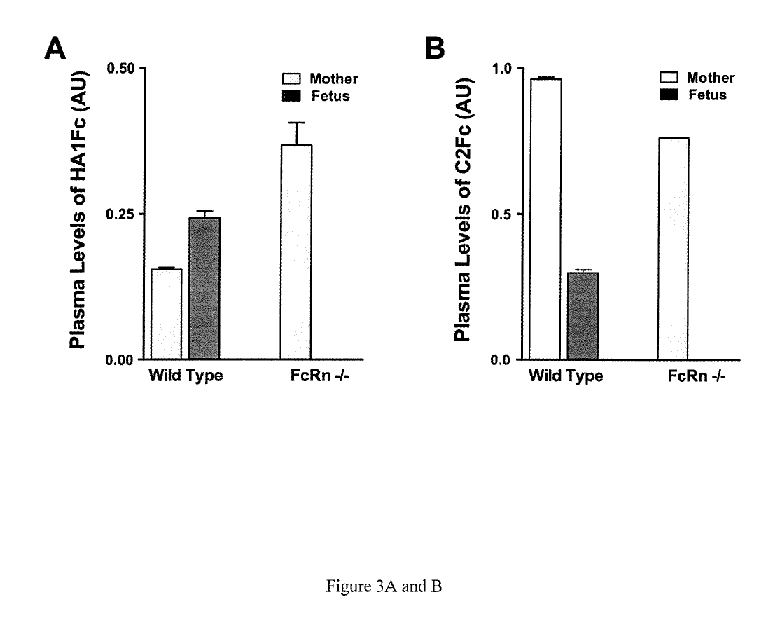

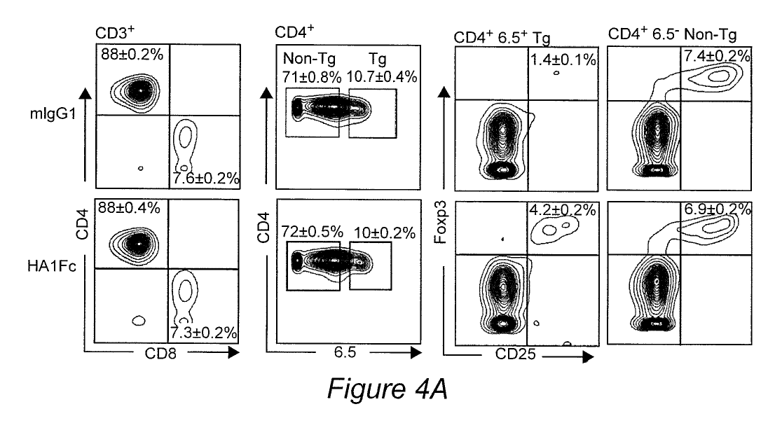

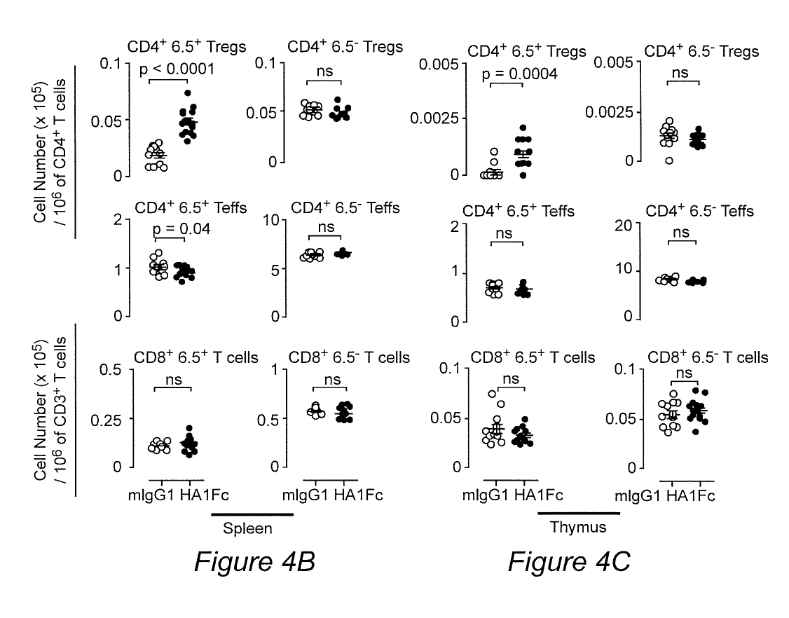

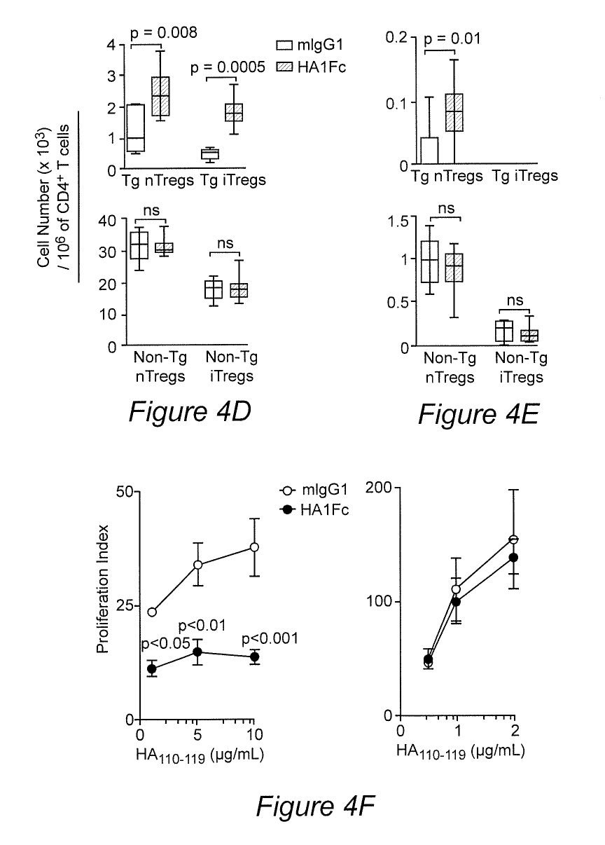

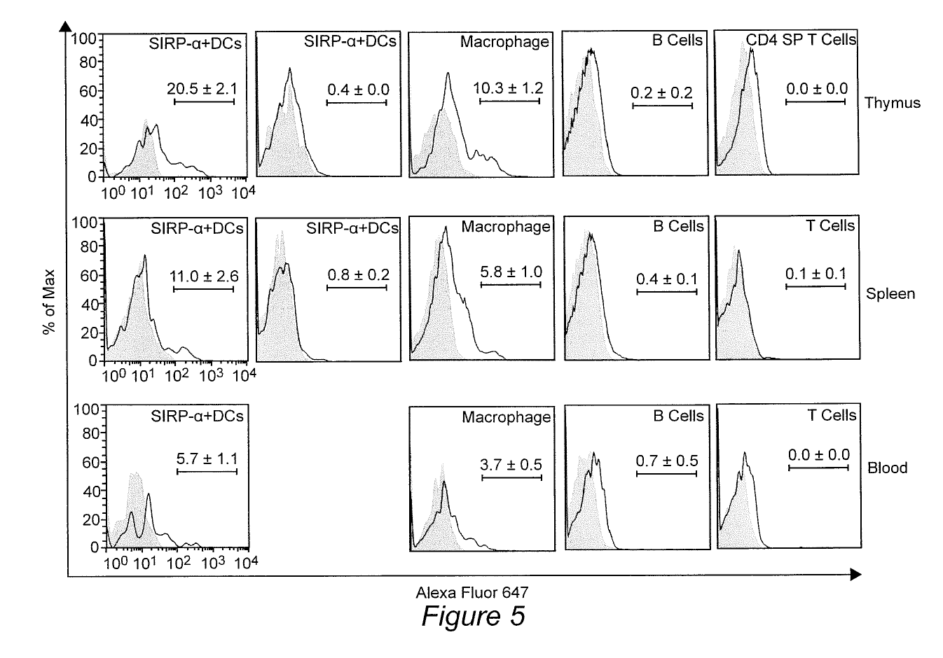

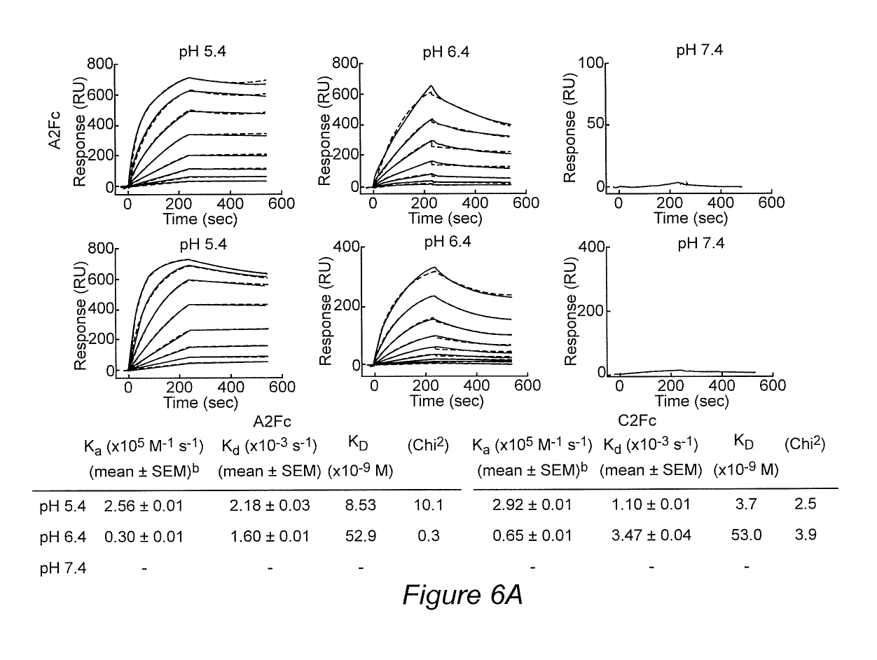

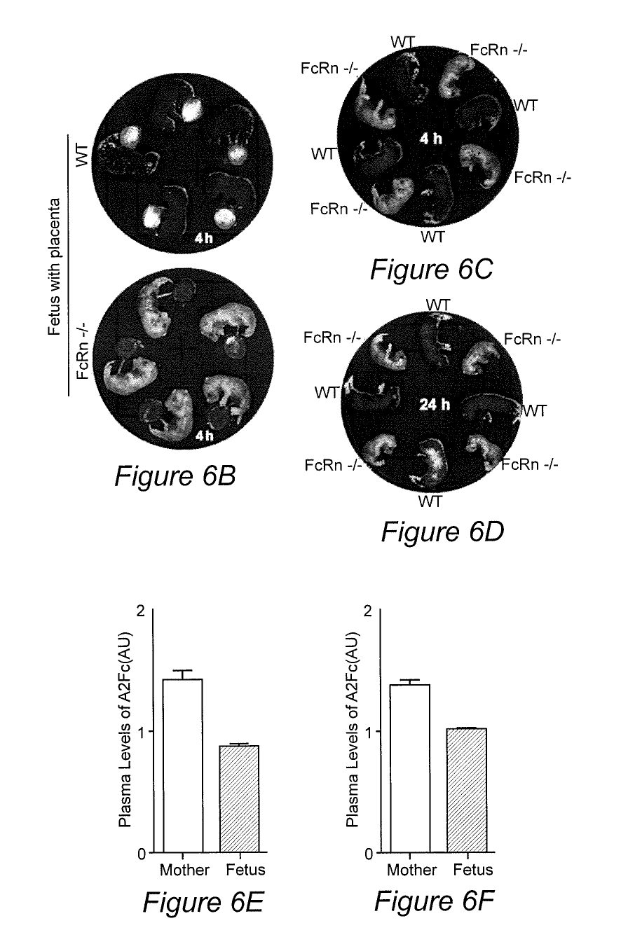

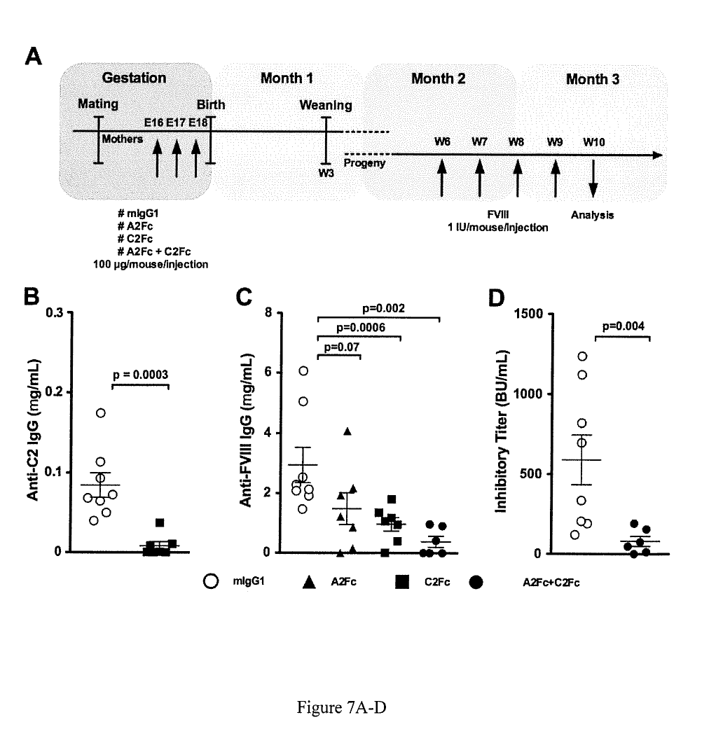

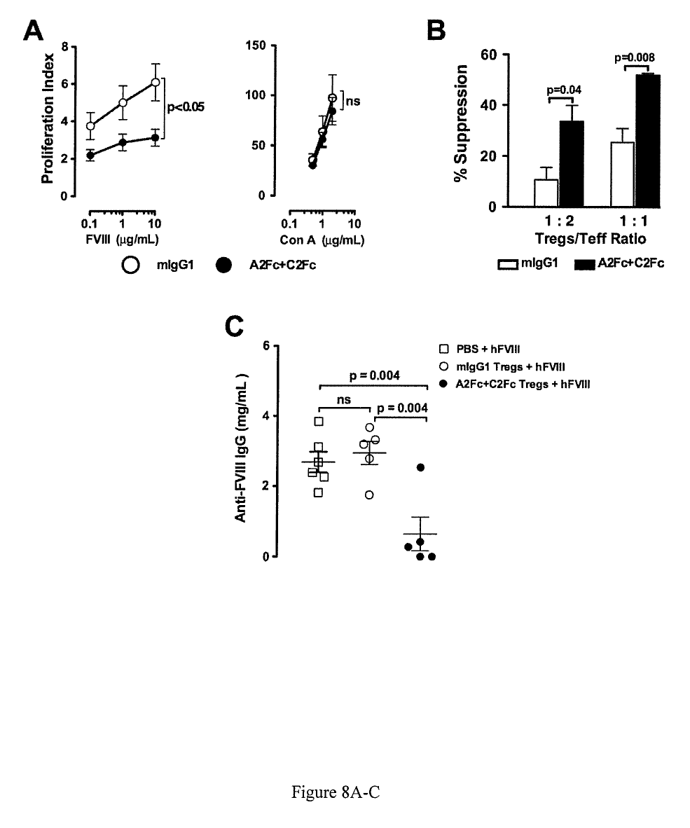

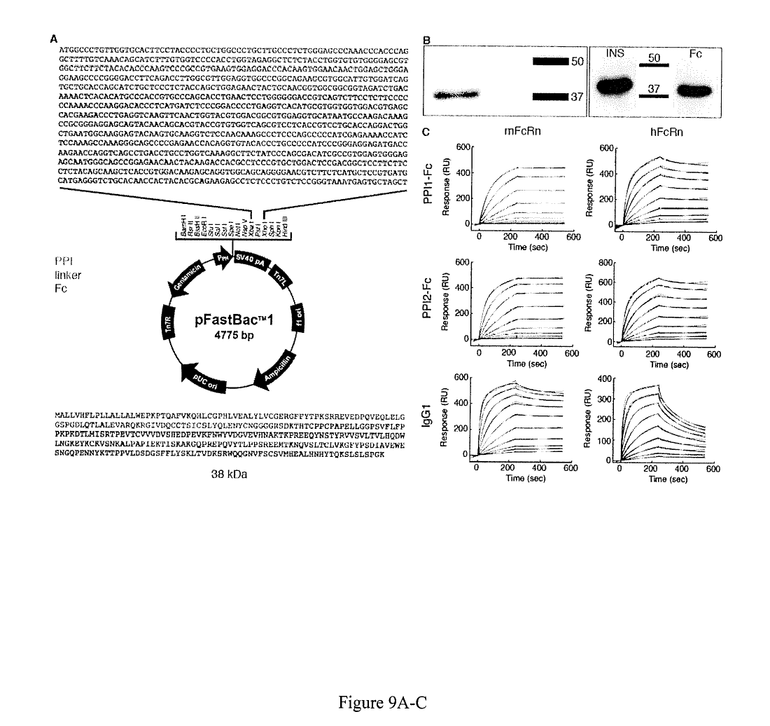

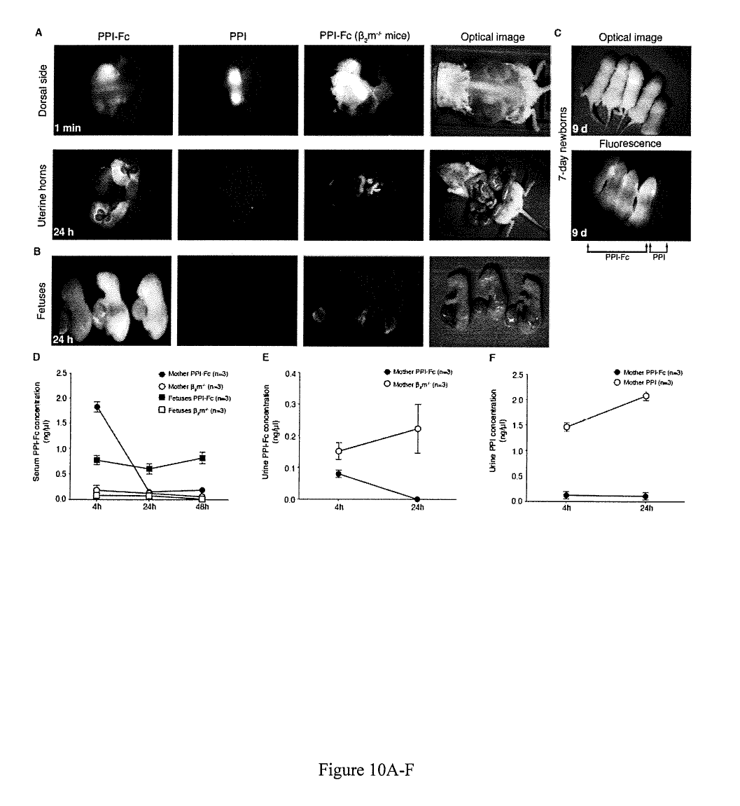

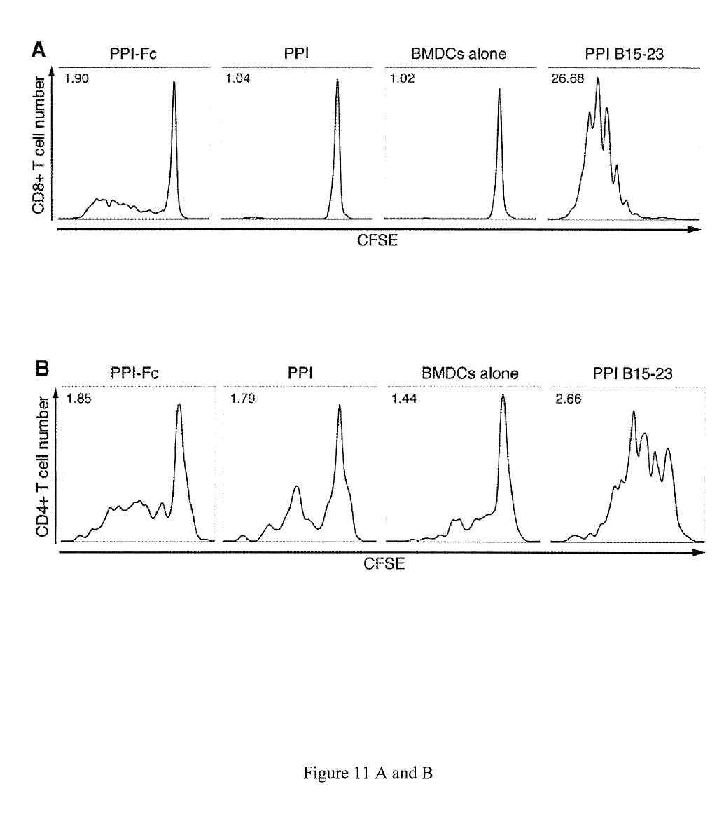

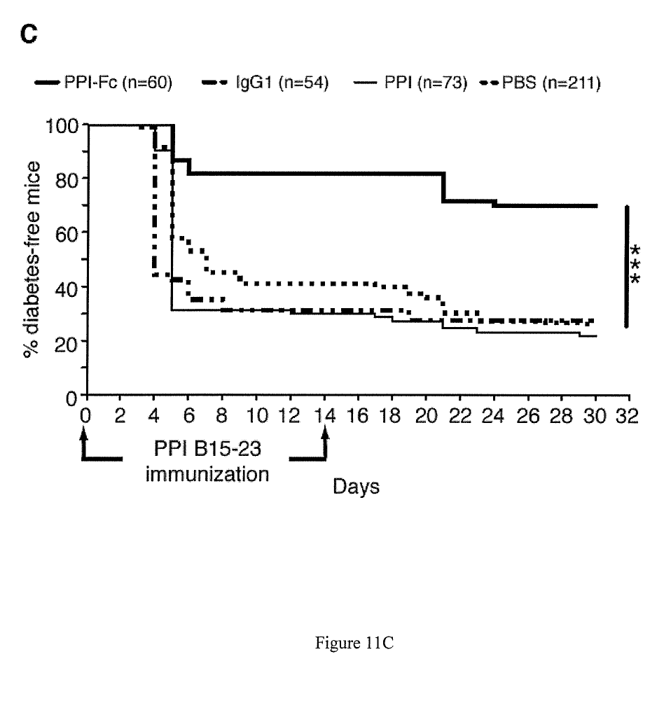

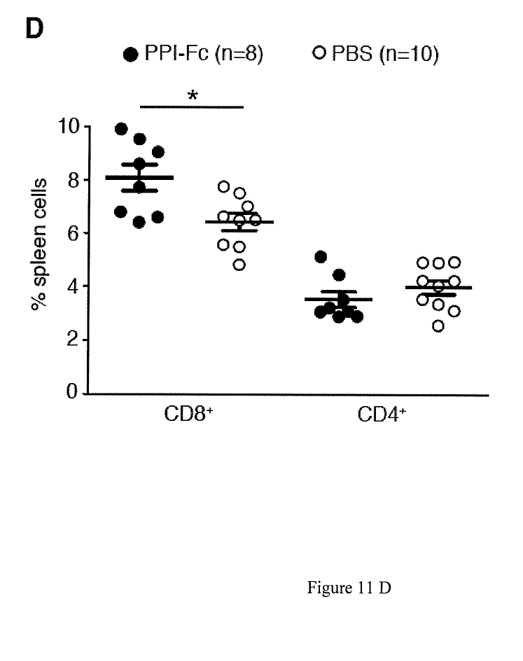

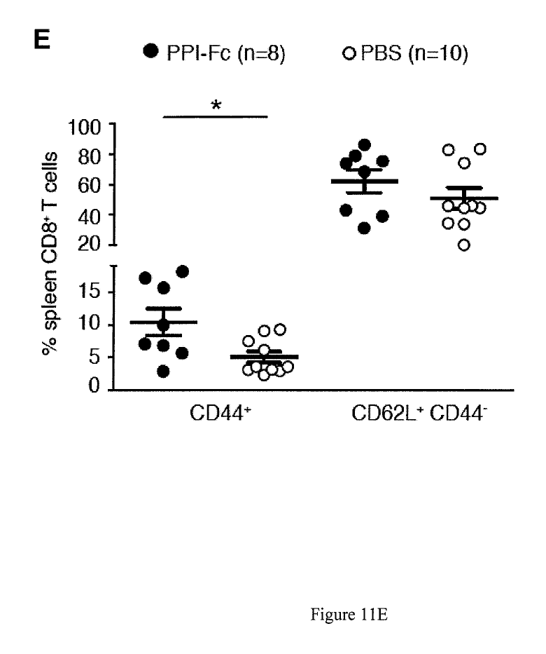

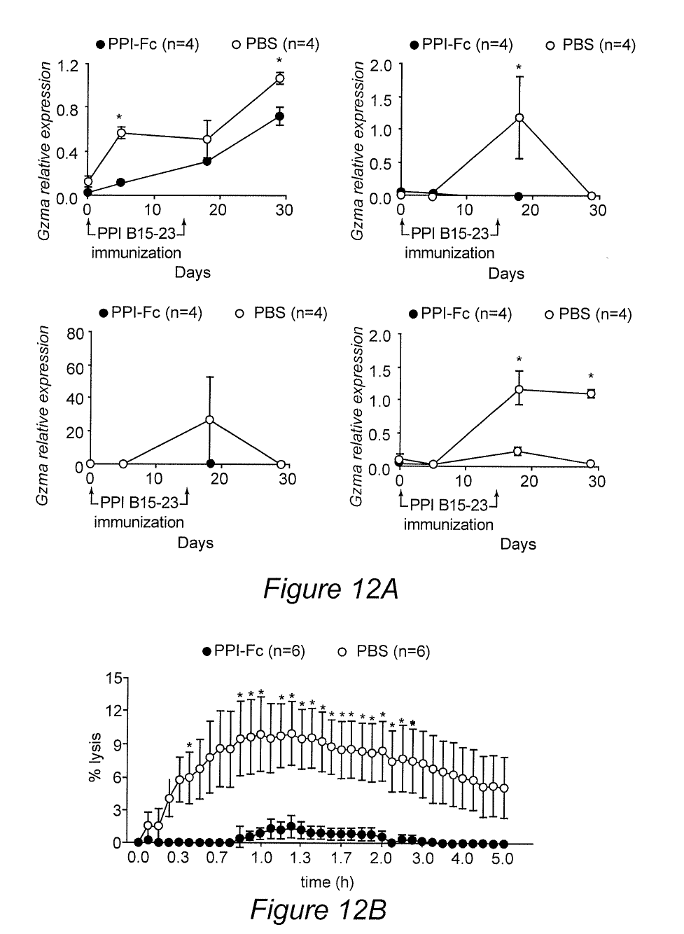

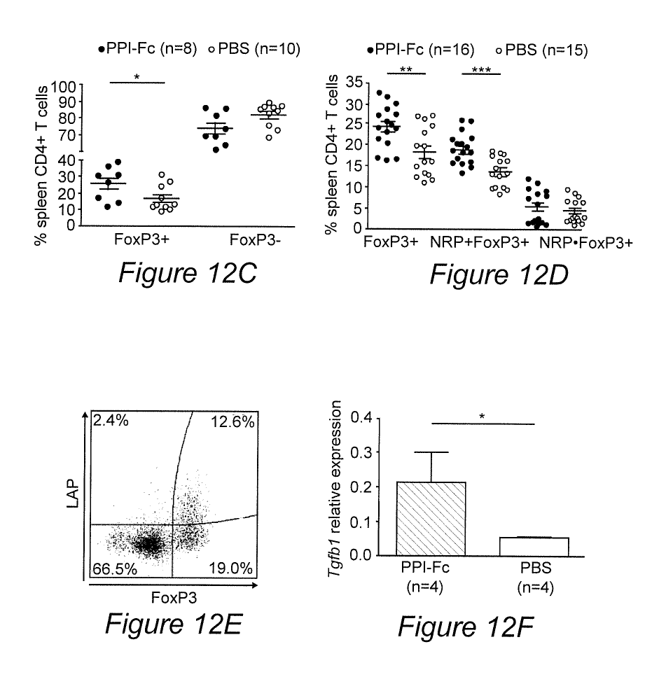

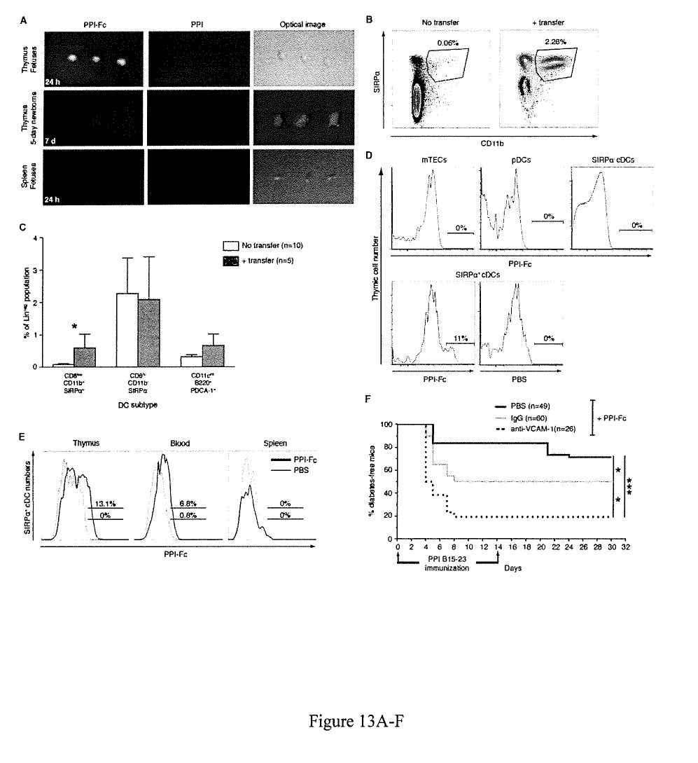

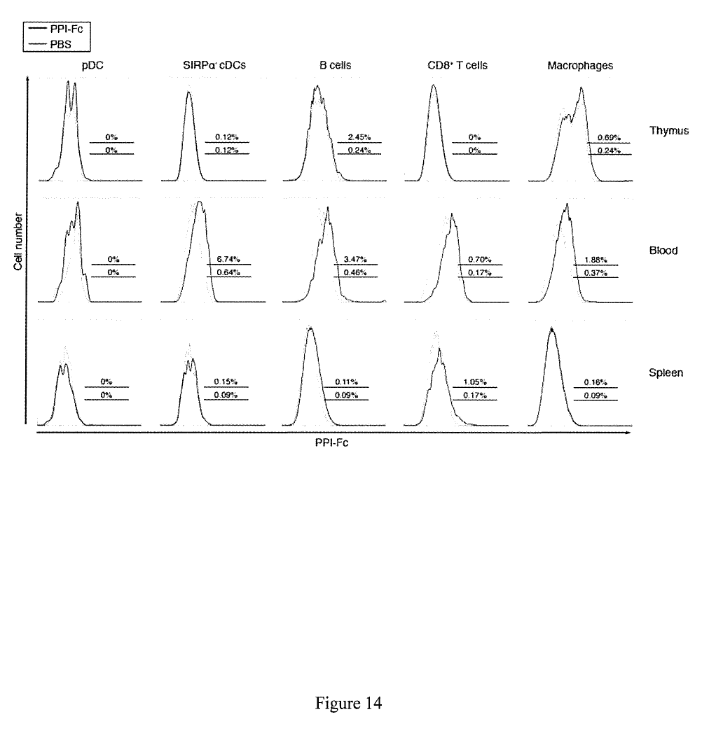

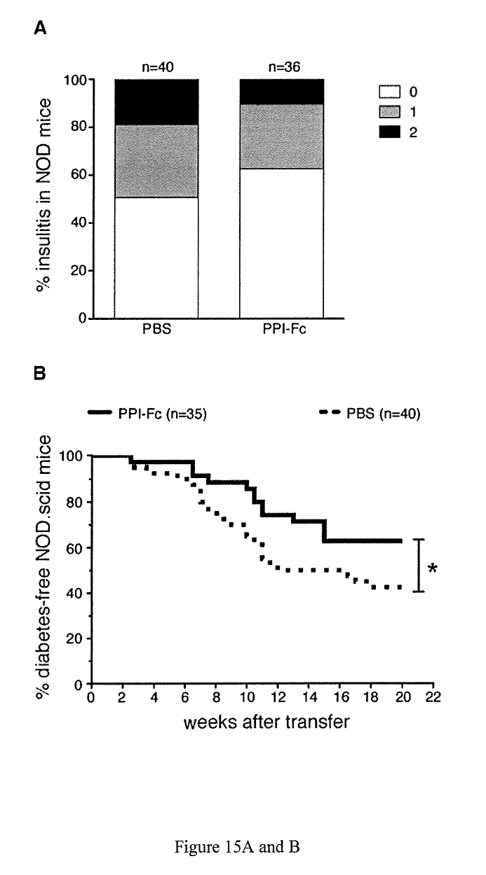

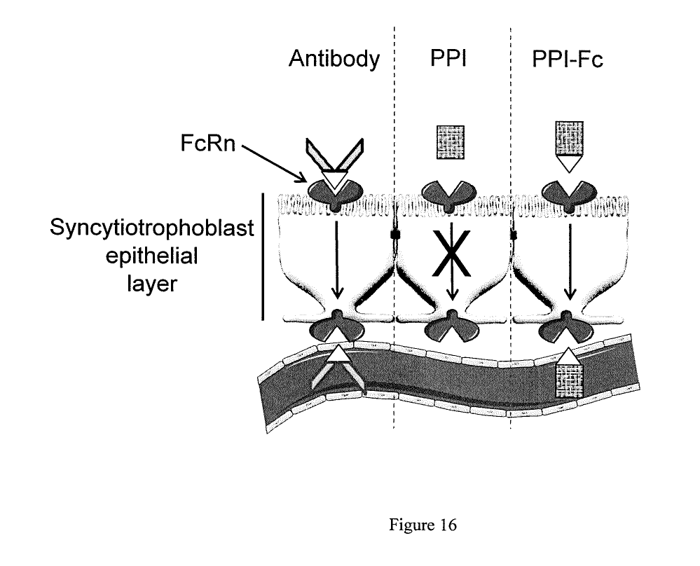

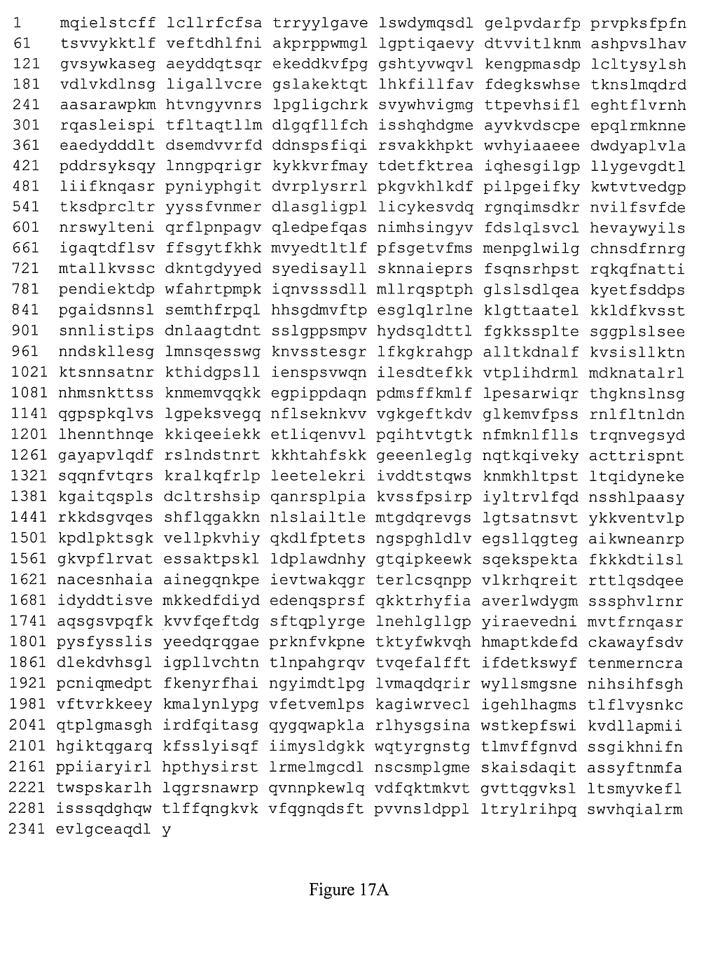

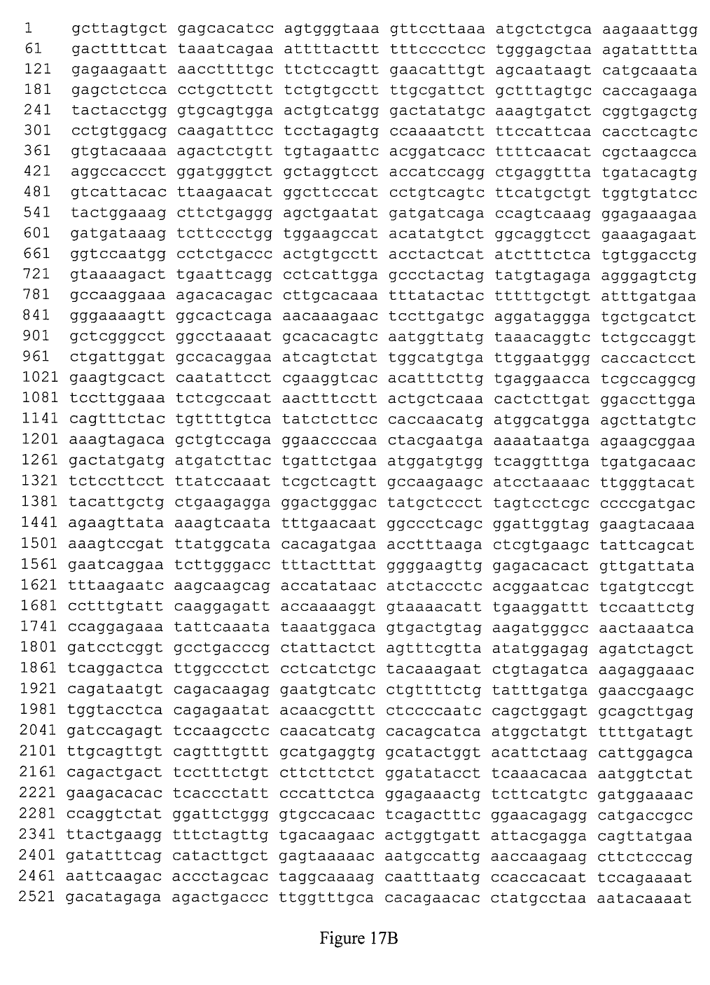

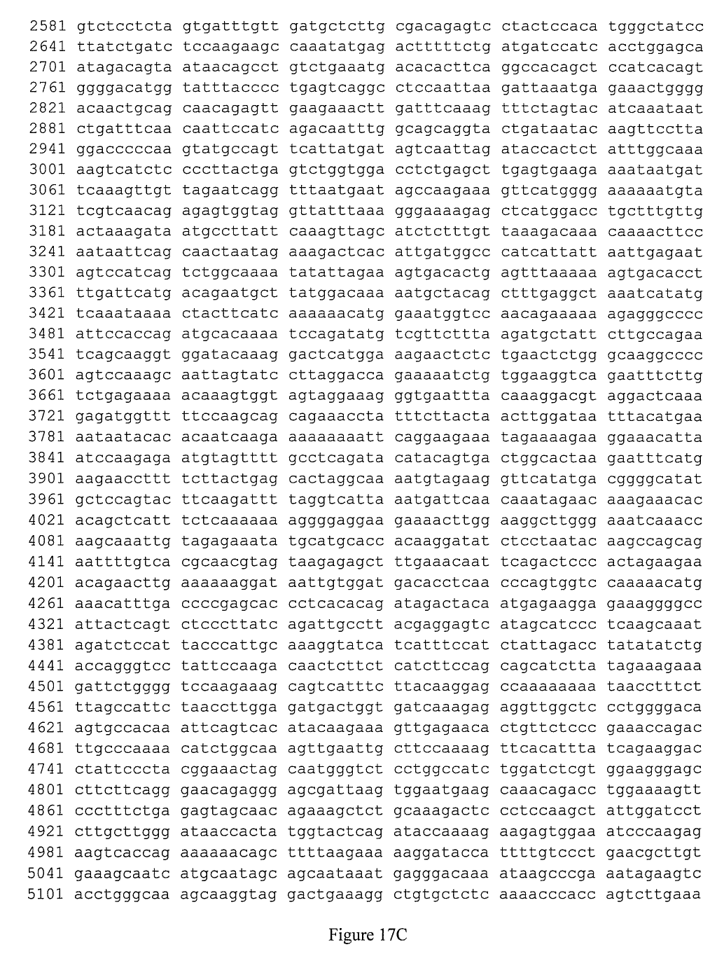

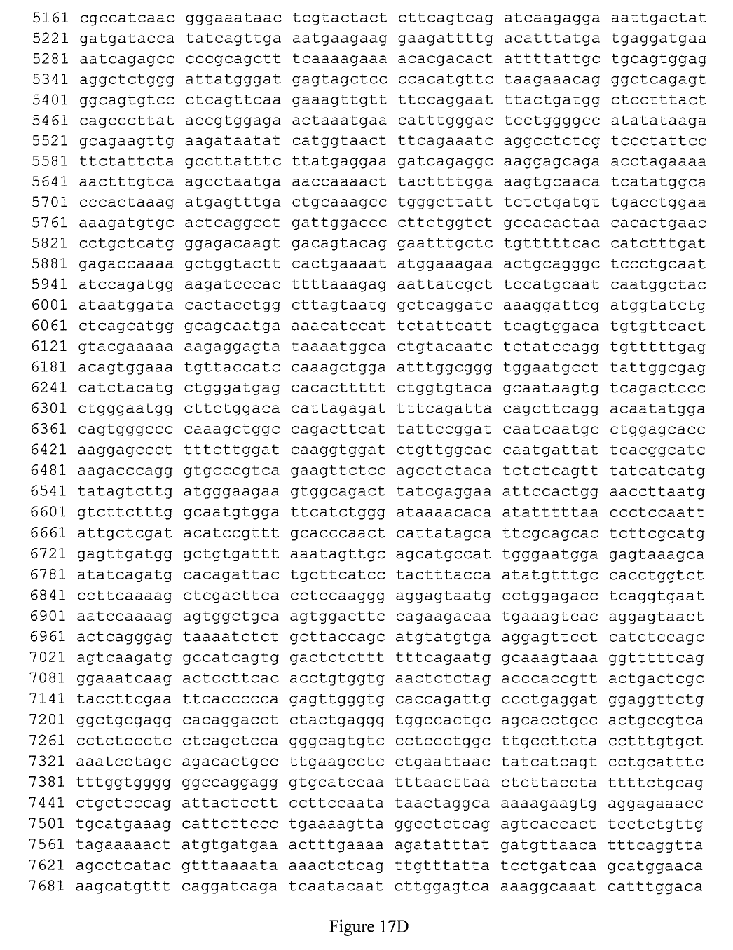

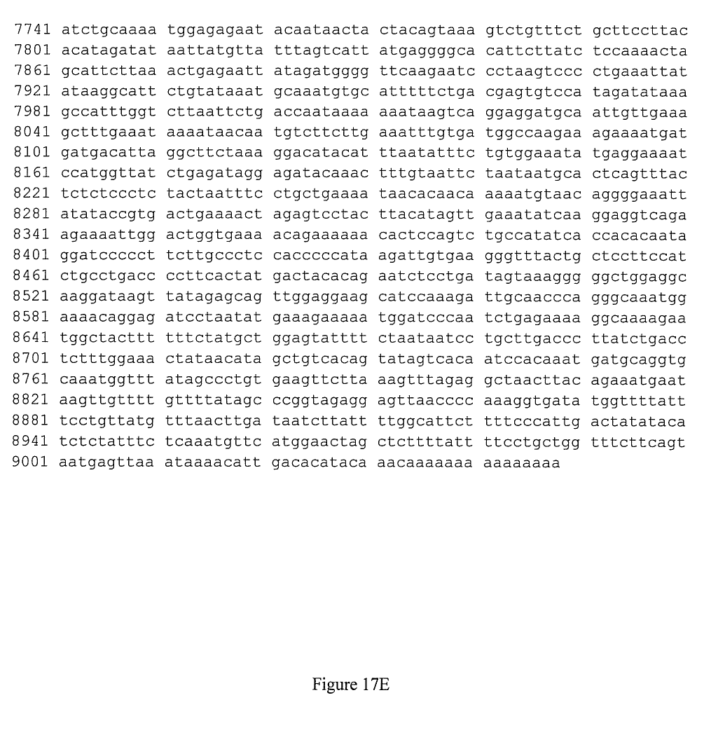

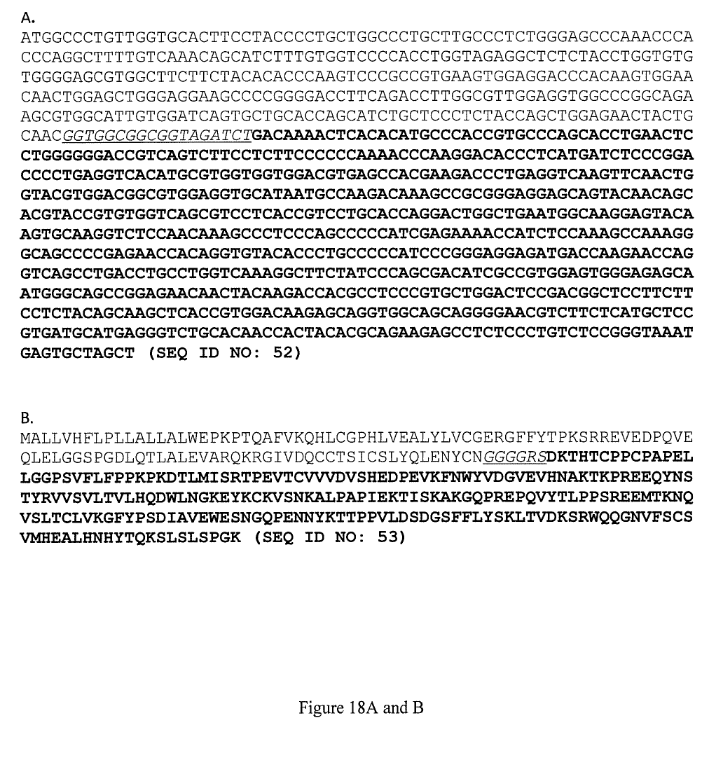

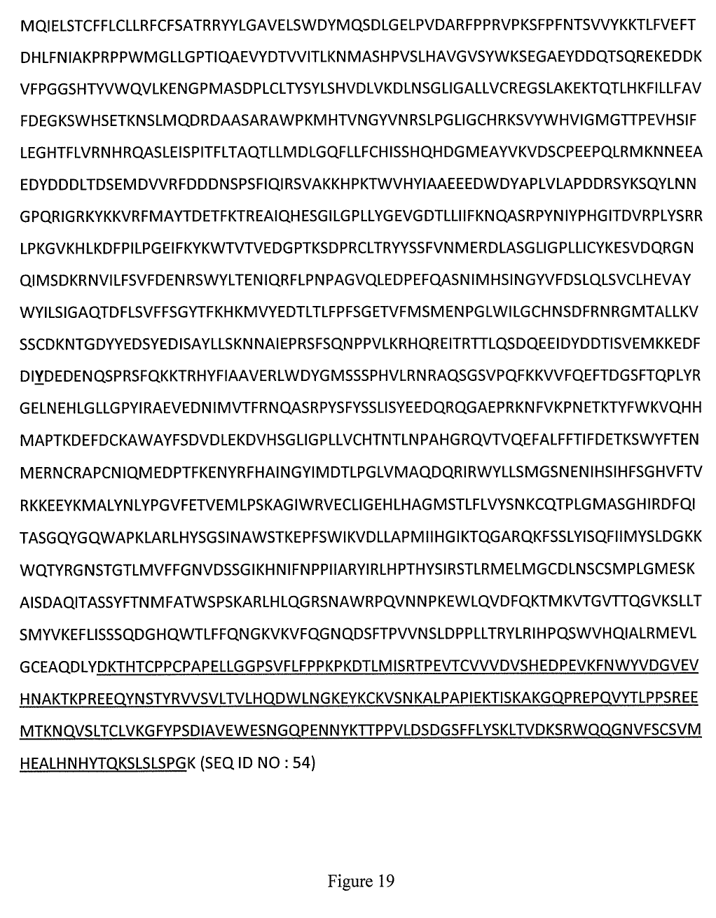

FIG. 12A-F. The offspring of PPI-Fc-treated G9C8 mice harbors CD8⁺ T-cells displaying impaired cytotoxicity and increased numbers of thymic-derived Tregs expressing TGF-β. (A) qRT-PCR expression profiles of the indicated genes in blood CD8⁺ T-cells sequentially obtained from G9C8 mice at the indicated time points, starting right before PPI_{B15-23} prime immunization (d 0); *p<0.03. (B) FACS-sorted CD8⁺ T-cells from the G9C8 offspring of mice i.v. injected at E16 with 100 μg PPI-Fc (black circles) or PBS alone (white circles) were tested in xCELLingence real-time cytotoxicity assays on K^{d-} mouse fibroblast L cells in the presence of 10 nM PPI_{B15-23} peptide. Mean±SEM values of triplicate measurements from 6 individual mice/group are shown at each indicated time point. xCELLingence cell indexes were normalized to values at the time of T-cell addition (t=0) and transformed into percent lysis values as follows: 100 ×(live targets cultured alone)−(live targets in the presence of T cells)/(live targets cultured alone). *p<0.05. (C) Percent of Foxp3⁺ (left) and Foxp3⁻ CD4⁺ T-cells (right) out of total spleen CD4⁺ T-cells in the 7-wk-old non-diabetic offspring of PPI-Fc- (gray circles) and PBS-treated G9C8 females (white circles) after two immunizations with PPI_{B15-23} peptide and CpG as above; *p=0.05. Splenocytes were isolated from the same mice as in FIG. 2D-E. (D) Percent of total Foxp3⁺ (left) and NRP1⁺Foxp3⁺ (middle) vs. NRP1⁻Foxp3⁻ CD4⁺ T-cell subsets (right) out of total spleen CD4⁺ T-cells, isolated as in (C); **p=0.005 and ***p=0.0003. (E) Representative Foxp3 and LAP staining of G9C8 splenocytes after a 24 h in vitro activation with plate-bound anti-CD3 (clone 145-2C11, 5 μg/ml) and IL-2 (Proleukin; 50 U/ml). Gate is on viable CD3⁺CD4⁺ T-cells and similar results were obtained with splenocytes from the offspring of PPI-Fc- and PBS-treated mice. (F) TGF-β gene expression in circulating CD4⁺ T-cells of 4-wk-old naïve G9C8 mice. *p=0.03. Data in A-D and F are mean±SEM from 2-3 independent experiments and statistical significance was calculated by Mann-Whitney U test.

FIG. 13A-F. Diabetes protection is dependent on ferrying of PPI-Fc to the thymus by migratory DCs. (A) Ex vivo fluorescence imaging of PPI-Fc accumulation in thymi. G9C8 pregnant mice were i.v. injected at E18 with 100 μg of either PPI-Fc (first column) or PPI (second column; both proteins labeled with AF680), followed by ex vivo imaging of thymi isolated from fetuses 24 h post-injection (first row) and from 5-day-old newborns 7 d post-injection (second row). Third row, imaging of fetal spleens 24 h after injection. The third column displays the corresponding optical images of PPI-Fc-injected animals. (B)

Representative staining of migratory cDCs (CD8^{low}CD11b⁺SIRPα⁺) in thymi isolated from 5-wk-old NOD.scid mice 24 h after i.v. transfer of total blood cells from 1-day-old G9C8 newborns (right), in comparison with non-transferred mice (left). (C) Percentage of migratory cDCs (CD8^{low}CD11b⁺SIRPα⁺), resident cDCs (CD8^{hi}CD11b⁻SIRPα⁻)and pDCs (CD11c^{int}B2201⁺PDCA-1⁺) in thymi of 5-wk-old NOD.scid mice 24 h after i.v. blood cell transfer as above. Mean±SEM values from two separate experiments are represented. *p=0.05 by Maim-Whitney U test. (D) PPI-Fc uptake by different thymic subsets. G9C8 pregnant mice were i.v. injected at E19 with 100 μg of either AF647-labeled PPI-Fc or 100 μl PBS. Thymi were isolated from newborns 24 h post-injection. (E) Flow cytometry analysis of migratory SIRPα⁺ cDCs in neonatal thymi, blood and spleens isolated 24 h after injection of AF647-labeled PPI-Fc (black) or PBS (grey profiles), as above. Percentages of PPI-Fc⁺ cells are shown after gating on SIRPα⁺ cDC cells and are representative of 3 independent experiments. The gating strategy used in (B-E) is detailed in Supplementary FIG. 3. (F) Diabetes incidence in the G9C8 offspring of PPI-Fc-injected mice pre-treated with an IgG isotype control or with anti-VCAM-1 mAb. Pregnant mice were i.v. injected at E15 with 100 μg IgG (grey line), 100 μg anti-VCAM-1 mAb (dashed line), or PBS (black line), followed 24 h later by 100 μg PPI-Fc. Diabetes was subsequently induced in their offspring by immunization with PPI_{B15-23} peptide and CpG at 3.5 and 5.5 wk of age as before. *p=0.01; ***p<0.0001 by log-rank Mantel-Cox test.

FIG. 14. PPI-Fc uptake by different cell subsets in neonatal thymi, blood and spleens. Pregnant G9C8 mice were intravenously injected at E19 with 100 μg PPI-Fc labeled with AF647 (dark grey) or PBS (light grey). Single-cell suspensions were obtained by pooling thymi, blood or spleens from at least 5 newborns sacrificed 24 h post-injection. Percentages of AF647⁺ cells are shown for thymic (top row), blood (middle row) and splenic (bottom row) cell subsets, namely pDCs (CD11c^{int}B220⁺PDCA-1⁺), SIRPα⁻cDCs (CD8^{hi}CD11b⁻SIRPα⁻), B-cells (CD3⁻CD11b⁻B220⁺), CD8⁺ T-cells (CD11c⁻CD3⁺CD8⁺) and macrophages (CD3⁻CD11c⁻CD11b⁺). Results are representative of 2 independent experiments.

FIGS. 15A and B. The offspring of PPI-Fc-treated NOD mice displays milder insulitis and less diabetogenic splenocytes. Pregnant NOD mice were i.v. injected at E16 with 200 μg PPI-Fc or PBS vehicle. Splenocytes from their 14-wk-old pre-diabetic female progeny were subsequently transferred into 4- to 6-wk-old NOD.scid mice (15×10⁶/mouse). (A) Insulitis score was evaluated in pancreatic islets from the NOD female progeny upon sacrifice for splenocyte isolation and transfer. An average of 50 islets per pancreas were scored in blind for mononuclear cell infiltration, as follows: 0, no infiltration (white; p=0.02); 1, peri-insulitis (grey); and 2, insulitis (covering >50% of the islet; black; p=0.001); p=0.007 for the average insulitis score between the two groups, as assessed by Student's t-test. (B) Diabetes incidence in NOD.scid mice following adoptive transfer of splenocytes from the progeny of PPI-Fc- (solid line) and PBS-treated NOD mice (dashed line). *p=0.04 by log-rank Mantel-Cox test.

FIG. 16. A, schematic illustration of selective binding by the FcRn receptor. Fc-Fused antigens such as preproinsulin (PPI) cross the epithelial barrier of the syncytiotrophoblast and are released in the circulation by binding to the neonatal FcR (FcRn).

FIG. 17A-E. Sequences pertaining to native human FVIII. A, amino acid sequence

(SEQ ID NO: 50) and B-E, nucleic acid sequence (SEQ ID NO: 51).

FIGS. 18A and B. A, nucleic acid sequence (SEQ ID NO: 52) and B, amino acid sequence (SEQ ID NO: 53), of PPI1-Fc construct. Normal font=PPI1; underlined italics=linker; bold font=hFc.

FIG. 19. Amino acid sequence of the FVIIIHSQ-Fc construct, and exemplary construct in which the B domain of FVIII is absent (SEQ ID NO: 54). Normal font=FVIII portion (minus B domain) in which residue 1680 (Tyr) is underlined and in bold; underline corresponds to the Fc portion of the construct.

### DETAILED DESCRIPTION

Placental transfer of maternal IgG antibodies to the fetus is an important mechanism that provides protection to the infant while his/her humoral response is inefficient. IgG is the only antibody class that significantly crosses the human placenta, with IgG1 and IgG4 being most efficiently transported, and IgG2 the least. The crossing is mediated by the neonatal Fc receptor (FcRn), which mediates uptake of the IgG into the fetal circulatory system. Maternal IgG in fetal circulation increases from the early second trimester to term, conferring passive is immunity against neonatal and infantile infectious diseases to newborns, and tolerance to “self” antigens is also initiated during this time period. The present invention harnesses the FcRn in utero delivery mechanism to deliver one or more antigens of interest to the fetal circulatory system, thereby exposing the developing fetal immune system to the one or more antigens of interest. Exposure to the antigens during this critical time period results in the development of lasting tolerance to the antigens, thereby preventing the post-gestational occurrence of unwanted immune reactions (e.g. such as an autoimmune disease). If a subject has or is suspected of having a predisposition to develop, or is somehow likely to develop, an immune response against a self antigen or an antigen administered after birth, according to an aspect of the invention, the antigen is delivered to the subject in utero as part of a chimeric construct which also includes an FcRn targeting moiety. The construct, and hence the antigen, enters the fetal circulatory system via the FcRn pathway. Early exposure to the antigen drives the immune system of the subject to recognize the antigen as an antigen to which an immune response should not be elicited. Immune tolerance to the antigen is thus established, and later in life the subject's immune system does not mount an immune response to the antigen when it is encountered.

The following definitions are used throughout:

An antibody (AB), or immunoglobulin (Ig), is a protein produced by the immune system in response to exposure to a specific antigen that the body recognizes as foreign (non-self). Thereafter, the antibodies that are produced in response to the antigen combine chemically the antigen and neutralize or inactivate it, or at least attempt to do so.

Antigen: a substance that can stimulate the immune system to produce antibodies and/or T cells against it, and which can combine specifically with the antibodies and/or T-cell receptors that are produced by this stimulation.

Immunoglobulin G (IgG) is a type of antibody and is composed of four peptide chains: two identical heavy chains and two identical light chains arranged in a Y-shape typical of antibody monomers.

A “fragment crystallizable” region (Fc region) is the tail or base of the Y region of an antibody. The Fc region interacts with cell surface receptors called Fc receptors (e.g. neonatal Fc receptor) and some proteins of the complement system.

Epitope (antigenic determinant): the part of an antigen molecule to which an antibody or T-cell receptor attaches itself.

Neonatal Fc receptor (FcRn): The neonatal Fc receptor is an Fc receptor which is expressed on syncytiotrophoblast cells of the embryonic placental villi that invade the wall of the uterus and establish nutrient circulation between the embryo and the mother. Two FcRn molecules bind to a single maternal IgG molecule with a 2:1 stoichiometry, and the receptors mediate uptake of the IgG into the fetal circulatory system.

FIG. 16 depicts a schematic representation of an aspect of the invention. In this figure, what is shown in the left panel is the normal route of an IgG antibody when crossing the syncytiotrophoblast layer and gaining access to the fetal circulatory system. This FcRn pathway physiologically delivers maternal IgG to the offspring through the placenta during fetal life and through the gut during lactation. As can be seen, the Fc portion of the IgG antibody molecule (represented by a triangle) binds to the FcRn receptor which is located in the syncytiotrophoblast layer (both on the plasma membrane, as depicted, and intracellularly, not shown). Binding to FcRn (which can occur either extracellularly or intracellularly) results in antibody release into the fetal circulation. The middle panel shows that an antigen on its own cannot bind to the FcRn receptor and thus cannot cross or can only poorly cross the epithelial layer. The right hand panel shows that, however, when the antigen is conjugated to an Fc moiety, the Fc moiety binds to the FcRn receptor and the complex is delivered to the fetal circulatory system. Thus, the antigen is “piggy-backed” into the fetal circulatory system by the Fc moiety of the construct. Antigen-Fc introduction, especially during the perinatal period, thus induces immune tolerance.

In some aspects, the constructs described herein are optimized for transplacental delivery to a fetus. For example, one or both of the following may be carried out: 1) reduction in size one or more construct components (e.g. one or both of the antigen and Fc components) to allow transplacental transfer of the Ag-Fc complex; and 2) abrogation of binding to Fc receptors other than FcRn.

The two approaches are not mutually exclusive, and for example, may involve:
1) modification of the antigen (i.e. FVIII) to prevent binding to partners in the blood (e.g. VWF); and/or
2) various modification of the Fc portion.

In some aspects, recombinant chimeric constructs comprising an FcRn targeting moiety and at least one antigen of interest are provided. The FcRn targeting moiety is typically a protein or polypeptide that is capable of binding to and mediating uptake of the entire construct by the FcRn. Generally, the FcRn targeting moiety is an Fc of an IgG antibody, or a portion of the Fc that is sufficient to permit binding and uptake (transport across the membrane) of the construct. Without being bound by theory, it appears that binding to the FcRn occurs mostly intracellularly, while the Ag is mostly delivered into the cell by non-receptor-mediated endocytosis. Exemplary Fc's or portions thereof that may be used in the practice of the invention include but are not limited to: human Fc gamma (Fcg) 1 or 4; monomeric Fcg1 or Fcg4; constant heavy chain e.g. monomeric CH3; complementary Fcg1 or Fcg4 that does not homodimerize; various Fc heterodimers; etc. US20140294821 (Dumont et al.) and US20150050278 (Dimitrov, et al.) describe exemplary Fc and CH3 moieties that may be used in aspects of the present invention as does US20140370035 (Jiang et al.); monomeric CH1-CH2-CH3, various Fc mutations; knob-into-hole approaches as described, for example, in U.S. Pat. No. 8,592,562 to Kannan et al.; U.S. Pat. Nos. 7,695,936, 8,216,805, and 8,679,785 to Carter et al. and US patent application 20120225071 to Klein et al., the complete contents of each of which are hereby incorporated by reference; etc. Any suitable version of Fc may be used, so long as the construct ultimately delivers the antigen portion of the construct to the fetal circulatory system.

The chimeric construct also comprises an antigenic or antigen-containing moiety. This moiety comprises an antigenic molecule or a portion of an antigenic molecule for which is it desired to generate immune tolerance, and may comprise a plurality of antigens or portions of antigens. The antigen may be, for example, a protein, polypeptide or peptide, or another type of antigen such as a nucleic acid (e.g. DNA, RNA etc.), a lipid, a saccharide or polysaccharide, etc. The antigen moiety may contain one or more known epitopes of interest, e.g. regions or residues of the antigen which are known to elicit an immune response. Alternatively, putative epitopes and antigenic regions may be selected based on a likelihood of antigenicity due to accessibility, surface exposure, charge, amino acid sequence etc. e.g. using prediction software programs such as: for proteins of known 3D structure solvent accessibility can be determined using a variety of programs such as DSSP, NACESS or WHATIF, among others. If the 3D structure is not known, the following web servers may be used to predict accessibilities: PHD, JPRED, PredAcc (c) and ACCpro. N-glycosylation sites can be detected using Scanprosite, or NetNGlyc. Various programs for prediction include the website located at www.iedb.org, those developed by DNASTAR, the OPTIMUN ANTIGEN™ Design Tool, and others. Other web-based algorithms to predict immunogenicity, particularly immunogenicity related to the elicitation of T-cell responses, include, but are not limited to, SYFPEITHI, HLA-Restrictor, NetMHC and IEDB.

If the antigen is a protein or polypeptide, an antigenic region that is present in the antigenic moiety may be contiguous sequences of the primary sequence of an antigen. Alternatively, sufficient residues of the antigen may be present so that secondary and tertiary structure is at least partially preserved, and antigenic regions are present that are not necessarily contiguous in primary sequence but are adjacent after folding of the molecule or generated by fusion of non-adjacent antigen sequences, as described for different ‘hybrid’ epitopes. The antigenic moiety of the construct may contain an epitope or multiple epitopes from one or from more than one antigen of interest. The multiple epitopes may be the same e.g. multiple copies of the same epitope may be present, or the epitopes may be different e.g. single or multiple copies of two or more different epitopes may be present. The epitopes may be “different” from (may differ from) each other either by virtue of originating from different antigenic molecules, or by originating from different parts of the same antigenic molecule, or both, e.g. the same region of an antigen from several different variants may be used. Combinations of the above may also be present. Post-translationally modified epitopes or epitopes generated by alternative splicing may equally be included. In addition, neo-epitopes can be generated by fusion of non-adjacent peptide stretches from the same Ag or even from different Ags.

The antigenic moiety may have a chemical composition that is the same as that of a naturally occurring antigen, e.g. if the antigen is a protein, polypeptide or peptide, its sequence may have 100% identity to that of a protein, polypeptide or peptide that is a natural antigen. Alternatively, the antigenic moiety may comprise a portion (e.g. at least about 10, 15, 20, 25, 30, 35, 40, 45, 50, 55, 60, 65, 70, 75, 80, 85, 90, 95% or more) of the residues of a native sequence to which immune tolerance is desired. Further, the amino acid sequence in the antigenic moiety may be the same primary sequence as that of a native protein antigen, or may be a variant thereof with at least about 50, 55, 60, 65, 70, 75, 80, 85, 90, 95% or more identity to the native protein sequence, or to the portion of the native sequence on which it is based. Similarly, the antigenic moiety may comprise a portion or substantially all (e.g. at least about 10, 15, 20, 25, 30, 35, 40, 45, 50, 55, 60, 65, 70, 75, 80, 85, 90, 95% or more) of the nucleotides of a native sequence of an entire nucleic acid molecule (generally dsDNA) to which immune tolerance is desired. The nucleic acid sequence in the antigenic moiety may be the same as that of the native sequence, or may be a variant thereof with at least about 50, 55, 60, 65, 70, 75, 80, 85, 90, 95% or more homology to the native sequence, or to a portion of the native sequence on which it is based.

The constructs may also provide various linker sequences and/or various detectable labels, as required or as desired in order to facilitate their production and administration. In addition, various other functionalities may be included in the constructs. Examples of such functionalities include, but are not limited to, domains of the antigenic protein that are required to exert one or several desired biological activities (e.g. binding to receptor(s) or avoidance of such binding) or that are modified so as to increase such biological activities. The constructs described herein may be produced by any of several known methods.

Typically, the constructs are recombinant proteins or polypeptides and are made by cloning and then translating nucleic acid sequences which encode all or a portion of the construct, using techniques that are familiar to those of skill in the art. Generally, the encoding sequence is present in a cloning or expression vector such as a genetically engineered plasmid, bacteriophage (such as phage A), cosmid, bacterial artificial chromosome (BAC), yeast artificial chromosome (YAC) or human artificial chromosomes (HAC). However, the constructs may also be produced synthetically e.g. by chemical synthesis.

The present invention provides compositions for use in eliciting tolerance to one or more antigens of interest. The compositions include one or more substantially purified constructs as described herein, or nucleic acid sequences encoding such constructs, and a pharmacologically suitable carrier. The preparation of such compositions for administration is well known to those of skill in the art. Typically, such compositions are prepared either as aqueous or oil-based liquid solutions, suspensions or emulsions, etc. However, solid forms such as tablets, pills, powders and the like are also contemplated, the solid forms being suitable for solution in, or suspension in, liquids prior to administration,. The active ingredients may be mixed with excipients which are pharmaceutically acceptable and compatible with the active ingredients, Suitable excipients are, for example, pharmaceutically acceptable salts, water, saline, dextrose, glycerol, ethanol and the like, or combinations thereof. In addition, the composition may contain minor amounts of auxiliary substances such as wetting or emulsifying agents, pH buffering agents, preservatives, and the like. The composition of the present invention may contain any such additional ingredients so as to provide the composition in a form suitable for administration. The final amount of construct in the formulations may vary. However, in general, the amount in the formulations will be from about 1-99%. Still other suitable formulations for use in the present invention can be found, for example in Remington's Pharmaceutical Sciences, Philadelphia, Pa., 19th ed. (1995). Some examples of materials which can serve as pharmaceutically acceptable carriers include, but are not limited to, ion exchangers, alumina, aluminum stearate, lecithin, serum proteins (such as human serum albumin), buffer substances (such as TWEEN® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, and antioxidants can also be present in the composition, according to the judgment of the formulator.

The constructs may be administered by any suitable route. For in utero delivery, typically, a composition comprising one or more types of construct is delivered to the infant indirectly in that delivery is to the mother, e.g. by injection (e.g. generally intravenous, or intraperitoneal). Any technique may be used, so long as the construct enters the circulatory system of the mother and is then transferred to the circulatory system of the fetus. In preferred embodiments, the mode of administration is intravenous.

In order to obtain the desired effect, e.g. complete or improved tolerance to at least one antigen of interest, and thus prevention or treatment of at least one symptom of a disease or condition caused by a lack of tolerance to the antigen, a therapeutically effective amount of the construct is administered. This amount is generally in the range of from about 10 μg to about 10 mg, and more preferably, is in the range of from about 100 μg to about 1000 μg (1 mg).

In some aspects, the constructs are administered during gestation and as soon as possible after pregnancy is confirmed. Thus, delivery is typically carried out after about 23 weeks of fetal development and usually at least by about 28 weeks of development. In other words, delivery is generally carried out between 28 and 40 weeks of gestation. However, in some cases, administration is carried out at any time up until birth. Further, one or multiple administrations (e.g. from about 2 to about 4 per week) may be undertaken.

The result of administration are generally monitored on an ongoing basis, e.g. by detecting or measuring the absence of alloimmunization to therapeutic FVIII during at least 100 cumulated exposure days or by detecting or measuring the absence of seroconversion for autoantibody markers of autoimmune diseases. By way of example, these autoantibodies are anti-insulin, anti-GAD, anti-IA-2 and anti-ZnT8 for type 1 diabetes. Any other suitable immunological readout may be used depending on the targeted condition, and these methods are known to the skilled in the art. The results of such tests may be compared to suitable controls, e.g. levels in a statistically significant group of healthy (negative) controls, levels in a statistically significant group of other subjects afflicted with the condition (positive controls), and/or levels in the fetus and/or mother prior to treatment, etc., to detect differences between the measured results and the control(s). For example, a decrease in the level of seroconversion compared to the level after treatment indicates that the treatment has been effective, as does detection of a level of seroconversion that does not differ statistically from healthy control levels, and/or a level of seroconversion that is lower than levels exhibited by positive control subjects. The results of such measurements are used to conclude whether or not to continue treatment, whether or not to modify the treatment (e.g. by increasing or decreasing the amount of active agent that is administered, or the frequency or total number of administrations, etc.).

Subjects who may benefit by the practice of the invention include any subject, usually a fetus, who is predisposed or believed to be predisposed to developing, or who has already developed or is developing, at least one symptom of a disease or condition caused by inappropriate or unwanted immune system activity against an antigen. The subject may be identified or diagnosed as having done so or as likely to do so based on a variety of factors, for example, family history and/or genetic testing of e.g. the mother and/or father, siblings, other relatives (grandparents, aunts, uncles, cousins, etc.); and/or based on other types of pre-natal assessment such as sampling of fetal blood or cells shed in amniotic fluid for the presence or absence of certain biomarkers. Generally, the subject is known to have a genetic predisposition to development of an autoimmune disease, condition and/or an allergy. By “is known to have a genetic predisposition” we mean that one or both parents or siblings have the disease or condition, and/or are known to be carriers of a gene that is associated with the disease or condition, so that the statistically probability of the fetus having or developing the disease is at least 1, 5, 10, 20, 30, 40, 50, 60, 70, 80, 90%, or is 100%. The determination may be based on observation of the health of the parents, or on genetic testing of the parents and identification of a gene or genes in a form known to be associated with or to cause the disease or condition, e.g. to have a particular sequence such as a mutation, insertion, deletion, etc. The risk of disease may or may not also be confirmed by genotypying fetal cells and/or by assessing them by suitable biomarkers. Those of skill in the art will also recognize that such genetic traits may not be “all or nothing”, in that gene dosage may apply. Nevertheless, if a subject is deemed to be at risk, and if the life of a subject can be lengthened or improved by the practice of the present methods, then the subject is a viable candidate for treatment.

The subjects who are treated as described herein may be, but are not necessarily, mammals. Frequently the subjects are human, but treatment of other animals is also encompassed, i.e. veterinary applications are included. For example, the offspring of companion pets and/or animals who are raised as livestock or for entertainment purposes may be treated, such as the offspring of prized breeding stock (horses, cattle, etc.). Further, cloned offspring may be treated in utero by these methods to ward of potential unwanted immune responses in the offspring after birth. Such offspring and the treatment of such offspring are also encompassed by the invention.

In some aspects, the disease that is prevented, treated or alleviated by practicing the invention is hemophilia and the antigen that is administered is FVIII, or a modified form thereof that retains the ability to elicit immune tolerance to at least one antigen that decreases or eliminates the symptoms of hemophilia in the subject who receives a construct as described herein. The FVIII may be human, porcine, canine, or murine in origin, or may be a chimera or hybrid of proteins or protein segments (e.g. domains) of different origins (e.g. human and porcine). In one embodiment, the B domain of Factor VIII is deleted (“B domain deleted factor VIII” or “BDD FVIII”). A “B domain deleted factor VIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. An exemplary B-domain deleted construct, FVIIIHSQ-F, is depicted in FIG. 19 (SEQ ID NO: 54). Modified or recombinant forms of FVIII may or may not function in coagulation (for instance the inactive FVIII(V) (634M) mutant as described in Gangadharan et al. 2014; reference 22 in References for Example 2 below). Modified forms of FVIII include, for example, those that do not bind to vWF, or for which the binding is weaker than or decreased compared to unmodified FVIII. Generally, the binding is at most 50% of the level of native FVIII, and is preferably 45, 40, 35, 30, 25, 20, 15, 10, or 5% of that of native FVIII, and binding may be altogether absent. Exemplary modifications of this type include FVIII in which amino acid residue 1680 is not tyrosine (or residue 1699 is not tyrosine, if the 19 amino acid leader sequence is included in the numbering of residues). For example, Tyr1680 may be mutated (e.g. by genetic engineering techniques) to create a recombinant FVIII in which position 1680 is Arg, His, Lys, Asp, Glu, Ser, Thr, Asn, Gln, Cys, Sel, Gly, Pro, Ala, Val, Ile, Leu, Met, Phe, and Trp, or a non-standard amino acid that interferes with the binding of vWF. Usually, Tyr1680 is substituted with Ala. Alternatively, one or more adjacent amino acids may be modified instead e.g. amino acids that are within from about 1-5 amino acids in primary sequence from position 1680, while Tyr at 1680 is left intact, but the molecule cannot bind or binds less well to vWF because of the changes. Alternatively, one or more residues that are not adjacent in primary sequence but which are brought into proximity to Tyr 1680, e.g. by the secondary or tertiary structure of FVIII, may be modified, e.g. to sterically occlude the area of FVIII that binds to vWF. FVIII molecules with any modification that decreases or eliminates the ability of FVIII to interact with and bind to vWF may be used in the preparation of the constructs described herein. In addition other modified versions of FVIII (e.g. with one or more amino acid substitutions, deletions, etc.) may also be employed, so long as the resulting polypeptide retains the ability to elicit useful immune tolerance to FVIII when administered as described herein. The amino acids sequence of native human (wild type) FVIII (SEQ ID NO: 50) is presented in FIG. 17A and the encoding nucleic acid sequence (SEQ ID NO: 51) is presented in FIG. 17B-E. Generally, the modified FVIII that is used is at least about 70, 75, 80, 85, 90, 95, 96, 97, 98 or 99% identical to the sequence presented in SEQ ID NO: 50, or to a segment of the sequence presented in SEQ ID NO: 50, i.e. if the FVIII is truncated or contains one or more internal deletions, the % identity refers to the remaining amino acids as they align within the sequence presented in SEQ ID NO: 50. Those of skill in the art will recognize that, in addition to SEQ ID NO: 51, many other nucleic acid sequences can encode SEQ ID NO: 50 due to redundancy in the genetic code, and all such sequences encoding modified forms of FVIII, especially when in a construct comprising an Fc component, are encompassed herein, including those in which position 1680 is not Tyr. Generally, such sequences will display at least about 70, 75, 80, 85, 90, 95, 96, 97, 98 or 99% homology to the sequence presented in SEQ ID NO: 51, or to a segment of the sequence presented in SEQ ID NO: 51, i.e. if the FVIII is truncated or contains one or more internal deletions, the % homology refers to the remaining nucleotides as they align within the sequence presented in SEQ ID NO: 51.

A number of modified factor VIII molecules which may be utilized or used as the basis of further modifications as described herein are disclosed in the following patents U.S. patents and applications: U.S. Pat. Nos. 6,316,226 and 6,346,513 (Van Ooyen et al.); U.S. Pat. No. 7,041,635 (Kim et al.); U.S. Pat. Nos. 6,060,447 and 6,228,620 (Chapman et al.); and 20150050278 (Dimitrov, et al.), each of which is incorporated herein by reference in its entirety.

As discussed herein, the constructs may be size modified or size optimized to insure or promote of facilitate transport across the placenta. Generally, the size of a construct will be less than about 320 kDa, e.g. about 300, 290, 280, 270, 260, 250, 240, 230, 220, 210, 200, 190, 180, 170, 160 or 150 kDa or less. Various methods of reducing the size of the constructs may be employed, such as those described in US patent applications 2002/0155537, 2007/0014794, and 2010/0254986 (each to Carter et al.), and 2014/0294821 (Dumont et al.), each of which is incorporated herein by reference in its entirety. For example, Fc-Fc and FVIII-Fc/FVIII-Fc dimer formation may be prevented, various domains of the antigen may be deleted (e.g. the B domain of FVIII may be deleted from FVIII-containing constructs); various domains of an Fc may be omitted from the construct e.g. only the CH2 domain may be used, or monomers comprising CH1, CH2 and CH3 may be utilized, among others.

Additional exemplary diseases and conditions, at least one symptom of which can be prevented, treated or alleviated by practicing the invention, are listed in Table 1. Representative antigens which are associated with the diseases and which may be targeted using the methods described herein are also presented.

TABLE 1

| Disease/Condition to be Treated | Exemplary antigen(s) for which tolerance is developed |
|---|---|
| systemic lupus erythematosus | the DWEYSVWLSN dsDNA-mimicking peptide (SEQ |
| | ID NO: 55) |
| | (Tchorbanov A, Eur J Immunol 2007, 37: 3587) |
| rheumatoid arthritis | various synovial antigens such as vimentin, nuclear |
| | ribonucleoprotein-A2 (RA33), fibrinogen, and |
| | alpha-enolase, and post-translationally modified forms |
| | thereof such as those generated by citrullination |
| type 1 diabetes | Insulin and its precursors proinsulin and preproinsulin |
| | (PPI), glutamic acid decarboxylase, |
| | insulinoma-associated antigen 2 (IA-2), zinc transporter |
| | 8 (ZnT8), islet-specific glucose-6-phosphatase catalytic |
| | subunit-related protein (IGRP), chromogranin A, islet |
| | amyloid polypeptide (IAPP) and its precursors, 78 kDa |
| | glucose-regulated protein (GRP78) and its precursors |
| | and other relevant beta-cell antigens described in the |
| | literature. |
| multiple sclerosis | myelin basic protein (MBP), myelin oligodendrocyte |
| | protein (MOG), proteolipid protein (PLP) |
| Addison's disease | 21-hydroxylase |
| autoimmune hepatitis | soluble liver antigen |
| autoimmune pancreatitis | lactoferrin, carbonic anhydrase |
| autoimmune thrombocytopenic | gpIIb-IIIa or 1b-IX |
| purpura | |
| celiac disease | tissue transglutaminase, gliadin |
| chronic inflammatory | GM1, GD1a, GQ1b |
| demyelinating polyneuropathy | |
| dermatomyositis | histidine-tRNA signal recognition peptide, Mi-2, Jo1 |
| pernicious anemia | intrinsic factor |
| mixed connective tissue disease | U1-RNP |
| myasthenia gravis | nicotinic acetylcholine receptor |
| narcolepsy | Orexin |
| pemphigus vulgaris | desmoglein 3 |
| polymyositis | IFN-γ, IL-1, TNF-α |
| primary biliary cirrhosis | p62, sp100, Ro |
| Rasmussen's encephalitis | NR2A |
| scleroderma | Scl-70, topoisomerase |
| Sjögren's syndrome | Ro, La |
| stiff man syndrome | glutamic acid decarboxylase (GAD) |
| autoimmune thrombocytopenia | glycoproteins IIb-IIIa, Ib-IX, ADAMTS13, cardiolipin, |
| | β 2-glycoprotein I, HPA-1a, HPA-5b |
| vitiligo | tyrosinase, Melan-A/MART-1, melanin-concentrating |
| | hormone receptor MCFI-R1, TRP-1, gp100, P protein |

Autoimmune syndromes comprising several autoimmune conditions may also be considered, including autoimmune polyendocrine syndromes, and immunodysregulation polyendocrinopathy enteropathy X-linked syndrome (IPEX); other immune-mediated disease including, but not limited to, allo-immunization following clotting factor replacement in hemophiliac, asthma, allergy and Pompe disease are also part of the conditions falling within the scope of this invention. These antigens include post-translationally modified and alternatively spliced isoforms and hybrid epitopes that may form by fusion of aminoacid sequences belonging to the same or to different antigens. Canonical antigens or antigen isoforms that are not properly expressed in the thymus may be particularly suitable to this end, including all antigens for all autoimmune diseases.

Other aspects of the invention encompass nucleic acid sequences (e.g. DNA, cDNA, RNA, mRNA, etc.) that encode the constructs described herein. In addition, cells comprising such nucleic acid sequences and/or comprising the constructs themselves are included. The cells may be, for example, host cells which have been genetically engineered to contain and express nucleic acids which encode the constructs, e.g. bacterial cells such as E.coli, B. subtilis, Pseudomonas fluorescens, gram-positive Corynebacteria, etc.), yeast (such as S.cerevisiae); or eukaryotic cell lines such as filamentous fungi, especially Aspergillus and Trichoderma; infected insect cells [13] (Sf9, Sf21, High Five strains); protozoan systems (e.g. Leishmania tarentolae); various plant systems (e.g. tobacco); mammalian systems or mammalian cells (e.g. HeLa, HEK 293, Bos primigenius, Mus musculus, Chinese Hamster Ovary (CHO) cells, Human Embryonic Kidney cells, Baby Hamster Kidney cells, etc.).

Those of skill in the art will recognized that, while complete elimination or eradication of all symptoms of a disease would be ideal, much benefit can still accrue even if the symptoms are only lessened so that the severity of disease is attenuated, or so that the symptoms are delayed so as to provide longer symptom-free period or symptom-decreased periods of time.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

### Example 1

### Regulation of Immune Responses to Antigens and Protein Therapeutics by Transplacental Induction of Central and Peripheral T-Cell Tolerance

In this Example, the physiological pathway by which maternal immunoglobulins are transferred to fetuses through the neonatal Fc receptor (FcRn) was exploited to non-invasively induce Ag-specific immune tolerance in the offspring. Using a hemagglutinin (HA)-specific T-cell receptor (TcR) transgenic mouse model, generation of HA-specific Tregs in the progeny was demonstrated following transplacental delivery of Fc-fused HA. This strategy was further translated to a preclinical model of severe hemophilia A, where congenital deficiency of pro-coagulant factor VIII (FVIII) leads to development of inhibitory anti-FVIII antibodies upon FVIII replacement therapy. Transplacental delivery of Fc-fused immunodominant FVIII domains was shown to provide long-lasting tolerance upon FVIII replacement therapy.

### Materials and Methods

### Mice and Cells

Homozygous mice that express a transgenic T-cell receptor (TCR-Tg) recognizing the HA₁₁₀₋₁₁₉ epitope (SFERFEIFPK, SEQ ID NO: 3) presented by I-E^{d}, on a Balb/c background, were bred in our animal facility. For experiments with pregnant mice, homozygous TCR-Tg males were crossed with wild-type Balb/c females purchased from Janvier Labs (St Berthevin, France). Eight to 11 week-old C56BL/6 WT and C56BL/6 FcRn−/− mice were obtained from Janvier (Saint-Berthevin, France) and The Jackson Laboratory (Bar Harbor, Me., USA), respectively. Factor VIII (FVIII)-deficient (HemA) mice were exon 16-knockout mice on a 129×C57B1/6 (H-2Db). Pregnant mice were obtained by homozygous crossing. Appearance of a vaginal plug was considered as day 0 of gestation. Pregnant mice were injected intravenously with 100 μg of Fc-fusion protein or mIgG1 (MOPC-21, BioLegend) on days 16, 17 and/or 18 of gestation. HA-TCR-Tg heterozygous offspring born from wild-type Balb/c females were used for analysis of Tregs at 2 weeks of age. The offspring from HemA females underwent replacement therapy with human recombinant FVIII (1 IU/mouse once a week for 4 weeks; HELIXATE® NexGen, CSL-Behring, Marburg, Germany) at 6 weeks of age. Plasma was kept at −20 ° C. until use. All animal experiments were performed in accordance with national animal care guidelines (EC directive 86/609/CEE, French decree no 87-848) with agreement from local ethical authorities: Comite Re_{g}ional d′Ethique p3/2008/024 and Comité Régional d'Éthique de Val-de-Loire 2012-04-9.

Mouse syncytiotrophoblast SC4235 cell line cells were cultured in Dulbecco's modified Eagle medium/Nutrient mixture F-12 with 10% fetal bovine serum (FBS) and 2 mM L-Alanyl-L-Glutamine, and grown in a 37° C., 5% CO₂ humidified incubator.

### Antigens

Peptide. The HA₁₁₀₋₁₁₉ peptide (SFERFEIFPK, SEQ ID NO: 3) was custom synthesized from Polypeptide Laboratories (Strasbourg, France) and was >97% pure as assessed by HPLC and mass spectrometry.

Fc-fusion proteins. Sequences encoding the A2 and C2 FVIII domains (pSP64-VIII, ATCC, Manassas, Va.), HA1 (pCI-Neo-HA, encoding HA) and Fcγ1 (B-cell hybridoma secreting mouse IgG1) were amplified by PCR employing specific oligonucleotides (Table 2). The sequences were digested with the appropriate restriction enzymes, purified and inserted at NheI/EcoRV sites by cohesive end ligation into pCDNA3.1(+) expression vector (INVITROGEN™). The expression was under the control of the CMV promoter and the expression cassette contains the signal peptide of IL-2, the c-myc sequence and the respective domain directly linked to the mouse Fcγ1. The different constructs were used to stably transfect HKB11 cells (ATCC) by electroporation. HKB 11 cells were grown in serum free HL1 medium (Lonza). All Fcγ1-fusion proteins were expressed in cell culture medium and purified by affinity chromatography, using agarose-coupled anti-mouse IgG (Sigma-Aldrich). Fractions were dialyzed against Phosphate buffer saline (PBS) and concentrated by ultrafiltration (AMICON® Ultra 30K device, Millipore). The chimeric proteins were validated by Western blot and ELISA, using domain-specific monoclonal antibodies.

TABLE 2
Oligonucleotide sequence of the primers used in cloning of Fcg1-fusion chimeric
proteins.

| ID | Primer Sequence (5′-3′) |
|---|---|
| Signal peptide ssIL-2 | NheIIL-2 s1: CTA GCT AGC ACC ATG TAC AGG ATG CAA CTC |
| | (SEQ ID NO: 4) |
| | IL2sacI as1: ATT GCA CTA AGT CTT GCA CTT GCT ACG AAC TCG |
| | GAG CTC GAG CAG AAA CTC ATC (SEQ ID NO: 5) |
| Tag c-myc | MyclinkbamHI s1: GC GAG CTC GAG CAG AAA CTC ATC TCT GAA |
| | GAG GAT CTG GGA TCC AGA TCT TC (SEQ ID NO: 6) |
| | MyclinkEcoRI s2: GC GAG CTC GAG CAG AAA CTC ATC TCT GAA |
| | GAG GAT CTG GAA TTC AGA TCT TC (SEQ ID NO: 7) |
| Domain HAI | HA1BamH1 s1: CGC GGA TCC GAC ACA ATA TGT ATA GGC |
| | (SEQ ID NO: 8) |
| | HA1BglII asl: GGA AGA TCT GGA TTG AAT GGA CGG AGT |
| | (SEQ ID NO: 9) |
| Domain A2 | A2EcoRI s1: GGA TTC TCA GTT GCC AAG AAG CAT |
| | (SEQ ID NO: 10) |
| | A2BglII as1: GCG AGA TCT TGG TTC AAT GGC ATT |
| | (SEQ ID NO: 11) |
| Domain C2 | C2BamHI s1: CG GGA TCC AAT AGT TGC AGC ATG CCA |
| | (SEQ ID NO: 12) |
| | C2BglII as1: GCC AGA TCT GTA GAG GTC CTG TGC CT |
| | (SEQ ID NO: 13) |
| Mouse IgG1Fc | IgG1mBglII s1: GCG AGA TCT GGT TGT AAG CCT TGC ATA TG |
| | (SEQ ID NO: 14) |
| | IgG1mEcorRV as1: CG GAT ATC GGA TCA TTT ACC AGG AGA GT |
| | (SEQ ID NO: 15) |
| T7 & BGH | T7: TAA TAC GAC TCA CTA TAG GG (SEQ ID NO: 16) |
| BGH reverse: | TAG AAG GCA CAG TCG AGG (SEQ ID NO: 17) |

Recombinant C2 protein. A C-terminal His tag was fused to the C2 domain of FVIII by cloning the coding sequence into the pET-22b(+) vector (Novagen). The C2 domain was produced into Escherichia coli Rosetta-gami7(DE3)pLysS upon induction by IPTG overnight at 15° C. Bacterial raw extract was obtained by sonication in lysis buffer (20 mM HEPES pH 7.2, 400 mM NaC1, 20 mM imidazole, 2.5% TRITON™ X-100, 1 mM PMSF, 0.8 mg/mL lysozyme). The C2 protein was purified by affinity chromatography (HisTrap HP, GE) with a linear gradient of 20 to 62.5 mM imidazole. C2 was further purified by size-exclusion chromatography on a Superose 6 10/300 GL (GE Healthcare), eluted by 20 mM HEPES pH 7.2, 150 mM NaCl, 2.5% glycerol. The C2-containing fractions were pooled and concentrated by ultrafiltration (AMICON® Ultra 10K device, Millipore).

### Immunofluorescence Microscopy

SC4235 cells, grown for one day on Ibidi chambers (Biovalley, Marne La Vallee, France), were washed with PBS and incubated for 10 min. in PBS at 37° C. The HA1Fc -ALEXA FLUOR® (AF) 647 was added at 100 μg/mL in PBS at pH 6 and the cells were incubated for 30 min at 37° C. Cells were rinsed in PBS, fixed in 4% paraformaldehyde (Electron microscopy sciences, Hatfield, PA) in PBS for 20 min at room temperature (RT) and permeabilized with 0.5% TRITON™-X100 for 20 min. The permeabilized cells were then stained for FcRn using anti-FcRn antibody (sc-46328, SantaCruz) followed by secondary antibody conjugated with FITC (sc-2024, SantaCruz) and nuclear staining (Hoechst 33342, Molecular Probes). Images were acquired using an Axiovert M200 microscope (Zeiss) equipped with Apotome and four filters (Dapi, FITC, Rhodamine, Cy5) connected to a monochromatique CCD camera. Digital images were captured with AxioVision software.

### Surface Plasmon Resonance Analysis

The kinetic constants of the interactions between mouse FcRn (mFcRn) and Fc-fusion proteins (HA1Fc, C2Fc, A2Fc) were determined using BlAcore 2000 (GE, Uppsala, Sweden). Biotinylated mFcRn was immobilized on sensor chip SA (GE Healthcare), as described by the manufacturer. In brief, mFcRn was diluted in tris/citrate buffer (100 mM Tris, 100 mM NaCl, 0.1% tween-20 and citric acid to adjust the pH at 5.4) to finally immobilize 1000 resonance units (RUs). Experiments were performed using tris/citrate buffer at pH 5.4, 6.4 or 7.4. Two-fold dilutions of Fc-fusion proteins or mIgG1 (from 200 to 0.78 nM) were injected at a rate of 30 μL/min. The association and dissociation phases were monitored for 5 min. The regeneration of the chip surface was performed by injecting tris/citrate buffer at pH 8.5 with a contact time of 30 sec. The binding to the surface of the control uncoated flow cell was subtracted from the binding to the mFcRn-coated flow cells. All measurements of the interaction of mFcRn with Fc-fusion proteins were performed at 25° C. BIAevaluation version 4.1 (Biacore) was used for the estimation of the kinetic rate constants. Calculations were performed by global analysis of the experimental data using the model of Langmuir binding with a drifting baseline included in the software.

### In Vitro Transcytosis Assay

SC4235 cells were grown onto 0.4 μm pore size transwell filter inserts (Corning Costar) to form a monolayer. The confluent monolayer was confirmed by staining with anti-ZO-1 antibody (INVITROGEN™). The HA1Fc protein or HAI alone were added at 5 μg/mL onto the apical chamber in 500 μl of RPMI-1640 medium supplemented with ultraglutamine (Lonza), 1% FBS, 1% non-essential amino acids, and 1% penicillin/streptomycin. The basolateral chamber was filled with the culture medium alone. The transcytosis of HA1Fc or HAI. was monitored from 1 to 24 hr at both 37° C. and 4° C. Supernatants from apical and basolateral chambers were collected at indicated time points and HA1Fc levels were determined by ELISA, using a rabbit polyclonal antibody to HAI (ab90602; Abcam) and a goat anti-mouse IgG-Horseradish Peroxidase (HRP) secondary antibody (SouthernBiotech). HAI. levels were determined by sandwich ELISA using the mouse monoclonal (ab128412, Abcam) and rabbit polyclonal anti-HA antibodies (ab90602, Abcam) followed by anti-rabbit IgG-HRP secondary antibody. The starting level of both the proteins in the culture medium loaded onto apical side was set as 100%. The levels of HA1Fc or HAI in apical and basolateral side were then estimated as relative to the percent of loaded protein.

### In Vivo Imaging

The Fc-fusion proteins were conjugated with AF680 using SAM Rapid Antibody AF680 labeling kit (INVITROGEN™), following the manufacturer's instructions. The pregnant WT C56B1/6 mice, FcRn−/− C56B1/6 mice, HemA mice and WT Balb/c mice were injected intravenously with 100 μg of either C2Fc-AF680 or HA1Fc-AF680, on day 18 of gestation (E18). Control pregnant mice were injected with PBS. Bio-luminescence imaging was performed with the IVIS-Lumina II imaging system (Perkin Elmer, Villebon-sur-Yvette, France). The mice were anesthetized and fluorescence images were obtained from the live animal 1 min, 4 hr and 24 hr after administration of the labeled proteins. Fetuses were dissected together with or without placenta 4 hr or 24 hr after injection, followed by analysis with the imaging system. Images were acquired with a Lumina II (Perkin Elmer) using dedicated filters (excitation: 675 nm; emission: 800±10 nm).

### Quantitative Analysis of Transplacental Antigen Transfer

To determine the role of the Fc-domain in transplacental transfer of chimeric HA1Fc, pregnant wild-type Balb/c mice were injected intravenously with 100 μg of HA1Fc or HA1 (11684-VO8H1, Sino Biological Inc.) on E19 of gestation. Blood and urine from pregnant mice were collected 5 min, 1 hr and 4 hr after injection. Fetuses were then removed and is blood was collected. Blood from 3 to 4 fetuses was pooled. Levels of HA1Fc or HA1 were determined in plasma and urine from pregnant mice and corresponding fetuses by ELISA. HA1 was detected using the mouse monoclonal and rabbit polyclonal anti-HA antibodies (ab128412, ab90602, Abcam) followed by anti-rabbit IgG-HRP secondary antibody (Thermo Scientific). The optical density obtained with the mothers' plasma 5 min after injection was set at 100%. The HA1Fc or HA1 levels were then estimated as a % relative to the starting levels in pregnant mice.

### Functional Characterization of HA1Fc

CD4⁻ T cells were isolated from the spleen of homozygous naïve HA-TCR-Tg mice using the Dynabeads® untouched mouse CD4 kit (INVITROGEN™). Cells were labeled with 5 μM CellTrace Violet (CTV cell proliferation kit, INVITROGEN™) for 15 min in PBS. Splenocytes from wild-type Balb/c mice depleted of CD4⁺ T cells were used as a source of antigen-presenting cells (APCs). The cells were co-cultured at 1 CD4⁺ T-cell: 2 APCs in U-bottom culture plates (Nunc) in complete proliferation medium (RPMI-1640 with ultraglutamine (Lonza) supplemented with 10 mM HEPES, 10% FBS, 1% non-essential amino acids, 1% sodium pyruvate, 50 μM 2-β-mercaptoethanol and 1% penicillin/streptomycin). The cells were then incubated with equimolar concentrations of HA1Fc, HA₁₁₀₋₁₁₉ peptide and mIgG1 (1.66 μM to 0.06 μM). After 5 days, the percent proliferation at different Ag concentrations was determined by gating on divided 6.5 TCR-Tg CD4⁺ T cells based on the CTV signal.

### Antibodies and Flow Cytometry Analysis

The following antibodies from BD Biosciences, e-Bioscience, BioLegend and R&D systems were used for the phenotypic analysis: peridininchlorophyll-protein (PercP)- or pacific blue (PB)-labeled anti-CD3, PB- or PercP-labeled anti-CD4, fluorescein isothiocyanate (FITC)- or PercP-labeled anti-CD8, allophycocyanin (APC)- or FITC-labeled anti-CD25, AF700- or APC-labeled anti-Foxp3, phycoerythrin (PE)-labeled TCR Vβ8.18.2, APC-labeled anti-Nrp-1 (Neuropilin-1), PB- or PE-labeled anti-CD11c, AF-700- or PE-labeled anti-CD11b, PB-labeled anti-CD45R, FITC-labeled anti-CD172a (SIRP-α), eFluor 450-labeled CD326 (EpCAM), AF-700-labeled anti-CD45, FITC-labeled anti-F4/80, PE-labeled anti-I-A^{d}/I-E^{d}, FITC-labeled anti-NK1.1 and PE-labeled anti-LY6G. The TCR-HA was identified using the PE-labeled anti-clonotypic 6.5 antibody. Unconjugated antibody to CD16/32 (2.4G2) was used to block Fc-receptors on cells. The AF-700- or APC-labeled anti-Foxp3 staining was performed using the eBioscience kit and protocol. Dead cells were excluded using fixable viability dye eFlour 506 (eBioscience). Isotype-matched irrelevant antibodies (BD Pharmingen) were used as controls. Acquisition was performed on a LSR II cytometer and data were analyzed using FlowJo (Tree Star) software.

### HA1Fc Uptake by Fetal Immune Cells

HA1Fc was conjugated with AF-647 using SAIVI AF-647 labeling kit (INVITROGEN™). Pregnant wild-type Balb/c mice were injected intravenously with 100 μg of HA1Fc-AF-647 on E19 of gestation. The fetuses were removed 24 hr later and the thymi, spleens and blood were collected. The tissues from 2 to 4 fetuses were pooled into one. Single-cell suspensions were obtained by enzymatic digestion in case of thymus and spleen, followed by filtration through 70 μm cell strainer (BD Falcon). Red blood cells (RBCs) were lysed using ACK lysis buffer (Lonza). The isolated cells were then stained with cell subset-specific antibodies in ice-cold buffer (1% FBS in PBS). Cells were defined as circulating dendritic cells (DCs) (CD11b⁺CD11c⁺SIRP-α⁺), thymic resident DCs (CD11b⁺CD11c⁺SIRP-α⁻), macrophages (CD11b⁺CD11c⁺F4/80⁺), B cells (CD11b⁻CD45R/B220⁺), splenic T cells (CD3⁺TCRVβ8.1/8.2⁺), thymic CD4+ single positive (SP) T cells (CD3⁺CD4⁺CD8⁻) and medullary thymic epithelial cells (CD45⁻CD11b⁻EpCAM⁺). HA1Fc-AF-647-positive cells were identified by gating on the live cells of each defined cellular subset. Percentages of HA1Fc-AF-647-positive cells were estimated among each subset.

### Splenocyte Proliferation Assay

Spleens were removed aseptically from 2-week-old mIgG1 or HA1Fc transplacentally treated heterozygous HA-TCR-Tg mice. Single splenocyte suspensions were prepared by mechanical dissociation, RBCs lysis and filtration through 70 μm cell strainers. Total splenocytes were stimulated with the HA₁₁₀₋₁₁₉ peptide (0 to 10 μg/mL) in complete proliferation medium. In the case of HemA mice, splenocytes were collected from 10-week-old mIgG1 or A2Fc+C2Fc transplacentally treated progeny, after replacement therapy with FVIII. Total splenocytes were stimulated with 0 to 10 μg/mL FVIII. Splenocytes from HemA mice were cultured in complete proliferation medium (supplemented with 2% FBS and 0.5% heat-inactivated serum from HemA mice). To define the proliferative capability of splenocytes from both treatment groups, the splenocytes were also stimulated with Concanavalin A (0 to 2 μg/mL, Sigma-Aldrich). After 48 or 72 hr of incubation, [³H]-thymidine (0.5 μCi/well) was added to the cell culture media for an additional 18 hr before harvest of cells. [³H]-thymidine incorporation was measured in a scintillation counter, and results of triplicates were expressed as mean counts per minute (cpm). The data are presented as proliferation index, calculated as the ratio of incorporated [³H]-thymidine in stimulated vs. non-stimulated cells.

### Assay for Anti-FVIII IgG

ELISA plates (MAXISORP™, Nunc) were coated with FVIII or recombinant C2 protein overnight at 4° C., and blocked with PBS-1% BSA for 1 hr at 37° C. Serum dilutions were then incubated for 1 hr at 37 ° C. Bound IgG was revealed using an HRP-conjugated anti-mouse IgG (SouthernBiotech) and the substrate o-Phenylenediamine dihydrochloride (Sigma-Aldrich). The mouse monoclonal anti-FVIII IgG mAb6 (a gift from Prof J. M. Saint-Remy, KUL, Belgium) or ESH8 (American Diagnostica Inc., Stamford, CT) were used as standards. For this reason, levels of anti-FVIII IgG are arbitrary and are expressed as mg/mL mAb6 or ESH8 equivalent.

### Titration of FVIII Inhibitors

Heat-inactivated plasma was incubated with a standard pool of human plasma (Siemens, Saint-Denis, France) for 2 hr at 37 ° C. The residual pro-coagulant FVIII activity was measured using the TVIII chromogenic assay kit' following the manufacturers recommendations (Siemens). One Bethesda unit expressed in BU/mL is defined as the reciprocal of the dilution of plasma that produces 50% residual FVIII activity.

### Treg Suppression Assay

Spleens were dissected out from mIgG1 or A2Fc+C2Fc transplacentally treated HemA progeny. The spleens from 4-6 mice in each treatment group were pooled and mechanically dissociated. Splenic CD4⁺CD25⁺ Treg cells were isolated by magnetic selection, using mouse regulatory T-cell isolation kit (Miltenyi Biotec). For FVIII-specific suppression, untouched CD4⁺CD25⁻ responder T cells were isolated from HemA mice challenged with FVIII (5 IU/mouse, once a week for 4 consecutive weeks), following the kit protocol (DYNABEADS® untouched mouse CD4, INVITROGEN™). Responder CD4⁺CD25⁻ T cells were labeled with CTV-proliferation dye, as described above. Splenocytes from FVIII-challenged mice depleted of CD4⁻ T cells, were treated with mitomycin (Sigma-Aldrich) and used as APCs. The CD4⁺CD25⁺ Tregs were co-cultured with CTV-labeled responder CD4⁺CD25⁻T cells (Teffs) at 1:2 and 1:1 Tregs: Teffs ratios, with similar numbers of APCs. The co-cultured cells were stimulated with FVIII at 1 μg/mL in complete proliferation medium. After 72 hr, percentage proliferation was determined by gating on CD4⁺ T cells based on CTV dilution. The percent proliferation of Teffs in the absence of CD4⁺CD25⁺ Tregs was set as 100%. The relative suppression in proliferation of Teffs in the presence of Tregs from both the treatment groups was estimated as percent suppression.

### Adoptive Transfer of Tregs

Spleens were removed from 3-week-old mIgG1 or A2Fc+C2Fc transplacentally treated HemA progeny. Spleens were pooled from each treatment group and CD4⁺CD25⁺ Tregs were isolated as described above. A total of 1×10⁶ cells suspended in 200 μL of PBS was injected into the tail vein of naïe 6-week-old HemA recipients. As an additional control, naïve HemA recipients were injected with PBS. Twenty-four hours after adoptive transfer, all the groups experienced replacement therapy with FVIII (1 IU/mouse/week) for 4 weeks. Plasma collected one week after the last FVIII injection was analyzed for anti-FVIII IgG titer by ELISA, as described above.

### Statistical Analysis

In all experiments, data are expressed as means±SEM. The statistical significance of differences between groups were evaluated using the two-tailed student t-test, two-sided Mann-Whitney U test or by two-way ANOVA with Bonferroni post-hoc test when indicated. Statistical analyses were performed using the GraphPad Prism 5.0b software (GraphPad Software, San Diego, Calif., USA).

### Results

### HA1Fc Binds to Neonatal Fc Receptor and is Transcytosed by Syncytiotrophoblast Cells

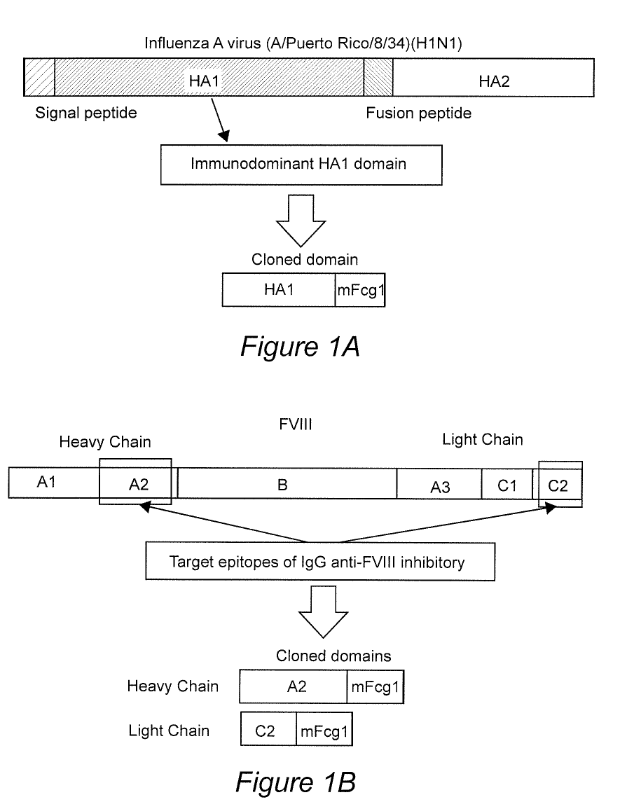

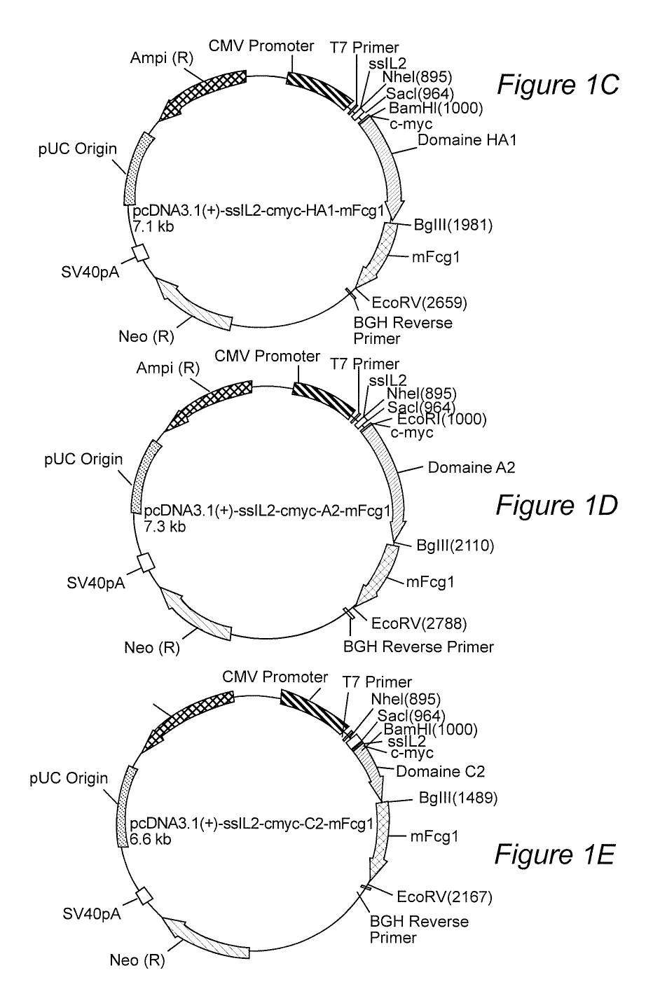

The neonatal Fc receptor (FcRn) is crucial for Fcγ-dependent transcytosis of maternal IgG across placenta. As a model Ag for transplacental studies, we produced an Fcγ1-coupled HA1 (HA1Fc) (FIG. 1A-E). Nuclear staining with Heochst 33248 (not shown) showed that HA1Fc co-localized with FcRn in syncytiotrophoblast cells, suggesting its interaction with FcRn. Surface plasmon resonance analysis of real-time interaction profiles of the binding of increasing concentration (0.39 to 200 nM, two-fold dilutions) of mIgG1 and HA1Fc to immobilized mouse FcRn, at varying pH, showed that HA1Fc and a mouse monoclonal IgG1 (mIgG1) displayed similar binding affinities, and similar pH-dependency in the interaction with FcRn. The results indicate that the affinity of the Fcγ1 for FcRn is not influenced by the fused protein. In vitro transcytosis through syncytiotrophoblast cells of HA1Fc was revealed in a transwell assay. In the assay, the cell monolayer on the transwell filter was apically exposed to HA1Fc (2.5 μg) and levels of HA1Fc in the supernatant from apical (upper well) and basolaterial (lower well) were determined by ELISA. The results showed a time-dependent increase in basolateral levels with a decrease in apical levels (not shown). Absence of transcytosis of HA1-Fc at 4° C. together with absence of transcytosis of HA1 alone at 37° C. suggested an active and Fc-dependent transfer of HA1Fc. Altogether, HA1Fc has the biochemical characteristics required for efficient transplacental transfer. Materno-fetal transfer of HA1Fc is Fc-dependent

In vivo imaging of pregnant wild-type (WT) or FcRn knock-out (FcRn−/−) mice injected intravenously with HA1Fc at embryonic day 18 (E18), revealed HA1Fc accumulation in the liver of both the strains at 1 min (FIG. 2A-B, top panels). The transplacentally transferred HA1Fc was detectable in the placenta of fetuses from both WT and FcRn−/− mothers (FIG. 2A-B, bottom panels), but was detectable only in WT fetuses both 4 and 24 hr after injection to mothers, while it was not detected in fetuses from FcRn−/− mothers (FIG. 2C-D). The analysis of blood collected from fetuses of both strains further validated the absence of HA1Fc transfer to FcRn−/− fetuses (FIG. 3A and B). By 24 hr, HA1Fc was also detected in the thymus of WT fetuses (not shown). In order to confirm the role of the Fc domain in the transplacental transfer of HA1Fc, we followed the levels of HA1Fc and of HA1 alone in mothers and fetuses following intravenous injection to pregnant mice. HA1Fc levels increased in a time-dependent manner in fetal plasma to reach 44±5% of the injected protein by 4 hr (FIG. 2E), with a simultaneous decrease from 100 to 22±2% in mothers' plasma. In contrast to HA1Fc, Fc-devoid HA1 was not detected in fetal plasma, despite a time-dependent and swift decline in mothers' plasma (down to 6±1% in 4 hr) (FIG. 2F). Taken together, these data suggest that approximately one-third of the HA1Fc is transferred to fetuses within 4 hr following administration to mothers, and that the transplacental transfer is Fc-dependent.

In order to validate the integrity of HA1Fc as a target Ag for immune effectors, we evaluated the efficacy of HA1Fc in inducing the proliferation of HAI-specific TCR-Tg CD4⁺ T cells (FIG. 2G). At 60 nM, HA1Fc induced a 3-fold greater proliferation of TCR-Tg CD4⁺ T cells than the HA₁₁₀₋₁₁₉ peptide (70.1±4.3% versus 19.4±0.6%), suggesting better presentation by Ag-presenting cells (APCs).

### Transplacental Delivery of HA1Fc Induces Tregs in an Ag-Specific Manner

We then addressed the potential of HA1Fc to shape the fetal immune repertoire. Based on the developmental phases of the mouse immune system, on the half-life of HA1Fc (approximately 6 hr) and given that thymic TCR expression is first detected around E17, we administered HA1Fc at different frequencies over the E16 to E18 gestational window. The optimal delivery schedule to significantly modulate HA1-specific Tregs (Tg-Tregs) was determined by analyzing Tg-Tregs in spleens of transplacentally treated mice 2 weeks after birth (1-3). Transplacental delivery to fetuses of 100 μg HA1Fc daily on E16, E17 and E18 optimally induced Tg-Tregs in neonates, as compared to delivery on E16 and E18, or E16 only (not shown). A thorough analysis of the modulation of different T-cell subsets upon HA1Fc transfer on E16-E17-E18 was then conducted. Frequencies of TCR-Tg (6.5⁺, recognized by the anti-clonotypic antibody 6.5) or non-Tg (6.5⁻) total CD4⁺ T cells were similar in the spleens of mice treated transplacentally with mIgG1 or HA1Fc (FIG. 4A). However, the frequency of Tg-Tregs in spleen was significantly increased by 2.6 folds in HA1Fc-treated mice as compared to mIgG1-treated mice (FIG. 4B, top panel and FIG. 4A, right panel). Furthermore, we observed a marginal yet significant decrease in the frequency of transgenic effector T cells (Tg-Teff) following HA1Fc treatment as compared to mIgG1 treatment (FIG. 4B, middle panel). The fact that deletion of Tg-Teff was more prominent when HA1Fc was transplacentally delivered on E16 and E18 (not shown), may reflect differences in thymic selection thresholds compared to Tregs.

The frequency of Tg-Tregs also showed a more than 6-fold significant increase in the thymus of HA1Fc-treated mice as compared to mIgG1-treated mice (FIG. 4C), which reflects the fact that tolerance is initiated in thymus between E16-E18. The few number of cells in the thymus at the time of analysis probably results from egress of these cells to the periphery. The frequency of Tg-Teff in the thymus remained unaltered irrespective of HA1Fc or mIgG treatment (FIG. 4C). HA-TCR-Tg mice express only 10-15% of TCR Tg CD4⁺ T cells (FIG. 4A), allowing analysis of the effect of HA1Fc transfer on the non-specific T-cell subsets. There was no significant modulation of non-transgenic Treg or Teff subsets in both spleen (FIG. 4B) and thymus (FIG. 4C).

CD8⁺ single positive (SP) cells in these mice also express a Tg-TCR. However, the frequency of Tg and non-Tg CD8⁺ T cells was affected neither in spleen nor in thymus (FIGS. 4B and C, bottom panels), thus confirming that the observed Ag-specific effects were MHC class II-restricted.

Tregs may be categorized into thymic-derived or natural (nTreg), and peripherally or adaptively induced (iTreg) subsets, based on the expression of Nrp-1 (Neuropilin-1). Moreover, the induction and expansion of iTregs by the administration of foreign Ags has already been shown. We found a two-fold significant increase in the frequency of Tg-nTregs in the spleen of HA1Fc-treated mice as compared to mIgG1-treated mice (FIG. 4D). Strikingly, the frequency of Tg-iTregs showed a significant 4.5-fold increase in the case of transplacental delivery of HA1Fc over mIgG1 (FIG. 4D). In the case of non-Tg Tregs, there was no modulation observed in both nTreg and iTreg subsets (FIG. 4D). We found significantly higher numbers of Tg-nTregs in the thymus of HA1Fc- (4.7-fold increase) vs. mIgG1-treated mice (FIG. 4E). As expected, Tg-iTregs were not detected in the thymi from mice of both the groups. Similar to spleen, non-Tg nTregs and iTregs remained unaltered in the thymus (FIG. 4E). Altogether, these data highlight an overall increase in HA1Fc-specific Tregs for both nTregs and iTregs. This increased number of both Treg subsets may be due to the relatively high affinity of the 6.5 TCR for the HAI peptide/MHC complex and non-inflammatory Ag encounter in the periphery as well as in the thymus. The precise mechanism underlying this phenomenon remains, however, to be investigated.

In the presence of their cognate Ag, Tregs typically exert potent suppression of Teff proliferation. We thus evaluated the suppressive potential of Tregs generated upon transplacental transfer of HA1Fc. Upon stimulation with the HA₁₁₀₋₁₁₉ peptide, splenocytes from HA1Fc-treated mice showed a more than two-fold reduction in proliferation as compared to splenocytes from mIgGl-treated mice (p<0.001, FIG. 4F, left panel). Conversely, splenocytes from HA1Fc-treated and mIgG1-treated mice proliferated equally upon concanavalin A stimulation, thus excluding splenocyte anergy (FIG. 4F, right panel). Thus, transplacental HA1Fc delivery generates Tregs in an Ag-specific manner that seem to be functional in suppressing Teff in the presence of their cognate Ag.

### Transplacentally-Delivered HA1Fc is Endocytosed by Fetal APCs of Myeloid Origin

Next, the nature of the APCs that contribute to the central and peripheral selection of

Tregs upon transplacental Ag delivery was investigated. To this end, we identified the APC subsets that endocytose maternally delivered ALEXA FLUOR® 647-labeled HA1Fc. SIRP-α⁺ circulating dendritic cells (DCs) were the major cellular subset that endocytosed transplacentally delivered HA1Fc: HA1Fc was detected in 20, 11 and 6% of SIRP-α⁺ cells in the fetal thymus, spleen and blood, respectively (FIG. 5). SIRP-α⁺ DCs are characterized as a migratory subset capable of ferrying blood-borne Ags to the thymus, thus leading to immune tolerance induction by negative selection of Ag-specific Teffs and positive selection of nTregs. HA1Fc was not detected in SIRP-α⁻ thymic resident DCs or SIRP-α⁻ DCs in the spleen (FIG. 5), while SIRP-α⁻ DCs were absent in blood. Transplacentally delivered HA1Fc was also present in fetal thymic, spleen and blood macrophages (10, 6 and 4%, respectively), but neither in B and T cells (FIG. 5), nor in medullary thymic epithelial cells (data not shown). These data implicate fetal APCs of myeloid origin—mostly SIRP-α⁺ migratory DCs - in the induction of Tregs specific for the administered HA1Fc. Transplacentally-transferred Fe-fused FVIII domains induce tolerance to therapeutic FVIII in experimental hemophilia A

Finally, we exploited this strategy to impose tolerance towards self Ags not expressed in genetic deficiencies. We thus translated our approach in FVIII-deficient (HemA) mice, a model of severe hemophilia A which develop inhibitory antibodies to therapeutic FVIII upon replacement therapy, as in patients. The A2 and C2 domains of FVIII are the major immunogenic determinants Therefore, we constructed two chimeric A2Fc and C2Fc proteins (FIG. 1B-E), endowed with affinities and acid pH-dependency for binding to FcRn equivalent to that of mIgG1 (FIG. 6A). In vivo imaging following administration of C2Fc-ALEXA FLUOR® 680 to pregnant mice on E18 illustrates accumulation of C2Fc in the placenta of both WT and FcRn−/− fetuses (FIG. 6B, top and bottom panel). However, the transplacental transfer to fetal circulation was only observed in fetuses from WT mice by 4 hr, as well as by 24 hr, and not in fetuses from FcRn−/− mice (FIG. 6, C-D) and ELISA (FIGS. 6, E-F and FIG. 3B). As observed for HA1Fc, transplacentally transferred C2Fc was detected in thymus of WT fetuses by 24 hr (not shown).

The progeny of mothers injected with A2Fc and/or C2Fc or with mIgG1 at E16-E17-E18 subsequently received replacement FVIII therapy from 6 weeks of age onwards (FIG. 7A). As a model antigen, the immune response to C2 domain of FVIII was analyzed in the progeny of mothers injected with C2Fc or with mIgG1. The Anti-C2 IgG titers were negligible in C2Fc (mean±SEM: 0.008±0.01 mg/mL ESH8-equivalent, p=0.0003) transplacental treatment groups, as compared to mIgG1 treatment (0.08±0.01 mg/mL FIG. 7B). Moreover, transplacental delivery of both A2Fc/C2Fc led to a remarkable reduction in total anti-FVIII IgG titers (FIG. 7C, expressed as mg/mL mAb6-equivalent): 1.5±0.5 mg/mL for A2Fc alone (p=0.07), 0.9±0.2 mg/mL for C2Fc alone (p=0.0006) and 0.4±0.2 mg/mL for A2Fc+C2Fc (p=0.002), as compared to mIgG1 (3.0±0.6 mg/mL).

Importantly, inhibitory titers were drastically reduced in the A2Fc+C2Fc treatment group (81±31 BU) as compared to the mIgG1 treatment group (591±155 BU, p=0.004, FIG. 7D).

Because a role for FVIII-specific Tregs has been evoked in the establishment of tolerance to therapeutic FVIII and because transplacental delivery of HA1Fc generates HA-specific Tregs, we investigated the induction of FVIII-specific Tregs upon transplacental treatment with A2Fc+C2Fc. In the presence of FVIII, splenocytes from the offspring of A2Fc+C2Fc-treated mothers proliferated to a lesser extent (proliferation index between 2 and 3) than that of progeny from mIgG1-treated mice (proliferation index between 3.7 and 6, p<0.05, FIG. 8A, left panel). The proliferative capacity of the splenocytes to mitogen stimulation was however unaltered (FIG. 8A, right panel). Altogether, these results suggest the induction of Tregs upon A2Fc+C2Fc transplacental delivery.

An inherent limitation of the HemA mice, that mount polyclonal responses to FVIII, is that phenotypic identification of FVIII-specific Tregs is not feasible. We therefore relied on the functional measurement of the suppressive activity of Tregs isolated from animals subjected to different treatments. In an in vitro assay, in the presence of FVIII, Tregs from the spleen of mice treated transplacentally with A2Fc+C2Fc significantly reduced the proliferation of CD4⁺CD25⁻ Teff from FVIII-primed mice, as compared to Tregs from mIgG1-treated mice (FIG. 8B). Furthermore, the adoptive transfer of Tregs from A2Fc+C2Fc transplacentally treated mice into naive HemA mice significantly reduced the antibody response against FVIII upon replacement therapy, as compared to Tregs from mIgG1 -treated mice (p=0.004) (FIG. 8C). Altogether, the diminished antibody response against FVIII is attributable to FVIII-specific Tregs generated upon transplacental treatment with FVIII domain-Fc fusion proteins.

### Discussion

The present work provides a novel in utero therapeutic strategy to manipulate the T-cell selection process during immune ontogeny and to induce Ag-specific immune tolerance. Here, we show that administration to pregnant mice of Fc-fused Ags results in effective Ag transfer to the fetal circulation in an FcRn-dependent pathway. Importantly, we demonstrate that the transplacental transfer of Fc-fused Ags induces an increase of thymic and peripherally derived Tregs in an Ag-specific manner. Fc-fused Ags are taken up by fetal APCs of myeloid origin both in thymus and periphery, suggesting a role for these cells in the establishment of central and peripheral tolerance. When translated to a preclinical model of severe hemophilia A, transplacental Ag delivery induced tolerance towards therapeutic FVIII in the progeny.

The immunogenicity of protein therapeutics is a major obstacle for the management of several conditions, as development of immune responses following their administration in patients neutralizes therapeutic benefit. The unwanted immunogenicity of biological drugs is linked to both extrinsic factors, such as processes associated with manufacturing, and intrinsic factors, such as recognition of few epitopes as foreign by the immune system of the patients. The latter is particularly relevant for replacement therapies in patients with genetic deficiencies, where the whole biologic agent may be recognized as foreign. In this context, T cells are central in the immune responses to these protein drugs, and therefore, strategies modulating the T-cell repertoire towards immune tolerance may provide means to avoid such responses.

Our study provides a strategy that modulates the T-cell repertoire in an Ag-specific manner and generates Tregs that are crucial to achieve immune tolerance. The approach was successfully translated into a mouse model of severe hemophilia A, a disease that may benefit the most from the induction of matemo-fetal tolerance. Our findings should be translatable to the human situation. Indeed, while obvious differences exist between mice and human in terms of length of gestation, maturity of the immune system and life span, both organisms share a similar time-frame for immune-intervention during gestation. In the human, FcRn-mediated transplacental transfer of mother IgG initiates at week 16 of pregnancy and increases thereafter; it was reported to be more efficient than in the rodent (14). Moreover, T-cell development and thymic colonization occur in the first trimester of human fetal development and the first mature thymocytes in the human fetus are detectable as early as week 12 to 14 of pregnancy. This time-frame corresponds to the first detection of thymic TCR expression and to the ontogeny of T cells in the mice during E16-E18 (17-19). These facts indicate that, like in mice, there is a favorable time window for shaping central T-cell tolerance during fetal development in the human. In the context of hemophilia A, the birth of hemophilic boys may be anticipated based on a family history of hemophilia and on prenatal screening. Furthermore, prenatal genetic diagnosis may also predict patients with the highest risk of developing anti-FVIII antibodies upon replacement therapy. It is thus possible to identify patients that would benefit the most from the induction of materno-fetal tolerance. Importantly, prenatal genetic diagnosis is possible from the 12^{th} week of pregnancy, offering the possibility of in utero immuno-intervention.

In patients with severe hemophilia A, the first bleeding episodes generally occur at the time of delivery or within the first 14 months of life. Moreover, inhibitory anti-FVIII antibodies develop during the first 50 cumulative days of exposure to therapeutic FVIII. It is plausible that materno-fetal delivery of Fc-coupled FVIII domains to hemophilia A patients may provide tolerance to FVIII lasting long enough to cover the most critical period for development of FVIII inhibitors. Interestingly, the use of prophylaxis in patients with hemophilia A, which consists in administration of FVIII every 2 to 3 days, sets the stage for the maintenance or rapid turnover of the FVIII-specific Tregs that would have been induced by our strategy.

The potential of this approach is underscored by the fact that the sole use of immunodominant A2 and C2 domains of FVIII, which together cover only 20% of the whole FVIII sequence, was sufficient to reduce immune responses in hemophilic mice by more than 80%. A further proof of concept is provided by the fact that treatment of pregnant mice with the Fc-fused C2-domain completely abrogated the immune response to the C2 domain of FVIII in the offspring. Alternatively, or in addition, combining the use of A2Fc/C2Fc in utero with adjunct strategies for tolerance after birth, such as oral delivery of bio-encapsulated FVIII domains, could be envisaged.

The present work also paves the way towards translation to autoimmune disorders for which the target antigens have been identified, such as autoimmune type 1 diabetes and to other genetic deficiencies, such as diabetes (see Example 2), hemophilia B (for which a therapeutic factor IX-Fc has recently been validated), or infantile Pompe disease, all of which become life-threatening upon the occurrence of neutralizing antibodies following replacement therapy.

### REFERENCES FOR BACKGROUND AND EXAMPLE 1

1. S. Dasgupta, J. Bayry, S. Andre, J. D. Dimitrov, S. V. Kaveri, S. Lacroix-Desmazes, Auditing protein therapeutics management by professional APCs: toward prevention of immune responses against therapeutic proteins. J. Immunol 181, 1609-1615 (2008).
2. S. D. Miller, D. M. Turley, J. R. Podojil, Antigen-specific tolerance strategies for the prevention and treatment of autoimmune disease. Nat. Rev. Immunol 7, 665-677 (2007).
3. K. E. Webster, S. Walters, R. E. Kohler, T. Mrkvan, O. Boyman, C. D. Surh, S. T. Grey, J. Sprent, In vivo expansion of T reg cells with IL-2-mAb complexes: induction of resistance to EAE and long-term acceptance of islet allografts without immunosuppression. J. Exp. Med 206, 751-760 (2009).
4. C. Daniel, K. Wennhold, H. J. Kim, H. von Boehmer, Enhancement of antigen-specific Treg vaccination in vivo. Proc. Natl. Acad. Sci. U.S.A 107, 16246-16251 (2010).
5. S. Sauer, L. Bruno, A. Hertweck, D. Finlay, M. Leleu, M. Spivakov, Z. A. Knight, B. S. Cobb, D. Cantrell, E. O'Connor, K. M. Shokat, A. G. Fisher, M. Merkenschlager, T cell receptor signaling controls Foxp3 expression via PI3K, Akt, and mTOR. Proc. Natl. Acad. Sci. U.S.A. 105, 7797-7802 (2008).
6. G. Liu, S. Bums, G. Huang, K. Boyd, R. L. Proia, R. A. Flavell, H. Chi, The receptor S1P1 overrides regulatory T cell-mediated immune suppression through Akt-mTOR. Nat. Immunol 10, 769-777 (2009).
7. G. Marodon, S. Fisson, B. Levacher, M. Fabre, B. L. Salomon, D. Klatzmann, Induction of antigen-specific tolerance by intrathymic injection of lentiviral vectors. Blood 108, 2972-2978 (2006).
8. M. S. Jordan, A. Boesteanu, A. J. Reed, A. L. Petrone, A. E. Holenbeck, M. A. Lerman, A. Naji, A. J. Caton, Thymic selection of CD4+CD25+ regulatory T cells induced by an agonist self-peptide. Nat. Immunol 2, 301-306 (2001).
9. I. Apostolou, A. Sarukhan, L. Klein, H. von Boehmer, Origin of regulatory T cells with known specificity for antigen. Nat. Immunol 3, 756-763 (2002).
10. M. Guerau-de-Arellano, M. Martinic, C. Benoist, D. Mathis, Neonatal tolerance revisited: a perinatal window for Aire control of autoimmunity. J. Exp. Med 206, 1245-1252 (2009).
11. R. E. Billingham, L. Brent, P. B. Medawar, Actively acquired tolerance of foreign cells. Nature 172, 603-606 (1953).
12. J. D. Fontenot, J. L. Dooley, A. G. Farr, A. Y. Rudensky, Developmental regulation of Foxp3 expression during ontogeny. J. Exp. Med 202, 901-906 (2005).
13. S. P. Berzins, R. L. Boyd, J. F. Miller, The role of the thymus and recent thymic migrants in the maintenance of the adult peripheral lymphocyte pool. J. Exp. Med 187, 1839-1848 (1998).
14. M. Asano, M. Toda, N. Sakaguchi, S. Sakaguchi, Autoimmune disease as a consequence of developmental abnormality of a T cell subpopulation. J. Exp. Med 184, 387-396 (1996).

### EXAMPLE 2

### Materno-Fetal Transfer of Preproinsulin (PPI) Through the Neonatal Fc Receptor Prevents Autoimmune Diabetes

The first signs of autoimmune activation leading to β-cell destruction in type 1 diabetes (T1D) appear during the first months of life. The corollary to these observations is that prevention strategies should be implemented much earlier, in children carrying a high HLA-associated genetic risk but with no sign of active autoimmunity (i.e. auto-Ab⁻) (1). The perinatal period offers such opportunities not only in terms of timing, but also because it is characterized by immune responses to introduced Ags that are biased towards tolerogenic outcomes. Indeed, Ag introduction during fetal life results in Ag-specific immune tolerance persisting during adulthood (2, 3). A key role in this process is played by central tolerance, since thymic negative selection of autoreactive effector T-cells (Teffs) and positive selection of regulatory T-cells (Tregs) is very active during this period and defines the immunological self that later imprints peripheral immune responses (4).

Thus, the perinatal period offers a suitable time window for disease prevention. Moreover, thymic selection of autoreactive T-cells is most active during this period, providing a therapeutic opportunity not exploited to date. We therefore devised a strategy by which the T1D triggering antigen preproinsulin fused with the immunoglobulin (Ig)G Fc fragment (PPI-Fc) was delivered to fetuses through the neonatal Fc receptor (FcRn) pathway, which physiologically transfers maternal IgGs through the placenta. PPI-Fc administered to pregnant PPI_{B15-23} T-cell receptor-transgenic mice efficiently accumulated in fetuses through the placental FcRn and protected them from subsequent diabetes development. Protection relied on ferrying of PPI-Fc to the thymus by migratory dendritic cells and resulted in a rise in thymic-derived CD4⁺ regulatory T-cells expressing transforming growth factor β and in increased effector CD8⁺ T-cells displaying impaired cytotoxicity. Moreover, polyclonal splenocytes from non-obese diabetic (NOD) mice transplacentally treated with PPI-Fc were less diabetogenic upon transfer into NOD.scid recipients. Transplacental antigen vaccination provides a novel strategy for early T1D prevention, further applicable to other immune-mediated conditions.

### Research Design and Methods

### Generation of PPI1-Fc and PPI2-Fc Fusion Proteins

Sequences encoding PPI1 and PPI2 were PCR-amplified from pancreatic and thymic cDNA, respectively, obtained from an 8-wk-old non-diabetic NOD mouse (see Table 3 and FIG. 9A-C for primer sequences, cloning, expression and purification strategies). The anti-CD20 rituximab monoclonal Ab (mAb; Roche) was used as IgG1 control. See also FIGS. 18A and B for the nucleotide and amino acid sequences of PPI1, SEQ ID NOS: 52 and 53, respectively.

Sequences encoding PPI1 and PPI2 were PCR-amplified from pancreatic and thymic cDNA, respectively, and inserted into pCR4-TOPO plasmids (Invitrogen). Following digestion with the appropriate restriction enzymes, PPI1/2 sequences were inserted at EcoRV/BglII sites by cohesive end ligation into pFUSE-hIgG1-Fc2 expression vector (InvivoGen), downstream of an IL-2 signal peptide and upstream of the human Fc γ 1 sequence. PPI1-Fc and PPI2-Fc sequences were then re-amplified by PCR and ligated at XbaI/XhoI sites into the pFastBac1 expression vector (Invitrogen). These constructs were inserted into the Bac-to-Bac Baculovirus Expression System (Invitrogen), expressed in Hi5 insect cells and protein products purified on Sepharose-coupled protein G (GE Healthcare). Protein identity was confirmed by reducing SDS-PAGE and Western blot using rabbit anti-insulin polyclonal Ab (H-86, Santa Cruz) and mouse anti-human Fc mAb (Southern Biotech). PPI1 and PPI2 were purified from Hi5 insect cell pellets as previously described (7)

TABLE 3
Oligonucleotide sequences of primers used for cloning and expression of
PPI-Fc and PPI constructs.

| Construct | Target plasmid | Primer Sequences |
|---|---|---|
| PPI1 | pCR4-TOPO | 5′ ATG GCC CTG TTG GTG CAC TTC (SEQ ID NO: 18) |
| | | 3′ AGA TCT ACC GCC GCC ACC GTT GCA GTA GTT CTC |
| | | CAG (SEQ ID NO: 19) |
| PPI1-Fc | pFUSE-hIgGl-Fc2 | 5′ ATG ATA TCA GGC CCT GTT GGT GCA CTT CCT |
| | | (SEQ ID NO: 20) |
| | | 3′ TAG ATC TAC CGC CGC CAC CGT TGC AGT AGT TCT |
| | | CCA (SEQ ID NO: 21) |
| PPII-Fc | pFastBacI | 5′ AAT TTC TAG AAT GGC CCT GT (SEQ ID NO: 22) |
| | | 3′ AAT TCT CGA GCT AGT TGC AGT AG (SEQ ID NO: 23) |
| PPI2 | pCR4-TOPO | 5′ ATG GCC CTG TGG ATG CGC TTC (SEQ ID NO: 24) |
| | | 3′ AGA TCT ACC GCC GCC ACC GTT GCA GTA GTT CTC |
| | | CAG (SEQ ID NO: 25) |
| PPI2-Fc | pFUSE-hIgG1-Fc2 | 5′ ATG ATA CAT GGC CCT GTG GAT GCG CTT |
| | | CCT (SEQ ID NO: 26) |
| | | 3′ TAG ATC TAC CGC CGC CAC CGT TGC AGT |
| | | AGT TCT CCA (SEQ ID NO: 27) |
| PPI2-Fc | pFastBacI | 5′ AAT TTC TAG AAT GGC CCT GT (SEQ ID NO: 28) |
| | | 3′ AAT TCT CGA GCT AGT TGC AGT AG (SEQ ID NO: 29) |

### Surface Plasmon Resonance

Kinetics constants of interactions between mouse or human FcRn and PPI 1-Fc, PPI2-Fc or IgG1 were determined using Biacore 2000 (GE Healthcare), as detailed in FIG. 9A-C.

### Mice, In Vivo Treatments and Diabetes Induction

G9C8 and NOD 8- to 15-wk-old primiparous pregnant mice, housed in specific pathogen-free conditions, were retro-orbitally injected with 100 gg PPI-Fc (an equimolar mixture of PPI1-Fc and PPI2-Fc), with equimolar amounts of IgG1 or PPI, or with PBS vehicle alone at embryonic day (E)16. After birth, 3.5-wk-old G9C8 newborns were immunized with 50 μg PPI_{B15-23} peptide and 100 μg CpG (5) and boosted 2 wk later. Diabetes was monitored by glycosuria and confirmed by hyperglycemia when positive. For FcRn and vascular cell adhesion molecule (VCAM)-1 blocking experiments, PPI-Fc treatment was performed 24 h after intravenous (i.v.) injection of 100 μg IgG (rituximab) or anti-VCAM-1 mAb (clone M/K2.7, produced in-house). For transfer experiments, 15×10⁶ splenocytes from the 14-wk-old offspring of treated NOD mice were adoptively transferred into 4-to-6-wk-old NOD.scid recipients and their pancreata recovered for insulitis scoring as described (6). The study was approved by the Comite d′Ethique pour l'Experimentation Animale (P2.RM.117.09, CEEA34.SC.158.12).

### In Vivo PPI-Fc Imaging and ELISA Quantification

PPI-Fc and PPI proteins were conjugated with ALEXA FLUOR® (AF)680 using SAIVI Rapid Antibody/Protein labeling kit (INVITROGEN™). G9C8 and β₂m^{-/-} primiparous pregnant mice (E18) were retro-orbitally injected with 100 μg PPI-Fc, equimolar amounts of PPI, or PBS vehicle. Fluorescence was detected using the Fluobeam imaging system (Fluoptics) at a 690 nm excitation and >700 nm emission wavelengths, with 50-100 ms exposures. After imaging, blood and urine were collected for ELISA quantification, with standard curves obtained by sequential dilutions of PPI-Fc and PPI proteins. Both PPI-Fc and PPI were captured with plate-coated H-86 anti-insulin Ab (Santa Cruz). PPI-Fc was detected with a horseradish peroxidase-labeled goat anti-human Fc Ab (Southern Biotech). PPI was revealed with an anti-proinsulin mAb (KL-1).

### In Vitro Proliferation and Cytotoxicity Assays

Bone-marrow-derived DCs (BMDCs) prepared from 6-wk-old G9C8 mice were pulsed for 8 h with 26 μM PPI_{B15-23}, PPI-Fc or PPI. Following maturation with 100 ng/ml lipopolysaccharide (LPS), they were co-cultured for 5 d with CFSE-labeled splenocytes from the 7-wk-old offspring of untreated G9C8 mice. Upon staining with PerCP-eFluor710-labeled anti-Vβ6, AF700-labeled anti-CD8a, APC-eFluor780-labeled anti-CD3ε mAbs (eBioscience), Brilliant Violet (BV)605-labeled anti-CD4 mAb (BioLegend) and Live/Dead Red (INVITROGEN™), cells were analyzed on a 16-color BD LSR Fortessa. Real-time cytotoxicity assays were performed with the xCELLigence system (ACEA Biosciences). Briefly, mouse fibroblast L cells were plated on 96-well E-plates, irradiated (5,000 rad) and rested for 2 h. FACS-sorted CD8⁻ T cells were added at 10:1 effector-target ratio in the presence of 10 nM PPI_{B15-23} peptide and impedance recorded every 5 min for 2 h, then every 15 min for an additional 3 h.

T-Cell Phenotyping and Quantitative Real-Time (qRT)-PCR

The following mAbs were used: PE-labeled anti-Foxp3, APC-eFluor780-labeled anti-CDR (eBioscience); APC-labeled anti-neuropilin-1 (NRP1; R&D); BV421-labeled anti-CD62L and anti-transforming growth factor (TGF)-β latency-associated peptide (LAP; clone TW7-16B4), BV570-labeled anti-CD44, BV605-labeled anti-CD4 and BV711-labeled anti-CD8a (BioLegend). Cells were additionally stained with Live/Dead Red and BV650-labeled K^{d} multimers loaded with PPI_{B15-23} (LYLVCGERG, SEQ ID NO: 30) or control TUM peptide (KYQAVTTTL, SEQ ID NO: 31), as described (18).

For qRT-PCR, G9C8 pregnant mice were retro-orbitally injected with 100 μg PPI-Fc or PBS vehicle at E16. After birth, G9C8 offspring were prime-boosted with 50 μg PPI_{B15-23} and 100 μg CpG at 3.5 and 5.5 wk. Blood was collected either before immunization or at d 0, 5, 19 and 30 after priming. Peripheral blood mononuclear cells were stained with APC-eFluor780-labeled anti-CD3ε (eBioscience), BV605-labeled anti-CD4 and BV711-labeled anti-CD8a (BioLegend) and sorted on a BD FACSAria III at 10 CD4⁺ or CD8⁺ cells/well into PCR plates. RNA was extracted by direct lysis for 2 min at 65° C. and multiple genes co-amplified as described (20) by semi-nested PCR with the primers listed in Table 4.

RNA was extracted from sorted CD8+ and CD4+ T-cells by direct lysis for 2 min at 65° C. Co-amplification of multiple genes was carried out as described (8,9). Briefly, RNA was reverse transcribed with murine leukemia virus reverse transcriptase (Applied Biosystems) for 60 min at 37° C. Semi-nested PCR was then performed with gene-specific primers (Eurogentec) and AmpliTaq Gold Polymerase (Applied Biosystems) by touch-down PCR. mRNA expression was normalized to Cd3ε.

TABLE 4
Oligonucleotide sequences of primers used for qRT-PCR

| Target | Primer | |
|---|---|---|
| gene | name | Primer Sequence |
| Cd3e | Cd3e-A | 5′ ACC AGT GTA GAG TTG ACG TG (SEQ ID NO: 32) |
| | Cd3e-B | 3′ TAT GGC TAC TGC TGT CAG GT (SEQ ID NO: 33) |
| | Cd3e- | 5′ GCT ACT ACG TCT GCT ACA CA (SEQ ID NO: 34) |
| Gzma | Gzma-A | 5′ TCA AAT ACC ATC TGT GCT GG (SEQ ID NO: 35) |
| | Gzma-B | 3′ AGA GGG AGC TGA CTT ATT GC (SEQ ID NO: 36) |
| | Gzma-C | 5′ GGG ATC TAC AAC TTG TAC GG (SEQ ID NO: 37) |
| Prf1 | Prf1-A | 5′ TCA CAC TGC CAG CGT AAT GT (SEQ ID NO: 38) |
| | Prf1-B | 3′ CTG TGG TAA GCA TGC TCT GT (SEQ ID NO: 39) |
| | Prf1-C | 5′ CAC AGT AGA GTG TCG CAT GT (SEQ ID NO: 40) |
| Fasl | Fasl-A | 5′ TTC ATG GTT CTG GTG GCT CT (SEQ ID NO: 41) |
| | Fasl-B | 3′ GAG CGG TTC CAT ATG TGT CT (SEQ ID NO: 42) |
| | Fasl-C | 5′ TGT ATC AGC TCT TCC ACC TG (SEQ ID NO: 43) |
| Tgfbr2 | Tglbr2-A | 5′ AGA TGC ATC CAT CCA CCT AA (SEQ ID NO: 44) |
| | Tgfbr2-B | 3′ TGC ACT CTT CCA TGT TAC AG (SEQ ID NO: 45) |
| | Tgfbr2-C | 5′ CGA TGT GAG ACT GTC CAC TT (SEQ ID NO: 46) |
| Tgfb1 | Tgfb1-A | 5′ ACC ATC CAT GAC ATG AAC CG (SEQ ID NO: 47) |
| | Tgfb1-B | 3′ CAA TCA TGT TGG ACA ACT GC (SEQ ID NO: 48) |
| | Tgfb1-C | 5′ GCT ACC ATG CCA ACT TCT GT (SEQ ID NO: 49) |

### DC Migration and PPI-Fc Cellular Uptake

AF647-conjugated PPI-Fc (100 μg) was i.v. injected into pregnant G9C8 mice at E19. Newborns were sacrificed 24 h later and thymi, spleens and blood from 2-4 mice pooled together. For thymi, single-cell suspensions were obtained by enzymatic digestion (10). PPI-Fc⁺ events were identified by gating on live cells of each subset. To evaluate the migratory capacity of different DC subsets to the thymus, hemolysed total blood cells from 1-day-old G9C8 newborns were transferred into 5-wk-old NOD.scid mice. After 24 h, mice were sacrificed and isolated thymic cells stained for enumeration of DC subsets.

### Statistics

Data from separate experiments is depicted as mean±SEM. Statistical significance (p<0.05) was assigned with the two-tailed tests detailed in each figure legend using GraphPad Prism 5.

### Results

PPI-Fc Binds to FcRn with High Affinity and is Transferred Through the Placenta

Unlike humans, mice harbor two Ins genes: Ins1 is predominantly expressed in the pancreas, while Ins2 is expressed in the thymus. Ins1 and Ins2 were therefore fused with the N-terminus of the CH2-CH3 Fc domain from human IgG1 to obtain PPI1-Fc and PPI2-Fc fusion proteins (FIG. 9A-B). Since FcRn is crucial for Fey-dependent transcytosis across the placenta, we evaluated the binding affinities of PPI1-Fc and PPI2-Fc on immobilized murine and human FcRn using surface plasmon resonance (FIG. 9C). Both proteins displayed efficient binding (Table 5), with a slightly higher affinity for mouse (K_{D}≈6 nM) than for human FcRn (K_{D}≈15 nM).

TABLE 5
Affinity measurements of PPI-Fc binding to FcRn by surface plasmon
resonance. Values of the kinetic rate constants (kₐ and k_{d}) and equilibrium
dissociation constant (K_{D}) obtained by global analyses of sensorgrams
obtained after injection of the indicated proteins (0.78-200 nM)
on sensor chips coated with mouse or human FcRn. The kinetic model
for Langmuir binding with drifting baseline was used for fitting
of the binding curves.

| | | kₐ (×10⁵ M⁻¹ s⁻¹) | k_{d} (×10⁻³ s⁻¹) | K_{D} | |
|---|---|---|---|---|---|
| FcRn | Analyte | (mean ± SEM) | (mean ± SEM) | (nM) | Chi² |
| Mouse | PPI1-Fc | 1.67 ± 0.01 | 1.04 ± 0.02 | 6.2 | 0.8 |
| | PPI2-Fc | 2.92 ± 0.01 | 1.59 ± 0.03 | 5.4 | 2.0 |
| | hIgG1 | 1.63 ± 0.06 | 2.48 ± 0.03 | 1.5 | 14.0 |
| human | PPI1-Fc | 2.07 ± 0.01 | 3.11 ± 0.06 | 15.0 | 14.2 |
| | PPI2-Fc | 2.08 ± 0.02 | 3.18 ± 0.07 | 15.3 | 26.4 |
| | hIgG1 | 7.65 ± 0.05 | 4.92 ± 0.04 | 6.4 | 8.3 |

For in vivo studies, we employed the G9Cα^{-/-}.NOD (G9C8) mouse (5), which expresses a transgenic T-cell receptor (TCR) derived from the diabetogenic G9C8 CD8⁺ T-cell clone (11) recognizing the H-2K^{d}-restricted PPI_{B15-23} epitope. These mice develop diabetes rapidly (4-8 d) after PPI_{B15-23} peptide immunization with CpG adjuvant (12). Since the PPI_{B15-23} epitope is shared between PPI1-Fc and PPI2-Fc, a 1:1 mix of both proteins (hereafter designated PPI-Fc) was used for subsequent experiments. To assess the efficiency of placental transfer, 100 μg of AF680-labeled PPI-Fc were i.v. injected into pregnant G9C8 mice at E18. In vivo imaging demonstrated selective PPI-Fc accumulation in the uterine horns (FIG. 10A) and fetuses (FIG. 10B) 24 h after injection. This transfer was Fc-dependent, as injection of Fc-devoid PPI into pregnant G9C8 mice led to its rapid (within 1 min) renal accumulation, without detectable placental transfer (FIG. 10A-B). Furthermore, interaction with the FcRn was also required, since PPI-Fc administration to β₂m^{-/-} mice devoid of functional FcRn expression (13) did not result in any detectable transfer (FIG. 10A-B), as previously observed with FcRn^{-/-} mice (14). Interestingly however, PPI-Fc was detectable at the vascularized placental interface (FIG. 10B), suggesting that fusion to the Fc domain stabilizes PPI and increases its half-life. Indeed, PPI-Fc fluorescence was still detectable in 7-day-old newborn G9C8 mice, i.e., 9 d after administration to their pregnant mothers at E18 (FIG. 10C).

Quantitative ELISA measurements of intact PPI-Fc were subsequently performed. While serum PPI-Fc concentrations became barely detectable within 24 h after injection in G9C8 pregnant mice (FIG. 10D), they remained stable for 48 h in their fetuses, reaching concentrations of ˜0.75 ng/μl and documenting PPI-Fc integrity after transfer. No serum PPI-Fc accumulation was observed in either β₂m^{-/-} pregnant mice or their fetuses. Analyses of urine PPI-Fc from pregnant females gave symmetrical results (FIG. 10E): G9C8 mice excreted limited amounts, mostly during the first hours after injection, while β₂m^{-/-} mice continued to excrete PPI-Fc even 24 h post-injection. Similarly, an ELISA for PPI detected rapid and steady PPI urinary excretion in PPI- but not PPI-Fc-treated G9C8 pregnant mice (FIG. 10F).

Taken together, these data indicate that efficient PPI-Fc transplacental transfer is dependent on Fc-FcRn binding. Since TCR expression is first detected in the thymus at E17 and given that maternally administered PPI-Fc persisted in the fetal circulation for at least 48 h and remained detectable in newborn mice, PPI-Fc was injected into pregnant G9C8 mice with a single 100 μg dose at E16 for subsequent experiments.

Transplacentally Delivered PPI-Fc Primes G9C8 TCR-Transgenic T-Cells and Protects from Diabetes

G9C8 mice harbor increased proportions of splenic CD8⁺ T-cells and reduced CD4⁺ T-cells compared to non-transgenic NOD mice (not shown). As in NOD mice, ˜15% of CD4⁺ T-cells are Foxp3⁺ Tregs, but with higher NRP1⁺ thymic-derived Treg fractions (˜90% of total Tregs vs. ˜70% in NOD mice). Both CD4⁺ and CD8 T-cells express the transgenic Vβ6 chain, but only CD8⁺ T-cells stain with PPI_{B15-23}-loaded K^{d} multimers. In vitro CFSE proliferation assays showed that G9C8 CD8⁺ T-cells are stimulated by PPI-Fc but not by Fc-devoid PPI (FIG. 11A), hence demonstrating efficient PPI_{B15-23} cross-presentation. CD4⁺ T-cells proliferated upon stimulation with both PPI-Fc and PPI (FIG. 11B).

Since PPI-Fc is transferred through the placenta and cross-primes G9C8 TCR-transgenic T-cells in vitro, pregnant G9C8 mice were treated with 100 μg PPI-Fc at E16. Following delivery, their offspring were immunized with PPI_{B15-23} peptide and CpG at 3.5 and 5.5 wk of age to induce diabetes and prospectively followed. As controls, equimolar amounts of recombinant IgG1 (i.e. irrelevant protein with preserved FcRn binding), PPI (i.e. cognate Ag with no FcRn binding) or PBS vehicle were injected. Diabetes development was rapid and synchronous in the offspring of control-treated mice, mostly within 1 wk after prime immunization (FIG. 11C). In contrast, the offspring of PPI-Fc-treated mice were significantly protected, showing reduced and delayed diabetes incidence (70% diabetes-free mice vs. 22-27% at the end of the 30-d follow-up; p<0.0001). Since no difference was observed for IgG1, PPI and PBS groups, PBS vehicle alone was used as control for subsequent experiments.

We next asked whether PPI-Fc priming of G9C8 TCR-transgenic T-cells also occurred in vivo following transplacental transfer and diabetes induction by PPI_{B15-23} prime-boost immunization. Indeed, increased frequencies of splenic CD8⁺ T-cells were observed in the 7-wk-old offspring of PPI-Fc-treated mice (FIG. 11D; 8.1±0.5% vs. 6.4±0.3% in control-treated animals; p=0.01), which was limited to the memory (CD44⁺) subset (FIG. 11E; 10.4±2.1% vs. 5.1±0.8%; p=0.02), while naïve (CD62L⁺CD44⁺) fractions were similar irrespective of treatment. The limited size of this memory CD8⁺ fraction (5-10% of total CD8⁺ T-cells) suggests that PPI_{B15-23} prime-boost immunization is relatively inefficient at recruiting G9C8 TCR-transgenic CD8⁺ T-cells, probably because of their low avidity, and that prior PPI-Fc maternal treatment enhances such recruitment.

### The Offspring of PPI-Fc-Treated G9C8 Mice Harbors CD8⁺ T-Cells Displaying Impaired Cytotoxicity and Increased Numbers of Thymic-Derived Tregs Expressing TGF-β

The increased frequency of CD8⁺ T-cells in the offspring of PPI-Fc-treated mice was opposite to what expected, in light of the protective effect of PPI-Fc on diabetes development. We therefore analyzed the phenotype of circulating CD8⁺ T-cells in the progeny of PPI-Fc- and PBS-treated mice at different time points before and after PPI_{B15-23} immunization by qRT-PCR (FIG. 12A). While undetectable before PPI_{B15-23} immunization, the expression of granzyme A (Gzma), perforin (Prf1), and Fas ligand (Fasl) was increased after immunization, and more so in PBS-treated than in PPI-Fc-treated mice. Conversely, TGF-β receptor 2 (Tgfbr2) expression was increased in the PPI-Fc-treated group, but not in the PBS-treated group. In vitro cytotoxicity assays under limiting (10 nM) PPI_{B15-23} peptide concentrations confirmed that CD8⁺ T-cells from PPI-Fc-treated mice were less cytotoxic (FIG. 12B). Taken together, these results show that prior maternal PPI-Fc treatment imprints the phenotype of later CD8⁺ T-cell responses, making them less cytotoxic and more prone to TGF-β-mediated regulation.

To identify potential sources of TGF-β, we analyzed splenic CD4⁺ T-cells. Total CD4⁺ T-cell numbers were not significantly different between treatment groups (FIG. 11D). However, Foxp3⁺ Tregs were more abundant in the offspring of PPI-Fc-treated mothers (FIG. 12C; 25.7±3.6% vs. 17.0±2.5% in control-treated animals; p=0.05), without significant differences in Foxp3⁺CD4⁺ T-cells. This Treg increase was exclusively made up by thymic-derived (NRP-1⁺) Foxp3⁺ Tregs (FIG. 12D; 18.8±3.3% vs. 13.7±3.5%; p=0.0003), while the percentage of peripheral (NRP-1⁻) Tregs was similar in both treatment groups (5.3±3.9% vs. 4.4±2.8%), as was expression of surface TGF-β LAP in FoxP3⁺CD4⁺ Tregs following in vitro activation (FIG. 12E and data not shown). Finally, qRT-PCR analysis on circulating CD4⁺ T-cells showed a higher TGF-β (Tgfb1) expression in the progeny of PPI-Fc-treated mice (FIG. 12F; 0.21±0.09 vs. 0.05±0.00; p=0.03) which, in light of the Treg-specific TGF-β LAP expression (FIG. 12E), can be attributed to the increased Treg numbers observed in PPI-Fc-treated mice. Taken together, these data show that maternal PPI-Fc treatment protects G9C8 newborns from diabetes development and that such protection is associated with increased priming of CD8⁺ T-cells that are less cytotoxic; and with an enrichment in thymic-derived Tregs expressing TGF-β.

### Diabetes Protection is Dependent on Ferrying of PPI-Fc to the Thymus by Migratory DCs

Given the observed effect of PPI-Fc on thymic-derived Tregs, we investigated whether fluorescence-labeled PPI-Fc was capable of reaching the thymus. Twenty-four hours after injection into pregnant mice at E18, PPI-Fc was readily detected in fetal thymi, whereas PPI-treated mice showed no signal (FIG. 13A). No fluorescence was detected in the spleen. In line with the in vivo imaging data, PPI-Fc was still weakly detectable in the thymi of 5-d newborn mice, i.e. 7 d after PPI-Fc maternal treatment (FIG. 13A).

Next, we asked whether Ag-presenting cells were responsible for ferrying PPI-Fc to the thymus. A population of migratory CD8^{lo}CD11b⁺SIRPα⁺ conventional (c)DCs is known to transport blood-borne Ags to the thymus and promote central tolerance via negative selection of Ag-specific Teffs and Treg positive selection. CD11c^{int}B220⁺PDCA-1⁺ plasmacytoid (p)DCs have also been suggested to ferry peripherally acquired Ags and participate in central tolerance. Hence, we first determined the DC subsets capable of migrating to the thymus. Total blood cells from neonatal G9C8 mice were injected into 6-wk-old NOD.scid mice. Thymi were removed 24 h later and DC subsets analyzed. Migratory SIRPα⁺ cDCs were significantly enriched in the thymi of adoptively transferred mice (FIG. 13B-C; 0.67±0.54% vs. 0.02±0.02% in control mice; p=0.05), while thymic resident (CD8^{hi}CD11b⁻Sirpα⁻) cDCs and pDCs were not.

To verify whether migratory cDCs were capable of uptaking and ferrying PPI-Fc to the thymus, fluorescently labeled PPI-Fc was injected into pregnant G9C8 mice 24 h before delivery (E19). Thymi were then removed from their newborns and analyzed for PPI-Fc fluorescence in different thymic subsets (FIG. 13D). Only SIRPα⁺ cDCs carried PPI-Fc in ˜11% of cells, while neither other DC subsets nor medullary thymic epithelial cells (mTECs; CD45⁻EpCAM⁺CDR1⁻) displayed any detectable fluorescence. Moreover, SIRPα⁺ cDCs were loaded with PPI-Fc not only in the thymus, but also, to a lesser extent, in peripheral blood (13.1% vs. 6.8%; FIG. 13E), suggesting that PPI-Fc is uptaken in the periphery and subsequently ferried to the thymus. When analyzing other thymic, blood and spleen subsets (FIG. 14), SIRPα⁻ cDCs, B-cells and macrophages were also loaded with PPI-Fc in peripheral blood (6.7%, 3.5% and 1.9%, respectively), but only B-cells displayed some fluorescence in the thymus (2.5%). In line with the results of ex vivo whole-organ imaging (FIG. 13A), PPI-Fc uptake was negligible in the spleen.

We then asked whether FcRn-mediated PPI-Fc transplacental transfer and SIRPα⁺ cDC migration were responsible for the protective effect of PPI-Fc on diabetes development.

Pregnant mice were i.v. injected 24 h prior to PPI-Fc treatment with either an IgG isotype control, in order to compete with PPI-Fc for FcRn binding, or with an anti-VCAM-1 mAb, since SIRPα⁺ cDC migration is dependent on VLA-4-VCAM-1 interactions. As before, diabetes was then induced in the offspring by prime-boost PPI_{B15-23} immunization. While PBS pre-treatment did not reduce the PPI-Fc protective effect in the offspring (FIG. 13F), the isotype control IgG partially inhibited this protection (49% vs. 71% diabetes-free mice; p=0.04). More strikingly, anti-VCAM-1 mAb pre-treatment completely abolished the PPI-Fc diabetes protection, with only 18% of mice remaining diabetes-free (p<0.0001 and p=0.01 compared to PBS and isotype mAb pre-treatment, respectively). Although VCAM-1 is also essential for lymphocyte homing to inflamed tissues, including islets, the early single-dose treatment employed is unlikely to retain a blocking effect on islet infiltration, since it was administered 4 wk before diabetes induction.

Taken together, these results show that PPI-Fc is ferried to the thymus by migratory SIRPα⁺ cDCs, and that both transplacental delivery through FcRn and cDC migration are needed for PPI-Fc-mediated diabetes protection.

### The Offspring of PPI-Fc-Treated NOD Mice Displays Milder Insulitis and Less Diabetogenic Splenocytes

Finally, we evaluated whether PPI-Fc could prevent diabetes in polyclonal NOD mice. We i.v. injected 200 μg of PPI-Fc or PBS into pregnant NOD mice at E16. The pre-diabetic female progeny of these mice was sacrificed at 14 wk, and their splenocytes adoptively transferred into 4- to 6-wk-old NOD.scid mice. Pancreata from donor NOD mice recovered for insulitis scoring displayed milder islet infiltration in females born from mothers treated with PPI-Fc compared to controls (FIG. 15A; p=0.007). This was paralleled by a significantly lower diabetogenic potency of splenocytes from the offspring of PPI-Fc-treated NOD mice (FIG. 15B). While, in line with previous reports, 60% of NOD.scid recipients receiving splenl ocytes from control NOD donors developed diabetes, only 37% of those adoptively transferred from PPI-Fc-treated animals became diabetic (p=0.04). Taken together, these data show that PPI-Fc transplacental delivery blunts the insulitis and the splenocyte diabetogenic activity of polyclonal NOD mice.

### Discussion

The tolerogenic Ag vaccination strategies explored to date for T1D have targeted peripheral tolerance mechanisms. Here, we undertook a different strategy to target the earliest checkpoint in autoimmune progression, namely the development of central tolerance in the thymus. Previous reports suggest that it is possible to ‘upgrade’ central tolerance by administering Ags either intra-thymically or in the periphery. In the latter case, a key role is played by migratory DCs that ferry these Ags to the thymus, with a direct thymic entry of soluble Ags also documented. We aimed at translating this concept into a therapeutically viable strategy.

Several lines of evidence show that defective central tolerance is involved in T1D development. First, Ins2^{-/-} NOD mice develop accelerated diabetes (15) due to absent thymic PPI expression (16). Second, the human INS variable number of tandem repeats (VNTR) polymorphic region, which ranks as the second most powerful T1D susceptibility locus after DQB1, modulates INS expression in the thymus (17). However, this knowledge has not translated into therapeutic strategies aimed at boosting central tolerance ab initio. The notion that autoimmune activation against PPI appears already during the first 9-18 mo of life, as witnessed by anti-insulin auto-Abs, lends further rationale to these strategies.

Transferred through the placental FcRn pathway, which physiologically delivers maternal IgGs, PPI-Fc fusion proteins were efficiently delivered to fetuses upon administration to pregnant G9C8 mice. The mechanism was Fc-FcRn-dependent, since delivery did not occur in the absence of either and diabetes protection was inhibited with excess IgG. Subsequent ferrying of PPI-Fc to the thymus by migratory SIRPα⁺ cDCs was also essential, since diabetes protection was lost when cDC migration was inhibited. Surprisingly, transplacental PPI-Fc delivery resulted in enhanced rather than decreased recruitment of CD8⁺ Teffs in the periphery upon PPI_{B15-23} immunization. However, these CD8⁺ Teffs were less cytotoxic. The low affinity G9C8 TCR and reduced PPI_{B15-23} availability due to the requirement for PPI-Fc cross-presentation may favor CD8⁺ Teff expansion and limit the effect on thymic negative selection. Nonetheless, this low TCR affinity was sufficient to promote thymic positive selection of TGF-β1-expressing CD4⁺ Tregs, possibly regulating more efficiently CD8⁺ Teffs, which expressed higher TGF-βR2 levels. This latter finding is reminiscent of data in both NOD mice and T1D patients, showing that Teff susceptibility to Treg suppression is a key parameter for immune tolerance and is reduced in T1D.

In summary, the therapeutic mechanism was dependent on FeRn-mediated PPI-Fc transfer and cDC migration to the thymus, and resulted in impaired Teff cytotoxicity and enhanced selection of thymic Tregs. Moreover, we recently applied a similar strategy of transplacental Ag-Fc administration in the CD4⁺ hemagglutinin (HA)₁₁₀₋₁₁₉ TCR-transgenic 6.5 mouse model to fully dissect therapeutic mechanisms (Example 1). HA-Fc ferrying to the thymus was also observed in this model and three differences were highlighted. First, HA-Fc was uptaken by SIRPα⁺ cDCs and, to a lesser extent, by macrophages. This may be due to the higher molecular weight of HA-Fc (65 vs. 38 kDa for PPI-Fc) and by HA interaction with different cell types through sialic acid moieties expressed on cell membranes, independent of Fc. Second, marginally reduced rather than increased CD4⁺ Teffs were observed in the periphery (but not in the thymus). This discrepancy may be due to peripheral effects mediated by HA-Fc-loaded macrophages and to the higher affinity of the HA₁₁₀₋₁₁₉ TCR, which may favor activation-induced apoptosis upon high-dose Ag encounter. Third, both thymic-derived and peripheral Ag-specific Tregs were induced.

The diabetes protection afforded by transplacental PPI-Fc delivery is noteworthy when considering the challenges posed by the G9C8 model, namely disease aggressiveness harnessed through PPI_{B15-23} immunization and the need for PPI-Fc cross-presentation to exert effects on CD8⁺ Teffs. Moreover, a single 100 μg PPI-Fc dose was sufficient to confer protection. This was likely favored by the Fc moiety conferring enhanced stability, since PPI-Fc remained detectable in the offspring as long as 9 d after maternal treatment. Another key issue was whether inducing tolerance to PPI alone would be sufficient to impact a polyclonal autoimmune T-cell repertoire. Adoptive transfer of NOD splenocytes suggest that this is the case. Follow-up studies are needed to explore whether such protection is maintained in NOD mice prospectively observed for diabetes development. Of further note, applications could reach beyond autoimmunity, as this strategy was also effective at promoting neo-Ag-specific tolerance towards clotting factor VIII (FVIII) in FVIII^{-/-} mice challenged with therapeutic FVIII (Example 1).

Employing a single PPI-Fc dose to induce long-lasting tolerance is attractive for translation to genetically at-risk children. From this perspective, a puzzling observation is that the risk conferred by T1D mothers is half than that transmitted by T1D fathers (3-4% vs. 6-8% at 20 yr). This relative protection seems linked to transplacental transfer of maternal auto-Abs. One mechanism for this protection may be the transfer of Ab-bound islet Ags through placental FcRn, similar to what observed with PPI-Fc.

A combination of several Ag-Fc therapeutics could be used to induce broad immune tolerance, and islet Ags displaying defective thymic expression may be particularly suitable to this end. Given its initiating role, PPI remains an Ag of choice and enrollment of newborns based on expression of T1D-susceptible INS VNTR alleles may be considered.

### REFERENCES FOR EXAMPLE 2

1. Achenbach P, Barker J, Bonifacio E: Modulating the natural history of type 1 diabetes in children at high genetic risk by mucosal insulin immunization. Curr Diab Rep 2008; 8:87-93
2. Billingham R E, Brent L, Medawar PB: Actively acquired tolerance of foreign cells. Nature 1953; 172:603-606
3. Mold J E, Michaelsson J, Burt T D, Muench M O, Beckerman K P, Busch M P, Lee T H, Nixon D F, McCune J M: Maternal alloantigens promote the development of tolerogenic fetal regulatory T cells in utero. Science 2008;322:1562-1565
4. Guerau-de-Arellano M, Martinic M, Benoist C, Mathis D: Neonatal tolerance revisited: a perinatal window for Aire control of autoimmunity. J Exp Med 2009; 206:1245-1252
5. Wong F S, Siew L K, Scott G, Thomas I J, Chapman S, Viret C, Wen L: Activation of insulin-reactive CD8 T cells for development of autoimmune diabetes. Diabetes 2009; 58:1156-1164
6. Brezar V, Culina S, Gagnerault M C, Mallone R: Short-term subcutaneous insulin treatment delays but does not prevent diabetes in NOD mice. Eur J Immunol 2012; 42:1553-1561
7. Garboczi D N, Utz U, Ghosh P, Seth A, Kim J, VanTienhoven E A, Biddison W E, Wiley D C: Assembly, specific binding, and crystallization of a human TCR-alphabeta with an antigenic Tax peptide from human T lymphotropic virus type 1 and the class I MHC molecule HLA-A2. J Immunol 1996; 157:5403-5410
8. Luce S, Lemonnier F, Briand J P, Coste J, Lahlou N, Muller S, Larger E, Rocha B, Mallone R, Boitard C: Single insulin-specific CD8+ T cells show characteristic gene expression profiles in human type 1 diabetes. Diabetes 2011; 60:3289-3299
9. Scotto M, Afonso G, Osterbye T, Larger E, Luce S, Raverdy C, Novelli G, Bruno G, Gonfroy-Leymarie C, Launay O, Lemonnier F A, Buus S, Carel J C, Boitard C, Mallone R: HLA-B7-restricted islet epitopes are differentially recognized in type 1 diabetic children and adults and form weak peptide-HLA complexes. Diabetes 2012; 61:2546-2555
10. Derbinski J, Schulte A, Kyewski B, Klein L: Promiscuous gene expression in medullary thymic epithelial cells mirrors the peripheral self. Nat Immunol 2001; 2:1032-1039
11. Wong F S, Karttunen J, Dumont C, Wen L, Visintin I, Pilip I M, Shastri N, Pamer E G, Janeway C A, Jr.: Identification of an MHC class I-restricted autoantigen in type 1 diabetes by screening an organ-specific cDNA library. Nat Med 1999; 5:1026-1031
12. Wong F S, Siew L K, Scott G, Thomas I J, Chapman S, Viret C, Wen L: Activation of insulin-reactive CD8 T cells for development of autoimmune diabetes. Diabetes 2009; 58:1156-1164
13. Roopenian D C, Akilesh S: FcRn: the neonatal Fc receptor comes of age. Nat Rev Immunol 2007; 7:715-725
14. Gupta N, Culina S, Meslier Y, Dimitrov J, Arnoult C, Delignat S, Gangadharan B, Lecerf M, Justesen S, Gouilleux-Gruart V, Salomon B L, Scott D W, Kaveri S V, Mallone R, Lacroix-Desmazes S: Regulation of immune responses to antigens and protein therapeutics by transplacental induction of central and peripheral T-cell tolerance. Sci Transl Med 2015; 7:275ra221
15. Thebault-Baumont K, Dubois-Laforgue D, Krief P, Briand J P, Halbout P, Vallon-Geoffroy K, Morin J, Laloux V, Lehuen A, Carel J C, Jami J, Muller S, Boitard C: Acceleration of type 1 diabetes mellitus in proinsulin 2-deficient NOD mice. J Clin Invest 2003; 111:851-857
16. Faideau B, Lotton C, Lucas B, Tardivel I, Elliott J F, Boitard C, Carel J C: Tolerance to proinsulin-2 is due to radioresistant thymic cells. J Immunol 2006; 177:53-60
17. Pugliese A, Zeller M, Fernandez A, Jr., Zalcberg L J, Bartlett R J, Ricordi C, Pietropaolo M, Eisenbarth G S, Bennett S T, Patel D D: The insulin gene is transcribed in the human thymus and transcription levels correlated with allelic variation at the INS VNTR-IDDM2 susceptibility locus for type 1 diabetes. Nat Genet 1997; 15:293-297
18. Leisner C, Loeth N, Lamberth K, Justesen S, Sylvester-Hvid C, Schmidt E G, Claesson M, Buus S, Stryhn A: One-pot, mix-and-read peptide-MHC tetramers. PLoS One 2008; 3:e1678
19. Gangadharan B, Delignat S, Ollivier V, Gupta N, Madman N, Kaveri S V, Lacroix-Desmazes S. Role of coagulation-associated processes on factor VIII immunogenicity in a mouse model of severe hemophilia A. J Thromb Haemost. 2014 December; 12(12):2065-9.
20. Luce S, Lemonnier F, Briand J P, Coste J, Lahlou N, Muller S, Larger E, Rocha B, Mallone R, Boitard C: Single insulin-specific CD8+ T cells show characteristic gene expression profiles in human type 1 diabetes. Diabetes 2011; 60:3289-3299

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A recombinant polypeptide construct comprising
a targeting moiety that binds neonatal Fc receptor (FcRn); and
an antigenic moiety comprising at least one antigen or antigenic determinant, wherein said antigenic moiety does not comprise a full-length FVIII with tyrosine at position 1680 or a segment of FVIII with tyrosine at position 1680.

2. The recombinant polypeptide construct of claim 1, wherein said at least one antigen is preproinsulin (PPI) or other pancreatic beta-cell antigen or an antigenic fragment thereof.

3. The recombinant polypeptide construct of claim 1, wherein said at least one antigen is FVIII that does not bind to, or exhibits reduced binding to, von Willebrand Factor (vWF).

4. The recombinant polypeptide construct of claim 3, wherein position 1680 of said FVIII is not tyrosine.

5. The recombinant polypeptide construct of claim 1, wherein said targeting moiety is selected from the group consisting of: human Fc γ ¼; a portion of human Fc γ ¼ sufficient to permit binding of said recombinant polypeptide construct to said FcRn receptor; monomeric Fc γ; and a Fc heterodimer.

6. The recombinant polypeptide construct of claim 1, wherein said segment of FVIII is a B domain deleted factor VIII (BDD FVIII).

7. A method of eliciting immune tolerance to at least one antigen or antigenic determinant of interest in a subject in need thereof comprising
administering to said subject a recombinant polypeptide construct comprising
a targeting moiety that binds neonatal Fc receptor (FcRn); and
an antigenic moiety comprising at least one antigen or antigenic determinant wherein said antigenic moiety does not comprise a full-length FVIII with tyrosine at position 1680 or a segment of FVIII with tyrosine at position 1680.

8. The method of claim 7, wherein the subject is a fetus and administration is performed in utero.

9. The method of claim 7, wherein the step of administering is performed transplacentally by administering the recombinant polypeptide construct to the mother.

10. The method of claim 7, wherein said at least one antigen is preproinsulin (PPI) or other pancreatic beta-cell antigen or an antigenic fragment thereof

11. The method of claim 7, wherein said at least one antigen is FVIII that does not bind to, or exhibits reduced binding to, von Willebrand Factor (vWF).

12. The method of claim 11, wherein position 1680 of said FVIII is not tyrosine.

13. The method of claim 7, said wherein targeting moiety is selected from the group consisting of: human Fc γ¼; a portion of human Fc γ ¼ sufficient to permit binding of said recombinant polypeptide construct to said FcRn receptor; monomeric Fc γ; and a Fc heterodimer.

14. The method of claim 7, wherein said segment of FVIII is a B domain deleted factor VIII (BDD FVIII).

15. A method of eliciting immune tolerance to an antigen of interest in an offspring of a female, comprising
during gestation of said offspring by said female, administering to said female a recombinant polypeptide construct comprising
a targeting moiety that binds neonatal Fc receptor (FcRn); and
an antigenic moiety comprising at least one antigen or antigenic determinant wherein said antigenic moiety does not comprise a full-length FVIII with tyrosine at position 1680 or a segment of FVIII with tyrosine at position 1680.

16. A method of inducing an increase of thymic and/or peripherally derived regulatory T cells (Tregs) and/or a decrease in conventional T cells specific for an antigen of interest in a fetus, comprising
delivering to said fetus a recombinant polypeptide construct comprising
a targeting moiety that binds neonatal Fc receptor (FcRn); and
an antigenic moiety comprising at least one antigen or antigenic determinant wherein said antigenic moiety does not comprise a full-length FVIII with tyrosine at position 1680 or a segment of FVIII with tyrosine at position 1680.
